(19) United States
(12) Patent Application Publication
(10) Pub. No.: US 2004/0235830 A1
(43) Pub. Date: Nov. 25, 2004

(54) Substituted cyclohexane derivatives and the use thereof in medicaments for treating cardiovascular diseases

(76) Inventors: Susanne Rohrig, Essen (DE); Andreas Stolle, Wuppertal (DE); Julio C. Castro-Palomino, Premia de Mar (DE); Helmut Haning, Wuppertal (DE); Gabriele Handke-Erguden, Wulfrath (DE); Noemi Daviu-Folguera, Barcelona (ES); Holger Paulsen, Wuppertal (DE); Josef Pernerstorfer, Wuppertal (DE); Stephan-Nicholas Wirtz, Wuppertal (DE); Henning Steinhagen, Sulzbach (DE); Wolfgang Thielemann, Wuppertal (DE); Erwin Bischoff, Wuppertal (DE); Ulrich Ebbinghaus-Kintscher, Dortmund (DE); Peter Ellinghaus, Wuppertal (DE); Joachim Hutter, Wuppertal (DE); Thomas Krahn, Hagen (DE); Frank Wunder, Wuppertal (DE); Klemens Lustig, Wuppertal (DE); Joachim Schuhmacher, Wuppertal (DE); Frank Sussmeier, Wuppertal (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/432,573

(22) PCT Filed: Nov. 12, 2001
(86) PCT No.: PCT/EP01/13062

(30) Foreign Application Priority Data
Nov. 24, 2000 (DE) 100 58 461.6

(51) Int. Cl. A61K 31/54; A61K 31/537; A61K 31/496; A61K 31/445; A61K 31/426; A61K 31/421

(52) U.S. Cl. 514/227.5; 514/237.5; 514/255.03; 514/317; 514/365; 514/374; 514/396; 514/424

(57) ABSTRACT

The invention relates to substituted cyclohexane derivates of formula (I), a method for the production thereof and the use thereof in medicaments, particularly for preventing and/or treating cardiovascular diseases, diseases of the urogenital tract and cerebrovascular diseases.

The present invention relates to substituted cyclohexane derivatives, a process for the preparation thereof and the use thereof in medicaments, in particular for preventing and/or treating cardiovascular diseases, diseases of the urogenital tract and cerebrovascular diseases.

Coronary heart diseases still represent the commonest cause of death in Western industrialized nations. Although numerous medicaments such as organic nitrates, beta blockers, calcium channel blockers and potassium channel openers are employed for treatment, the efficacy of these therapies is low. Thus, only a slight improvement in the endurance of the heart can be achieved and, moreover, disappears again after discontinuation of the medicaments.

The probability that potassium channels are open in the cell membrane determines the level of the resting membrane potential. As the probability that potassium channels in the cell membrane are open increases, the resting membrane potential is shifted in the direction of the potassium equilibrium potential, and thus the membrane becomes hyperpolarized. As a consequence thereof, the calcium influx through voltage-dependent calcium channels falls (functional calcium antagonism). This effect is particularly pronounced in the smooth muscles of arterial blood vessels, where the reduction in the intracellular calcium associated with the hyperpolarization leads to vasorelaxation.

The voltage-dependent and calcium-activated potassium channel of high conductance (synonyms: BigK, BK, MaxiK, slowpoke) which is expressed in the small resistance vessels is predominantly closed under resting conditions. However, if a high action potential frequency leads to a marked reduction in the membrane potential and/or to a large increase in the intracellular calcium concentration, the channel opens and the huge potassium efflux from the (muscle) cell effects a compensatory closing of the voltage-dependent calcium channels. A selective BigK modulator can therefore be used both for the treatment of angina pectoris and of essential hypertension (see Brenner et al., Nature 407, 2000, 870-876).

It is an object of the present invention now to provide novel substances for the prevention and/or treatment of cardiovascular diseases, diseases of the urogenital tract and cerebrovascular diseases.

The present invention relates to compounds of the formula (I)

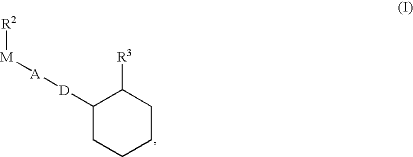

in which

M is a group —N(—R¹)— or an oxygen atom —O—,

A is a group —C(═O)— or —CH₂— or a chemical bond,

D is 5- or 6-membered heteroarylene with up to three heteroatoms from the series N, O and/or S or phenylene, each of which may be substituted up to three times, independently of one another, by halogen, hydroxyl, cyano, carboxyl, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

R¹ is hydrogen, benzyl, (C₂-C₆)-alkenyl, (C₁-C₆)-alkyl, optionally benzo-fused (C₃-C₈)-cycloalkyl,

where alkyl and cycloalkyl in turn may be substituted up to three times, independently of one another, by hydroxyl, amino, (C₁-C₆)-alkoxy, phenyl, 5- or 6-membered heterocyclyl with up to three heteroatoms from the series N, O and/or S, (C₃-C₈)-cycloalkyl or mono- or di-(C₁-C₆)-alkylamino,

(C₆-C₁₀)-aryl, 5- to 10-membered heteroaryl with up to three heteroatoms from the series N, O and/or S or 5- or 6-membered heterocyclyl with up to three heteroatoms from the series N, O and/or S,

where aryl, heteroaryl and heterocyclyl in turn may be substituted up to three times, independently of one another, by halogen, hydroxyl, oxo, cyano, nitro, trifluoromethyl, trifluoromethoxy, (C₁-C₆)-alkyl, (C₁-C₆)-alkoxy, (C₁-C₆)-alkoxycarbonyl, N-acetyl,N-methylamino or mono- or di-(C₁-C₆)-alkylamino,

R² is hydrogen, (C₁-C₆)-alkyl, (C₃-C₈)-cycloalkyl,

where alkyl and cycloalkyl in turn may be substituted up to three times, independently of one another, by hydroxyl, (C₁-C₆)-alkoxy, mono- or di-(C₁-C₆)-alkylamino, optionally halogen-, trifluoromethyl- or (C₁-C₆)-alkoxy-substituted phenyl, biphenyl, naphtyl, optionally halogen-substituted 5- or 6-membered heteroaryl with up to three heteroatoms from the series N, O and/or S or optionally hydroxyl-substituted 5- to 10-membered heterocyclyl with up to three heteroatoms from the series N, O and/or S,

(C₆-C₁₀)-aryl, 5- to 10-membered heteroaryl with up to three heteroatoms from the series N, O and/or S or 5- or 6-membered heterocyclyl with up to three heteroatoms from the series N, O and/or S,

where aryl, heteroaryl and heterocyclyl in turn may be substituted up to three times, independently of one another, by phenyl, benzyl, morpholinyl, halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

or a radical of the formula —C(═O)—R⁴ or —SO₂—R⁴,

in which

R⁴ is hydrogen, (C₁-C₆)-alkyl,

which may in turn be substituted by hydroxyl, amino, phenyl, (C₆-C₁₀)-aryloxy, (C₁-C₆)-alkanoyloxy or (C₁-C₄)-alkoxy,

(C₆-C₁₀)-aryl, 5- to 10-membered heteroaryl with up to three heteroatoms from the series N, O and/or S, 5- to 10-membered heterocyclyl with up to three heteroatoms from the series N, O and/or S,

in which aryl, heteroaryl and heterocyclyl in turn may be substituted up to twice, independently of one another, by halogen, optionally hydroxyl-substituted (C₁-C₆)-alkyl, (C₁-C₆)-alkoxy, (C₁-C₆)-alkoxycarbonyl, phenyl or cyano,

(C₃-C₈)-cycloalkyl, (C₁-C₆)-alkoxycarbonyl or a radical of the formula —NR⁵R⁶ or —OR⁷,

in which

R⁵ and R⁶ are, independently of one another, hydrogen, (C₆-C₁₀)-aryl, adamantyl, (C₁-C₈)-alkyl,

whose chain may be interrupted by one or two oxygen atoms and which may be substituted up to three times by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms from the series N, O and/or S or by 5- to 10-membered heteroaryl with up to three heteroatoms from the series N, O and/or S,

(C₃-C₈)-cycloalkyl,

which may be substituted up to three times by (C₁-C₄)-alkyl, hydroxyl or oxo,

or 5- or 6-membered heterocyclyl with up to two heteroatoms from the series N, O and/or S, where N is substituted by hydrogen or (C₁-C₄)-alkyl,

or

R⁵ and R⁶ together with the nitrogen atom to which they are bonded form a 4- to 7-membered saturated heterocycle in which up to two ring carbon atoms are replaced by heteroatoms from the series N, O and/or S and which may be substituted by hydroxyl, oxo, aminocarbonyl, (C₁-C₆)-alkyl or (C₁-C₆)-alkoxy-(C₁-C₆)-alkyl,

and

R⁷is 5- or 6-membered heteroaryl with up to three heteroatoms from the series N, O and/or S,

which may be substituted up to twice, independently of one another, by (C₁-C₆)-alkyl, (C₁-C₆)-alkylthio or oxo,

(C₆-C₁₀)-aryl,

which may be substituted up to twice, independently of one another, by optionally (C₁-C₆)-alkoxycarbonyl- or carboxyl-substituted (C₁-C₆)-alkyl, (C₁-C₆)-alkoxy, di-(C₁-C₆)-alkylaminocarbonyl, mono- or di-(C₁-C₆)-alkylamino,

adamantyl, tetrahydronaphtyl, (C₁-C₈)-alkyl,

whose chain may be interrupted by one or two oxygen atoms and which may be substituted up to three times, independently of one another, by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms from the series N, O and/or S or by 5- to 10-membered heteroaryl with up to three heteroatoms from the series N, O and/or S,

(C₃-C₈)-cycloalkyl,

which may be substituted up to three times, independently of one another, by (C₁-C₄)-alkyl, hydroxyl or oxo,

or 5- to 10-membered heterocyclyl with up to two heteroatoms from the series N, O and/or S, where N is substituted by hydrogen or (C₁-C₄)-alkyl,

or

R¹ and R² together with the nitrogen atom to which they are bonded form a 5- to 10-membered saturated heterocycle with up to two further heteroatoms from the series N, O and/or S, which is optionally substituted up to twice, independently of one another, by benzyl or (C₆-C₁₀)-aryl which in turn may be substituted by halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

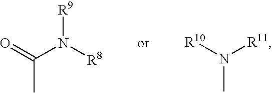

in which

R⁸ is a group of the formula

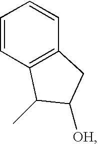

is (C₃-C₈)-cycloalkyl which may be substituted by (C₁-C₈)-alkyl, (C₆-C₁₀)-aryl, 5- to 10-membered heterocyclyl with up to three heteroatoms from the series N, O and/or S or 5- to 10-membered heteroaryl with up to three heteroatoms from the series N, O and/or S, where aryl, heterocyclyl and heteroaryl in turn may be substituted up to three times, independently of one another, by halogen, trifluoromethyl, cyano, nitro, hydroxyl, (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl or carboxyl,

or

is a methyl group,

which may be substituted up to three times, independently of one another, by hydrogen, trifluoromethyl, (C₃-C₈)-cycloalkyl, (C₁-C₈)-alkyl, whose chain may be interrupted by a sulfur atom or an S(O) or SO₂ group and which may be substituted up to twice, independently of one another, by hydroxyl, (C₁-C₆)-alkoxy, (C₁-C₆)-alkoxycarbonyl, halogen, cyano, nitro, trifluoromethoxy, oxo, amino, mono- or di-(C₁-C₆)-alkylamino, 5- or 6-membered heterocyclyl with up to three heteroatoms from the series N, O and/or S or carboxamide,

(C₁-C₆)-alkoxycarbonyl, (C₆-C₁₀)-aryl, benzyl, 5- to 10-membered heterocyclyl with up to three heteroatoms from the series N, O and/or S or 5- to 10-membered heteroaryl with up to three heteroatoms from the series N, O and/or S,

where aryl, benzyl, heterocyclyl and heteroaryl may be substituted up to three times, independently of one another, by halogen, trifluoromethyl, cyano, nitro, hydroxyl, optionally hydroxyl-substituted (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl, carboxyl, (C₁-C₆)-alkylcarbonylamino, (C₁-C₆)-alkoxycarbonylamino, aminocarbonyl, mono- or di-(C₁-C₆)-alkylaminocarbonyl which in turn may be substituted by (C₁-C₆)-alkoxy, or amidosulfone, mono- or di-(C₁-C₆)-alkylamidosulfone which in turn may be substituted by (C₁-C₆)-alkoxy,

R⁹ is hydrogen, (C₁-C₆)-alkoxy, (C₁-C₆)-alkyl- and/or phenyl-substituted amino, (C₁-C₆)-alkyl, (C₃-C₈)-cycloalkyl,

where alkyl and cycloalkyl in turn may be substituted up to three times, independently of one another, by hydroxyl or mono- or di-(C₁-C₆)-alkylamino,

(C₆-C₁₀)-aryl, 5- to 10-membered heteroaryl with up to three heteroatoms from the series N, O and/or S or 5- or 6-membered heterocyclyl with up to three heteroatoms from the series N, O and/or S,

where aryl, heteroaryl and heterocyclyl in turn may be substituted up to three times, independently of one another, by halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

or

R⁸ and R⁹ together with the nitrogen atom to which they are bonded form a 5- to 10-membered, optionally bicyclic heterocycle in which up to two ring carbon atoms are replaced by heteroatoms from the series N, O and/or S and which may be substituted up to four times, independently of one another, by hydroxyl, (C₁-C₆)-alkyl, (C₁-C₆)-alkoxy, hydroxy-(C₁-C₆)-alkyl, (C₁-C₆)-alkoxy-(C₁-C₆)-alkyl, oxo, amino or mono- or di-(C₁-C₆)-alkylamino,

R₁₀ is hydrogen, (C₁-C₆)-alkoxy, (C₁-C₆)-alkyl, (C₃-C₈)-cycloalkyl, where alkyl and cycloalkyl in turn may be substituted, independently of one another, up to three times by hydroxyl or mono- or di-(C₁-C₆)-alkylamino,

(C₆-C₁₀)-aryl, 5- to 10-membered heteroaryl with up to three heteroatoms from the series N, O and/or S or 5- or 6-membered heterocyclyl with up to three heteroatoms from the series N, O and/or S,

where aryl, heteroaryl and heterocyclyl may in turn be substituted, independently of one another, up to three times by halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

R¹¹ is a radical of the formula —C(═O)—R¹² or —SO₂—R¹²,

in which

R¹² is hydrogen, (C₁-C₆)-alkyl which may in turn be substituted by hydroxyl or (C₁-C₄)-alkoxy, or (C₆-C₁₀)-aryl, 5- to 10-membered heteroaryl with up to three heteroatoms from the series N, O and/or S, in which aryl and heteroaryl in turn may be substituted, independently of one another, by halogen, or (C₃-C₈)-cycloalkyl or a radical of the formula —NR¹³R¹⁴ or —OR¹⁵,

in which

R¹³ and R¹⁴ are, independently of one another, hydrogen, (C₆-C₁₀)-aryl, adamantyl, (C₁-C₈)-alkyl,

whose chain may be interrupted by one or two oxygen atoms and which may be substituted up to three times by hydroxyl, optionally halogen-, (C₁-C₆)-alkoxy- or amino-substituted phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms from the series N, O and/or S or by 5- to 10-membered heteroaryl with up to three heteroatoms from the series N, O and/or S,

(C₃-C₈)-cycloalkyl,

which may be substituted up to three times by (C₁-C₄)-alkyl, hydroxyl or oxo,

or 5- or 6-membered heterocyclyl with up to two heteroatoms from the series N, O and/or S, where N is substituted by hydrogen or (C₁-C₄)-alkyl,

or

R¹³ and R¹⁴ together with the nitrogen atom to which they are bonded form a 4- to 7-membered saturated heterocycle which may contain up to two further heteroatoms from the series N, O and/or S and is optionally substituted by hydroxyl, oxo, (C₁-C₆)-alkyl or (C₁-C₆)-alkoxy-(C₁-C₆)-alkyl,

R¹⁵ is (C₆-C₁₀)-aryl, adamantyl, (C₁-C₈)-alkyl,

whose chain may be interrupted by one or two oxygen atoms and which may be substituted up to three times, independently of one another, by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms from the series N, O and/or S or by 5- to 10-membered heteroaryl with up to three heteroatoms from the series N, O and/or S,

(C₃-C₈)-cycloalkyl,

which may be substituted up to three times, independently of one another, by (C₁-C₄)-alkyl, hydroxyl or oxo,

or 5- or 6-membered heterocyclyl with up to two heteroatoms from the series N, O and/or S, where N is substituted by hydrogen or (C₁-C₄)-alkyl,

and their salts, hydrates, hydrates of the salts and solvates.

Salts of the compounds according to the invention are physiologically acceptable salts of the substances according to the invention with mineral acids, carboxylic acids or sulfonic acids. Particularly preferred examples are salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid.

Salts may likewise be physiologically acceptable metal or ammonium salts of the compounds according to the invention. Particular preference is given to alkali metal salts (for example sodium or potassium salts), alkaline earth metal salts (for example magnesium or calcium salts), and ammonium salts which are derived from ammonia or organic amines such as, for example, ethylamine, di- or triethylamine, di- or triethanolamine, dicyclohexylamine, dimethylaminoethanol, arginine, lysine, ethylenediamine or 2-phenylethylamine.

The compounds according to the invention may, depending on the substitution pattern, exist in stereoisomeric forms which either are related as image and mirror image (enantiomers) or are not related as image and mirror image (diastereomers). The invention relates both to the enantiomers or diastereomers or respective mixtures thereof. The racemic forms can, just like the diastereomers, be separated into the stereoisomerically homogeneous components in a known manner.

In addition, the invention also encompasses prodrugs of the compounds according to the invention. The term prodrugs refers according to the invention to those forms of the compounds of the formula (I) which themselves may be biologically active or inactive but can be converted under physiological conditions into the corresponding biologically active form (for example by metabolism or solvolysis).

The terms “hydrates” and “solvates” refer according to the invention to those forms of the compounds of the formula (I) which form a molecular compound or a complex in the solid or liquid state through hydration with water or coordination with solvent molecules. Examples of hydrates are sesquihydrates, monohydrates, dihydrates or trihydrates. Equally suitable are the hydrates and solvates of salts of the compounds according to the invention.

Halogen stands for fluorine, chlorine, bromine and iodine. Chlorine or fluorine are preferred.

(C₁-C₈)-Alkyl stands for a straight-chain or branched alkyl radical with 1 to 8 carbon atoms. Examples which may be mentioned are: methyl, ethyl, n-propyl, isopropyl, n-butyl, isobutyl, tert-butyl, n-pentyl, n-hexyl and n-octyl. The corresponding alkyl groups with fewer carbon atoms, such as, for example, (C₁-C₆)-alkyl, (C₁-C₄)-alkyl and (C₁-C₃)-alkyl, are derived analogously from this definition. It is generally true that (C₁-C₃)-alkyl is preferred.

The meaning of the corresponding component of other more complex substituents is also derived from this definition, such as, for example, for mono- or di-alkylamino, hydroxyalkyl, alkylcarbonylamino, alkylaminocarbonyl, alkylamidosulfone, di-(C₁-C₆)-alkylaminocarbonyl or alkoxyalkyl.

(C₁-C₆)-Alkanoyloxy stands for an alkyl radical which has in the 1 position a doubly bonded oxygen atom and a singly bonded oxygen atom and is linked via the singly bonded oxygen atom in the 1 position. Examples which may be mentioned are: acetoxy, propionoxy, n-butyroxy, i-butyroxy, pivaloyloxy and n-hexanoyloxy.

(C₂-C₆)-Alkenyl stands for a straight-chain or branched alkenyl radical with 2 to 6 carbon atoms. A straight-chain or branched alkenyl radical with 2 to 4 carbon atoms is preferred. Examples which may be mentioned are: vinyl, allyl, isopropenyl and n-but-2-en-1-yl.

(C₁-C₈)-Cycloalkyl stands for a cyclic alkyl radical with 3 to 8 carbon atoms. Examples which may be mentioned are: cyclopropyl, cyclobutyl, cyclopentyl, cyclohexyl, cycloheptyl or cyclooctyl. The corresponding cycloalkyl groups with fewer carbon atoms, such as, for example, (C₃-C₆)-cycloalkyl, are derived analogously from this definition. Cyclopropyl, cyclopentyl and cyclohexyl are preferred.

(C₁-C₆)-Alkoxy stands for a straight-chain or branched alkoxy radical with 1 to 6 carbon atoms. Examples which may be mentioned are: methoxy, ethoxy, n-propoxy, isopropoxy, n-butoxy, isobutoxy, tert-butoxy, n-pentoxy and n-hexoxy. The corresponding alkoxy groups with fewer carbon atoms such as, for example, (C₁-C₄)-alkoxy or (C₁-C₃)-alkoxy, are derived analogously from this definition. It is generally true that (C₁-C₃)-alkoxy is preferred.

The meaning of the corresponding component of other more complex substituents is also derived from this definition, such as, for example alkoxycarbonyl, alkoxycarbonylamino or alkoxyalkyl.

(C₆-C₁₀)-Aryl stands for an aromatic radical with 6 to 10 carbon atoms. Examples which may be mentioned are: phenyl and naphthyl.

5- to 10-membered heteroaryl with up to 3 heteroatoms from the series N, O and/or S stands for a mono- or bicyclic heteroaromatic system which is linked via a ring carbon atom of the heteroaromatic system, where appropriate also via a ring nitrogen atom of the heteroaromatic system. Examples which may be mentioned are: pyridyl, pyrimidyl, pyridazinyl, pyrazinyl, thienyl, furyl, pyrrolyl, pyrazolyl, imidazolyl, triazolyl, thiazolyl, oxazolyl, oxdiazolyl, isoxazolyl, benzofuranyl, benzothienyl or benzimidazolyl. The corresponding heterocycles with fewer heteroatoms, such as, for example, with up to 2 heteroatoms from the series N, O and/or S, are derived analogously from this definition. It is generally true that 5- or 6-membered aromatic heterocycles with up to 2 heteroatoms from the series N, O and/or S, such as, for example, pyridyl, pyrimidyl, thiazolyl, oxazolyl and imidazolyl, are preferred.

The meaning of the corresponding component of other more complex substituents, such as, for example, heteroarylene, is also derived from this definition.

5- to 10-membered heterocyclyl with up to 3 heteroatoms from the series N, O and/or S stands for a saturated or partially unsaturated, mono- or bicyclic heterocycle which is linked via a ring carbon atom or a ring nitrogen atom. The corresponding heterocycles with fewer ring atoms, such as, for example, 5- or 6-membered heterocyclyl, are derived analogously from this definition. Examples which may be mentioned are: tetrahydrofuryl, pyrrolidinyl, pyrrolinyl, dihydropyridinyl, piperidinyl, piperazinyl, morpholinyl, thiomorpholinyl, azepinyl. It is generally true that 5- or 6-membered saturated heterocycles are preferred, especially piperidinyl, piperazinyl, morpholinyl and pyrrolidinyl.

Preference is given to compounds of the formula (I)

in which

M is a group —N(—R¹)— or an oxygen atom —O—,

A is a group —C(═O)— or —CH₂— or a chemical bond,

D is 5- or 6-membered heteroarylene with up to three heteroatoms from the series N, O and/or S or phenylene, each of which may be substituted up to twice, independently of one another, by halogen, hydroxyl, cyano, carboxyl, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

R¹ is hydrogen, benzyl (C₂-C₆)-alkenyl, (C₁-C₆)-alkyl,

where alkyl in turn may be substituted by (C₁-C₄)-alkoxy, phenyl, (C₃-C₈)-cycloalkyl or mono- or di-(C₁-C₄)-alkylamino,

phenyl or 5- or 6-membered heteroaryl with up to three heteroatoms from the series N, O and/or S,

where phenyl and heteroaryl in turn may be substituted up to twice, independently of one another, by halogen, trifluoromethyl, trifluoromethoxy, (C₁-C₄)-alkyl, (C₁-C₄)-alkoxy, (C₁-C₄)-alkoxycarbonyl, N-acetyl,N-methylamino or mono- or di-(C₁-C₄)-alkylamino,

R² is (C₁-C₆)-alkyl, (C₃-C₈)-cycloalkyl,

where alkyl and cycloalkyl in turn may be substituted up to twice, independently of one another, by (C₁-C₄)-alkoxy, mono- or di-(C₁-C₆)-alkylamino, optionally halogen-, trifluoromethyl- or (C₁-C₆)-alkoxy-substituted phenyl, biphenyl, naphtyl or optionally halogen-substituted 5- or 6-membered heteroaryl with up to three heteroatoms from the series N, O and/or S,

phenyl or 5- or 6-membered heteroaryl with up to two heteroatoms from the series N, O and/or S,

where aryl and heteroaryl in turn may be substituted up to twice, independently of one another, by phenyl, benzyl, morpholinyl, halogen, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino, or a radical of the formula —C(═O)—R⁴ or —SO₂—R⁴,

in which

R⁴ is hydrogen, methyl, ethyl,

each of which in turn may be substituted by hydroxyl, amino, phenyl, (C₆-C₁₀)-aryloxy, (C₁-C₆)-alkanoyloxy or (C₁-C₄)-alkoxy,

phenyl, 5- or 6-membered heteroaryl with up to two heteroatoms from the series N, O and/or S, 5- or 6-membered heterocyclyl with up to two heteroatoms from the series N, O and/or S,

in which phenyl, heteroaryl and heterocyclyl may in turn be substituted up to twice, independently of one another, by halogen, optionally hydroxyl-substituted (C₁-C₄)-alkyl, (C₁-C₄)-alkoxy, (C₁-C₄)-alkoxycarbonyl, phenyl or cyano,

(C₃-C₈)-cycloalkyl or a radical of the formula —NR⁵R⁶ or —OR⁷,

in which

R⁵ and R⁶ are, independently of one another, phenyl or (C₁-C₆)-alkyl, whose chain may be interrupted by an oxygen atom and which may be substituted up to twice by phenyl, trifluoromethyl, (C₃-C₆)-cycloalkyl or (C₁-C₆)-alkoxy,

or

R⁵ and R₆ together with the nitrogen atom to which they are bonded form a 5- to 7-membered saturated heterocycle in which one ring carbon atom is replaced by a heteroatom from the series N, O or S and which may be substituted by hydroxyl, oxo, (C₁-C₆)-alkyl or (C₁-C₂)-alkoxy-(C₁-C₂)-alkyl,

and

R⁷is 5- or 6-membered heteroaryl with up to three heteroatoms from the series N, O and/or S,

which may be substituted up to twice, independently of one another, by (C₁-C₆)-alkyl, (C₁-C₆)-alkylthio or oxo,

(C₆-C₁₀)-aryl,

which may be substituted up to twice, independently of one another, by optionally [lacuna] (C₁-C₆)-alkoxy, di-(C₁-C₆)-alkylaminocarbonyl, mono- or di-(C₁-C₆)-alkylamino,

tetrahydronaphtyl, (C₁-C₄)-alkyl,

whose chain may be interrupted by an oxygen atom and which may be substituted up to twice, independently of one another, by phenyl, trifluoromethyl, (C₃-C₆)-cycloalkyl or (C₁-C₆)-alkoxy,

(C₃-C₈)-cycloalkyl,

which may be substituted up to three times, independently of one another, by (C₁-C₄)-alkyl, hydroxyl or oxo,

or 5- or 6-membered heterocyclyl with up to two heteroatoms from the series N, O and/or S, where N is substituted by hydrogen or (C₁-C₄)-alkyl,

or

R¹ and R² together with the nitrogen atom to which they are bonded form a 5- or 6-membered saturated heterocycle with optionally one further heteroatom from the series N, O or S, which is optionally substituted up to twice, independently of one another, by benzyl or phenyl which in turn may be substituted by halogen, hydroxyl, cyano, nitro, trifluoromethyl, trifluoromethoxy, (C₁-C₄)-alkyl, (C₁-C₄)-alkoxy, (C₁-C₄)-alkoxycarbonyl or mono- or di-(C₁-C₆)-methylamino,

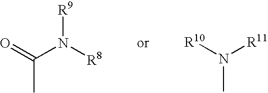

R³ is a group or

in which

R⁸ is a group of the formula

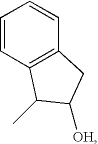

is (C₃-C₅)-cycloalkyl which may be substituted by phenyl or 5- or 6-membered heteroaryl with up to two heteroatoms from the series N, O and/or S,

where phenyl and heteroaryl in turn may be substituted up to twice, independently of one another, by halogen, trifluoromethyl, cyano, nitro, hydroxyl, (C₁-C₆)alkyl, (C₃-C₆)-cycloalkyl, (C₁-C₆)-alkoxy, amino, mono- or di-(C₁-C₂)-alkylamino,

or

is a methyl group,

which is substituted by hydrogen,

by trifluoromethyl, (C₃-C₆)-cycloalkyl or (C₁-C₄)-alkyl which may in turn be substituted by hydroxyl, (C₁-C₄)-alkoxy, halogen, cyano, trifluoromethoxy, amino, mono- or di-(C₁-C₄)-alkylamino, 5- or 6-membered heterocyclyl with up to two heteroatoms from the series N, O and/or S or carboxamide,

and by (C₆-C₁₀)-aryl or 5- to 10-membered heteroaryl with up to three heteroatoms from the series N, O and/or S,

where aryl and heteroaryl in turn may be substituted up to twice, independently of one another, by halogen, trifluoromethyl, cyano, nitro, hydroxyl, optionally hydroxyl-substituted (C₁-C₄)-alkyl, (C₃-C₆)-cycloalkyl, (C₁-C₄)-alkoxy, amino, mono- or di-(C₁-C₄)-alkylamino, (C₁-C₄)-alkoxycarbonyl, carboxyl, (C₁-C₄)-alkylcarbonylamino, (C₁-C₄)-alkoxycarbonylamino, aminocarbonyl, mono- or di-(C₁-C₄)-alkylaminocarbonyl, amidosulfone, mono- or di-(C₁-C₄)-alkylamidosulfone,

R⁹ is hydrogen or (C₁-C₂)-alkyl- and phenyl-substituted amino,

R¹⁰ is hydrogen,

R¹¹ is a radical of the formula —C(═O)—R¹²,

in which

R¹² is a radical of the formula —NR¹³R¹⁴,

in which

R¹³ is hydrogen,

R¹⁴ is a methyl group

which is substituted by hydrogen, methyl or ethyl and by phenyl which in turn may be substituted by halogen, (C₁-C₄)-alkoxy or amino,

and their salts, hydrates, hydrates of the salts and solvates.

Particular preference is given to compounds of the formula (I)

in which

M is a group —N(—R¹)— or an oxygen atom —O—,

A is a group —CH₂— or a chemical bond,

D is 5- or 6-membered heteroarylene with up to two heteroatoms from the series N, O and/or S or phenylene, each of which may be substituted up to twice, independently of one another, by halogen, trifluoromethyl, trifluoromethoxy, (C₁-C₄)-alkyl or (C₁-C₄)-alkoxy,

R¹ is hydrogen, phenyl, (C₂-C₄)-alkenyl or (C₁-C₄)-alkyl,

where alkyl in turn may be substituted by methoxy, (C₃-C₆)-cycloalkyl or mono- or dimethylamino,

R² is (C₁-C₄)-alkyl, (C₃-C₆)-cycloalkyl,

where alkyl and cycloalkyl in turn may be substituted up to twice, independently of one another, by methoxy, mono- or dimethylamino, optionally halogen-, trifluoromethyl- or methoxy-substituted phenyl, biphenyl, naphtyl or optionally halogen-substituted 5- or 6-membered heteroaryl with up to two heteroatoms from the series N, O and/or S,

phenyl or 5- or 6-membered heteroaryl with up to two heteroatoms from the series N, O and/or S,

where aryl and heteroaryl in turn may be substituted up to twice, independently of one another, by halogen, cyano, nitro, trifluoromethyl, trifluoromethoxy, (C₁-C₄)-alkyl, (C₁-C₄)-alkoxy or mono- or dimethylamino,

or a radical of the formula —C(═O)—R⁴,

in which

R⁴ is methyl,

which may in turn be substituted by phenyl, phenyloxy or (C₁-C₂)-alkoxy,

phenyl, 5- or 6-membered heteroaryl with up to two heteroatoms from the series N, O and/or S, 5- or 6-membered heterocyclyl with up to two heteroatoms from the series N, O and/or S,

in which phenyl, heteroaryl and heterocyclyl in turn may be substituted up to twice, independently of one another, by halogen, methoxy or (C₁-C₄)-alkoxycarbonyl,

(C₃-C₄)-cycloalkyl or a radical of the formula —OR⁷,

in which

R⁷ is 5- or 6-membered heteroaryl with up to two heteroatoms from the series N, O and/or S,

which may be substituted up to twice, independently of one another, by (C₁-C₄)-alkyl or methylthio,

phenyl,

which may be substituted up to twice, independently of one another, by optionally [lacuna] (C₁-C₄)-alkoxy, dimethylaminocarbonyl or mono- or dimethylamino,

or tetrahydronaphtyl,

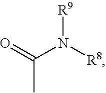

R³ is a group

in which

R⁸ is (C₃-C₅)-cycloalkyl which may be substituted by phenyl or 5- or 6-membered heteroaryl with up to two heteroatoms from the series N, O and/or S,

where phenyl and heteroaryl in turn may be substituted up to twice, independently of one another, by (C₁-C₄)-alkyl or (C₁-C₄)-alkoxy,

or

is a methyl group,

which is substituted by hydrogen,

by (C₁-C₃)-alkyl which may in turn be substituted by hydroxyl, (C₁-C₂)-alkoxy, amino or mono- or di-(C₁-C₄)-alkylamino,

and by phenyl or 5- to 10-membered heteroaryl with up to three heteroatoms from the series N, O and/or S,

where phenyl and heteroaryl in turn may be substituted up to twice, independently of one another, by halogen, trifluoromethyl, cyano, nitro, hydroxyl, optionally hydroxyl-substituted (C₁-C₂)-alkyl, (C₃-C₆)-cycloalkyl, (C₁-C₄)-alkoxy, amino, mono- or di-(C₁-C₄)-alkylamino, (C₁-C₄)-alkoxycarbonyl, (C₁-C₄)-alkylcarbonylamino, (C₁-C₄)-alkoxycarbonylamino, aminocarbonyl, mono- or di-(C₁-C₄)-alkylaminocarbonyl, amidosulfone, mono- or di-(C₁-C₄)-alkylamidosulfone,

R⁹ is hydrogen,

and their salts, hydrates, hydrates of the salts and solvates.

Preference is likewise given to compounds of the formula (I)

in which

M is a group —N(—R¹)—,

A is a group —C(═O)— or —CH₂— or a chemical bond,

D is 5- or 6-membered heteroarylene with up to three heteroatoms from the series N, O and/or S or phenylene, each of which may be substituted up to three times, independently of one another, by halogen, hydroxyl, cyano, carboxyl, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

R¹ is hydrogen, benzyl, (C₂-C₆)-alkenyl, (C₁-C₆)-alkyl, (C₃-C₈)-cycloalkyl,

where alkyl and cycloalkyl in turn may be substituted up to three times, independently of one another, by hydroxyl, phenyl or mono- or di-(C₁-C₆)-alkylamino,

(C₆-C₁₀)-aryl, 5- to 10-membered heteroaryl with up to three heteroatoms from the series N, O and/or S or 5- or 6-membered heterocyclyl with up to three heteroatoms from the series N, O and/or S,

where aryl, heteroaryl and heterocyclyl in turn may be substituted up to three times, independently of one another, by halogen, hydroxyl, oxo, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

R² is hydrogen, (C₁-C₆)-alkyl, (C₃-C₈)-cycloalkyl,

where alkyl and cycloalkyl in turn may be substituted up to three times, independently of one another, by hydroxyl, (C₁-C₆)-alkoxy, mono- or di-(C₁-C₆)-alkylamino, phenyl, biphenyl, naphtyl or optionally hydroxyl-substituted 5- to 10-membered heterocyclyl with up to three heteroatoms from the series N, O and/or S,

(C₆-C₁₀)-aryl, 5- to 10-membered heteroaryl with up to three heteroatoms from the series N, O and/or S or 5- or 6-membered heterocyclyl with up to three heteroatoms from the series N, O and/or S,

where aryl, heteroaryl and heterocyclyl in turn may be substituted up to three times, independently of one another, by phenyl, halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

or a radical of the formula —C(═O)—R⁴ or —SO₂—R⁴,

in which

R⁴ is hydrogen, (C₁-C₆)-alkyl which may in turn be substituted by hydroxyl or (C₁-C₄)-alkoxy, or (C₆-C₁₀)-aryl, 5- to 10-membered heteroaryl with up to three heteroatoms from the series N, O and/or S, in which aryl and heteroaryl in turn may be substituted, independently of one another, by halogen, or (C₃-C₈)-cycloalkyl or a radical of the formula —NR⁵R⁶ or —OR⁷,

in which

R⁵ and R⁶ are, independently of one another, hydrogen, (C₆-C₁₀)-aryl, adamantyl, (C₁-C₈)-alkyl, whose chain may be interrupted by one or two oxygen atoms and which may be substituted up to three times by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms from the series N, O and/or S or by 5- to 10-membered heteroaryl with up to three heteroatoms from the series N, O and/or S,

(C₃-C₈)-cycloalkyl which may be substituted up to three times by (C₁-C₄)-alkyl, hydroxyl or oxo, or 5- or 6-membered heterocyclyl with up to two heteroatoms from the series N, O and/or S, where N is substituted by hydrogen or (C₁-C₄)-alkyl,

or

R⁵ and R⁶ together with the nitrogen atom to which they are bonded form a 4- to 7-membered saturated heterocycle in which up to two ring carbon atoms are replaced by heteroatoms from the series N, O and/or S and which may be substituted by hydroxyl, oxo, aminocarbonyl, (C₁-C₆)-alkyl or (C₁-C₆)-alkoxy-(C₁-C₆)-alkyl,

and

R⁷is (C₆-C₁₀)-aryl, adamantyl, (C₁-C₈)-alkyl,

whose chain may be interrupted by one or two oxygen atoms and which may be substituted up to three times, independently of one another, by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms from the series N, O and/or S or by 5- to 10-membered heteroaryl with up to three heteroatoms from the series N, O and/or S,

(C₃-C₈)-cycloalkyl which may be substituted up to three times, independently of one another, by (C₁-C₄)-alkyl, hydroxyl or oxo, or 5- or 6-membered heterocyclyl with up to two heteroatoms from the series N, O and/or S, where N is substituted by hydrogen or (C₁-C₄)-alkyl,

or

R¹ and R² together with the nitrogen atom to which they are bonded form a 5- to 10-membered saturated heterocycle with up to two further heteroatoms from the series N, O and/or S, which is optionally substituted up to twice, independently of one another, by benzyl or (C₆-C₁₀)-aryl which in turn may be substituted by halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

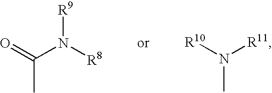

R³ is a group or

in which

R⁸ is a group of the formula

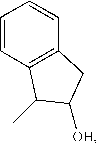

is (C₃-C₈)-cycloalkyl which may be substituted by (C₁-C₈)-alkyl, (C₆-C₁₀)-aryl, 5- to 10-membered heterocyclyl with up to three heteroatoms from the series N, O and/or S or 5- to 10-membered heteroaryl with up to three heteroatoms from the series N, O and/or S, where aryl, heterocyclyl and heteroaryl in turn may be substituted up to three times by halogen, trifluoromethyl, cyano, nitro, hydroxyl, (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl, carboxyl,

or

is a methyl group which may be substituted up to three times, independently of one another, by hydrogen, (C₃-C₈)-cycloalkyl, (C₁-C₈)-alkyl, whose chain may be interrupted by a sulfur atom or an S(O) or SO₂ group and which may be substituted up to twice by hydroxyl, (C₁-C₆)-alkoxy, (C₁-C₆)-alkoxycarbonyl, halogen, cyano, nitro, trifluoromethoxy, oxo, amino, mono- or di-(C₁-C₆)-alkylamino or carboxamide,

(C₁-C₆)-alkoxycarbonyl, (C₆-C₁₀)-aryl, benzyl, 5- to 10-membered heterocyclyl with up to three heteroatoms from the series N, O and/or S or 5- to 10-membered heteroaryl with up to three heteroatoms from the series N, O and/or S,

where aryl, benzyl, heterocyclyl and heteroaryl may be substituted up to three times by halogen, trifluoromethyl, cyano, nitro, hydroxyl, (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl, carboxyl, (C₁-C₆)-alkylcarbonylamino, (C₁-C₆)-alkoxycarbonylamino, aminocarbonyl, mono- or di-(C₁-C₆)-alkylaminocarbonyl which may in turn be substituted by (C₁-C₆)-alkoxy, or amidosulfone, mono- or di-(C₁-C₆)-alkylamidosulfone which may in turn be substituted by (C₁-C₆)-alkoxy,

R⁹ is hydrogen, (C₁-C₆)-alkoxy, (C₁-C₆)-alkyl, (C₃-C₈)-cycloalkyl,

where alkyl and cycloalkyl in turn may be substituted up to three times, independently of one another, by hydroxyl or mono- or di-(C₁-C₆)-alkylamino,

(C₆-C₁₀)-aryl, 5- to 10-membered heteroaryl with up to three heteroatoms from the series N, O and/or S or 5- or 6-membered heterocyclyl with up to three heteroatoms from the series N, O and/or S,

where aryl, heteroaryl and heterocyclyl in turn maybe substituted up to three times, independently of one another, by halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

or

R⁸ and R⁹ together with the nitrogen atom to which they are bonded form a 5- to 8-membered heterocycle in which up to two ring carbon atoms are replaced by heteroatoms from the series N, O and/or S and which may be substituted up to four times, independently of one another, by hydroxyl, (C₁-C₆)-alkyl, (C₁-C₆)-alkoxy, hydroxy-(C₁-C₆)-alkyl, (C₁-C₆)-alkoxy-(C₁-C₆)-alkyl, oxo, amino or mono- or di-(C₁-C₆)-alkylamino,

R¹⁰ is hydrogen, (C₁-C₆)-alkoxy, (C₁-C₆)-alkyl, (C₃-C₈)-cycloalkyl,

where alkyl and cycloalkyl may in turn be substituted, independently of one another, up to three times by hydroxyl or mono- or di-(C₁-C₆)-alkylamino,

(C₆-C₁₀)-aryl, 5- to 10-membered heteroaryl with up to three heteroatoms from the series N, O and/or S or 5- or 6-membered heterocyclyl with up to three heteroatoms from the series N, O and/or S,

where aryl, heteroaryl and heterocyclyl may in turn be substituted, independently of one another, up to three times by halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,

R¹¹ is a radical of the formula —C(═O)—R¹² or —SO₂—R¹²,

in which

R¹² is hydrogen, (C₁-C₆)-alkyl which may in turn be substituted by hydroxyl or (C₁-C₄)-alkoxy, or (C₆-C₁₀)-aryl, 5- to 10-membered heteroaryl with up to three heteroatoms from the series N, O and/or S, in which aryl and heteroaryl may in turn be substituted, independently of one another, by halogen, or (C₃-C₈)-cycloalkyl or a radical of the formula —NR¹³R¹⁴ or —OR¹⁵,

in which

R¹³ and R¹⁴ are, independently of one another, hydrogen, (C₆-C₁₀)-aryl, adamantyl, (C₁-C₈)-alkyl,

whose chain may be interrupted by one or two oxygen atoms and which may be substituted up to three times by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms from the series N, O and/or S or by 5- to 10-membered heteroaryl with up to three heteroatoms from the series N, O and/or S,

(C₃-C₈)-cycloalkyl which may be substituted up to three times by (C₁-C₄)-alkyl, hydroxyl or oxo, or 5- or 6-membered heterocyclyl with up to two heteroatoms from the series N, O and/or S, where N is substituted by hydrogen or (C₁-C₄)-alkyl,

or

R¹³ and R¹⁴ together with the nitrogen atom to which they are bonded form a 4- to 7-membered saturated heterocycle which may contain up to two further heteroatoms from the series N, O and/or S and is optionally substituted by hydroxyl, oxo, (C₁-C₆)-alkyl or (C₁-C₆)-alkoxy-(C₁-C₆)-alkyl,

R¹⁵ is (C₆-C₁₀)-aryl, adamantyl, (C₁-C₈)-alkyl,

whose chain may be interrupted by one or two oxygen atoms and which may be substituted up to three times, independently of one another, by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms from the series N, O and/or S or by 5- to 10-membered heteroaryl with up to three heteroatoms from the series N, O and/or S,

(C₃-C₈)-cycloalkyl which may be substituted up to three times, independently of one another, by (C₁-C₄)-alkyl, hydroxyl or oxo, or 5- or 6-membered heterocyclyl with up to two heteroatoms from the series N, O and/or S, where N is substituted by hydrogen or (C₁-C₄)-alkyl,

and their salts, hydrates, hydrates of the salts and solvates.

It is possible and likewise preferred in the compounds of the formula (I) for the radical

D to be phenylene, thiophendiyl, pyridinediyl, thiazoldiyl, pyrimidinediyl, imidazolediyl, pyrazolediyl, isoxazolediyl or oxazolediyl, each of which may be substituted up to three times, independently of one another, by halogen, hydroxyl, cyano, carboxyl, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino.

It is possible and particularly preferred in the compounds of the formula (I) for the radical

D to be 1,3- or 1,4-phenylene; 2,4- or 2,5-thiophendiyl or 2,4- or 2,5-pyridinediyl, each of which may be substituted up to three times, independently of one another, by halogen, hydroxyl, cyano, carboxyl, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino.

It is possible and very particularly preferred in the compounds of the formula (I) for the radical

D to be 1,3- or 1,4-phenylene; 2,4- or 2,5-thiophendiyl or 2,4- or 2,5-pyridinediyl, each of which may be substituted up to twice, independently of one another, by halogen, hydroxyl, cyano, carboxyl, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino.

In particular, it is possible and very particularly preferred in the compounds of the formula(I) for the radical

D to be 1,4-phenylene or 2,5-pyridinediyl, each of which may be substituted up to twice, independently of one another, by halogen, hydroxyl, cyano, carboxyl, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino.

It is possible and likewise preferred in the compounds of the formula (I) for the radical

R⁸ to be a methyl group which has as a substituent (C₆-C₁₀)-aryl or 5- to 10-membered heteroaryl with up to three heteroatoms from the series N, O and/or S,

where aryl and heteroaryl may be substituted up to three times by halogen, trifluoromethyl, cyano, nitro, hydroxyl, (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl, carboxyl, (C₁-C₆)-alkylcarbonylamino, (C₁-C₆)-alkoxycarbonylamino, aminocarbonyl, mono- or di-(C₁-C₆)-alkylaminocarbonyl, which may in turn be substituted by (C₁-C₆)-alkoxy, or amidosulfone, mono- or di-(C₁-C₆)-alkylamidosulfone, which may in turn be substituted by (C₁-C₆)-alkoxy,

and additionally may be substituted up to twice, independently of one another, by hydrogen, (C₃-C₈)-cycloalkyl, (C₁-C₈)-alkyl,

whose chain may be interrupted by a sulfur atom or an S(O) or SO₂ group and which may be substituted up to twice by hydroxyl, (C₁-C₆)-alkoxy, (C₁-C₆)-alkoxycarbonyl, halogen, cyano, nitro, trifluoromethoxy, oxo, amino, mono- or di-(C₁-C₆)-alkylamino or carboxamide,

(C₁-C₆)-alkoxycarbonyl, (C₆-C₁₀)-aryl, benzyl, 5- to 10-membered heterocyclyl with up to three heteroatoms from the series N, O and/or S or 5- to 10-membered heteroaryl with up to three heteroatoms from the series N, O and/or S,

where aryl, benzyl, heterocyclyl and heteroaryl may be substituted up to three times by halogen, trifluoromethyl, cyano, nitro, hydroxyl, (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl, carboxyl, (C₁-C₆)-alkylcarbonylamino, (C₁-C₆)-alkoxycarbonylamino, aminocarbonyl, mono- or di-(C₁-C₆)-alkylaminocarbonyl, which may in turn be substituted by (C₁-C₆)-alkoxy,

or amidosulfone, mono- or di-(C₁-C₆)-alkylamidosulfone, which may in turn be substituted by (C₁-C₆)-alkoxy.

It is likewise possible and particularly preferred in the compounds of the formula (I) for the radical

R⁸ to be a methyl group which has as a substituent hydrogen, has as a further substituent (C₆-C₁₀)-aryl or 5- to 10-membered heteroaryl with up to three heteroatoms from the series N, O and/or S, where aryl and heteroaryl may be substituted up to three times by halogen, trifluoromethyl, cyano, nitro, hydroxyl, (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl, carboxyl, (C₁-C₆)-alkylcarbonylamino, (C₁-C₆)-alkoxycarbonylamino, aminocarbonyl, mono- or di-(C₁-C₆)-alkylaminocarbonyl, which may in turn be substituted by (C₁-C₆)-alkoxy, or amidosulfone, mono- or di-(C₁-C₆)-alkylamidosulfone, which may in turn be substituted by (C₁-C₆)-alkoxy,

and is additionally substituted by hydrogen, (C₃-C₈)-cycloalkyl, (C₁-C₈)-alkyl,

whose chain may be interrupted by a sulfur atom or an S(O) or SO₂ group and which may be substituted up to twice by hydroxyl, (C₁-C₆)-alkoxy, (C₁-C₆)-alkoxycarbonyl, halogen, cyano, nitro, trifluoromethoxy, oxo, amino, mono- or di-(C₁-C₆)-alkylamino or carboxamide,

(C₁-C₆)-alkoxycarbonyl, (C₆-C₁₀)-aryl, benzyl, 5- to 10-membered heterocyclyl with up to three heteroatoms from the series N, O and/or S or 5- to 10-membered heteroaryl with up to three heteroatoms from the series N, O and/or S,

where aryl, benzyl, heterocyclyl and heteroaryl may be substituted up to three times by halogen, trifluoromethyl, cyano, nitro, hydroxyl, (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl, carboxyl, (C₁-C₆)-alkylcarbonylamino, (C₁-C₆)-alkoxycarbonylamino, aminocarbonyl, mono- or di-(C₁-C₆)-alkylaminocarbonyl, which may in turn be substituted by (C₁-C₆)-alkoxy,

or amidosulfone, mono- or di-(C₁-C₆)-alkylamidosulfone, which may in turn be substituted by (C₁-C₆)-alkoxy.

It is likewise possible and very particularly preferred in the compounds of the formula (1) for the radical

R⁸ to be a methyl group which has as a substituent hydrogen, has as a further substituent (C₆-C₁₀)-aryl or 5- to 10-membered heteroaryl with up to three heteroatoms from the series N, O and/or S, where aryl and heteroaryl may be substituted up to three times by halogen, trifluoromethyl, cyano, nitro, hydroxyl, (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl, carboxyl, (C₁-C₆)-alkylcarbonylamino, (C₁-C₆)-alkoxycarbonylamino, aminocarbonyl, mono- or di-(C₁-C₆)-alkylaminocarbonyl, which may in turn be substituted by (C₁-C₆)-alkoxy, or amidosulfone, mono- or di-(C₁-C₆)-alkylamidosulfone, which may in turn be substituted by (C₁-C₆)-alkoxy,

and is additionally substituted by hydrogen, (C₃-C₈)-cycloalkyl or (C₁-C₈)-alkyl,

whose chain may be interrupted by a sulfur atom or an S(O) or SO₂ group and which may be substituted up to twice by hydroxyl, (C₁-C₆)-alkoxy, (C₁-C₆)-alkoxycarbonyl, halogen, cyano, nitro, trifluoromethoxy, oxo, amino, mono- or di-(C₁-C₆)-alkylamino or carboxamide.

The compounds according to the invention of the formula (I) can be prepared by the following process, which comprises three different reaction sequences:

In one possible reaction sequence, compounds of the formula (II)

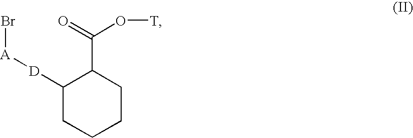

in which A and D have the meanings stated above, and T is (C₁-C₄)-alkyl, preferably methyl or tert-butyl,

are initially converted into compounds of the formula (IV)

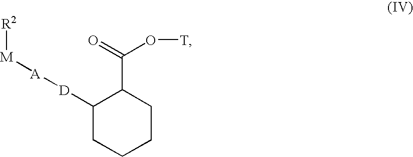

in which A, D, M, R² and T have the meanings stated above.

Various routes are possible for this, depending on the meaning of A and M.

1. In the case where A is a CH₂ group, the corresponding compounds of the formula (IIa)

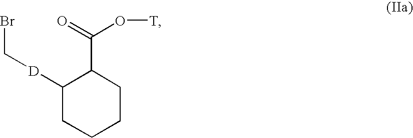

in which D and T have the meanings stated above,

are reacted with compounds of the formula (III) or (IIId)

HNR¹R² (III)

HO—R² (IIId),

in which R¹ and R² have the meanings stated above,

in a solvent, where appropriate in the presence of a base, to give compounds of the formula (IV).

2. In the case where A is a CO group and M is a group —N(—R¹)—, the compounds of the formula (IIa) described above are initially converted by reaction with an oxidizing agent in a solvent into the compounds of the formula (IIIb)

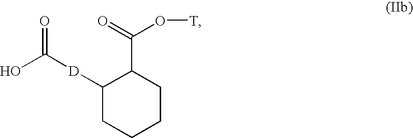

in which D and T have the meanings stated above,

and the compounds of the formula (IIb) obtained in this way are subsequently reacted with the compounds of the formula (III) described above in a solvent, where appropriate in the presence of a base and/or of a condensing agent, to give the corresponding compounds of the formula (IV).

3. In the case where A is a bond and M is a group —N(—R¹)—, the compounds of the formula (IIb) described above are reacted to give compounds of the formula (IIc)

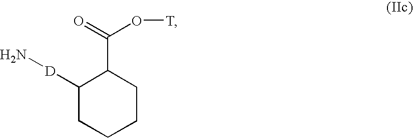

in which D and T have the meaning stated above,

in a conventional rearrangement reaction such as, for example, by the Curtius or Hofman method, and subsequently

either

reacted in a solvent, where appropriate in the presence of a base, successively in any sequence with the compounds of the formula (IIIa) and (IIIb)

R¹—V (IIIa)

R²—V′ (IIIb),

in which R¹ and R² have the meanings stated above, and V and V′ are suitable leaving groups such as, for example, mesylate, tosylate or halogen, preferably chlorine or bromine,

or

converted by reductive amination in a solvent using a suitable reducing agent with compounds of the formula (IIIc)

R¹⁶—CHO (IIIc)

in which R¹⁶ is phenyl, (C₂-C₅)-alkenyl or (C₁-C₅)-alkyl, which may be substituted up to three times, independently of one another, by hydroxyl, phenyl or mono- or di-(C₁-C₆)-alkylamino,

into compounds of the formula (IVa)

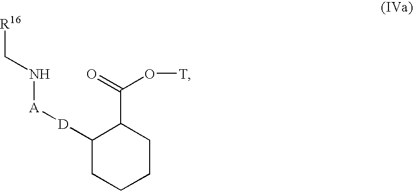

in which A, D, R¹⁶ and T have the meanings stated above,

and subsequently reacted in a solvent, where appropriate in the presence of a base, with compounds of the formula (IIIb)

R²—V′ (IIIb)

in which R² and V′ have the meanings stated above,

to give the corresponding compounds of the formula (IV).

The compounds of the formula (IV) prepared in this way are then converted in a solvent, where appropriate in the presence of an acid or base, into compounds of the formula (V)

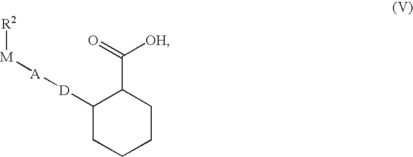

in which A, D, M and R² have the meanings stated above.

Two routes are possible for further introduction of the radical R³.

Either compounds of the formula (V) are reacted with compounds of the formula (VI)

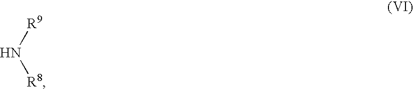

in which R⁸ and R⁹ have the meanings stated above,

in a solvent, where appropriate in the presence of a base and/or of a condensing agent. The radical R³ in the compounds of the formula (I) obtained in this way is —CONR⁸R⁹.

Alternatively, the compounds of the formula (V) are reacted in a conventional rearrangement reaction such as, for example, by the Curtius or Hofman method. The compounds of the formula (VII) obtained in this way

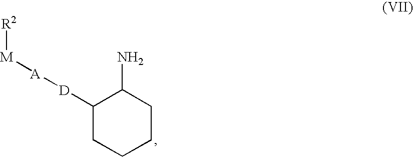

in which A, D, M and R² have the meanings stated above, are finally

either

reacted successively in any sequence with the compounds of the formula (VIIIa) and (VIIIb)

R¹⁰—W (VIIIa)

R¹¹—W′(VIIIb)

in which R¹⁰ and R¹¹ have the meanings stated above, and W and W′ are suitable leaving groups such as, for example, halogen, preferably chlorine or bromine,

in a solvent, where appropriate in the presence of a base,

or

converted by reductive amination in a solvent using a suitable reducing agent with compounds of the formula (VIIIc)

R¹⁷—CHO (VIIIc)

in which R¹⁷ is (C₁-C₅)-alkyl which may be substituted up to three times, independently of one another, by hydroxyl or mono- or di-(C₁-C₆)-alkylamino,

into compounds of the formula (Ia)

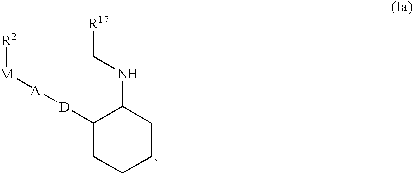

in which A, D, M, R² and R¹⁷ have the meanings stated above,

and subsequently reacted in a solvent, where appropriate in the presence of a base, with compounds of the formula (VIIIb)

R¹¹—W′ (VIIIb)

in which R¹¹ and W′ have the meanings stated above.

The radical R³ in the compounds of the formula (I) obtained in this way is —NR¹⁰R¹¹ or —N(CH₂R¹⁷)R¹¹.

In a second possible reaction sequence, the compounds of the formula (IIa) described above are firstly converted by reaction with an oxidizing agent in a solvent into the compounds of the formula (IId)

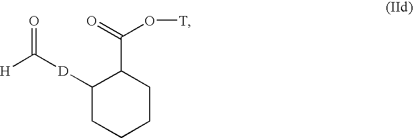

in which D and T have the meanings stated above. Subsequent reaction in a solvent, where appropriate in the presence of an acid or base, gives compounds of the formula (IX)

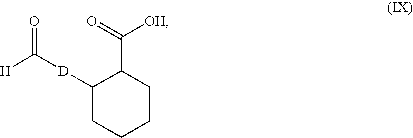

in which D has the meaning stated above. Reaction in a solvent, where appropriate in the presence of a base and/or of a condensing agent, with compounds of the formula (VI)

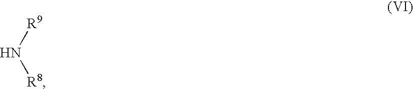

in which R⁸ and R⁹ have the meanings stated above,

then affords compounds of the formula (X)

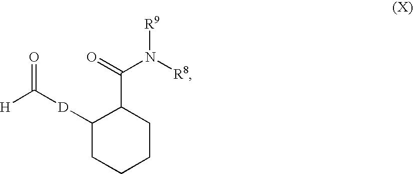

in which D, R⁸ and R⁹ have the meanings stated above.

Reductive amination of compounds of the formula (X) in a solvent using a suitable reducing agent with compounds of the formula (XI)

R¹—NH₂ (XI),

in which R¹ has the meaning stated above,

results in compounds of the formula (XII)

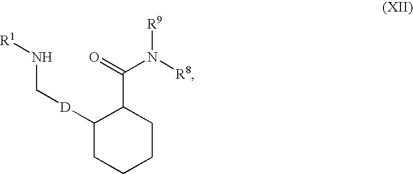

in which D, R¹, R⁸ and R⁹ have the meanings stated above.

Alternatively, compounds of the formula (XII) can be prepared by firstly reacting compounds of the formula (IIa), where appropriate in the presence of an acid or base, to give compounds of the formula (XVI)

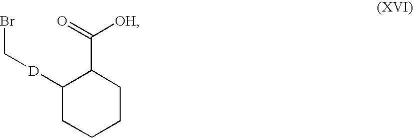

in which D has the meaning stated above. Subsequent reaction in a solvent, where appropriate in the presence of a base and/or of a condensing agent, with compounds of the formula (VI)

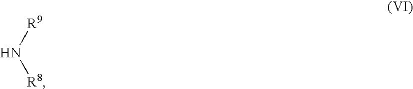

in which R⁸ and R⁹ have the meanings stated above,

then affords compounds of the formula (XVII)

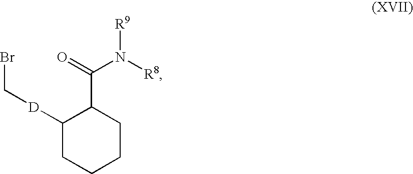

in which D, R⁸ and R⁹ have the meanings stated above,

which are then converted either

by reaction with compounds of the formula (XI) in a solvent, where appropriate in the presence of a base,

or

by nucleophilic substitution of the bromide by reaction with sodium azide and subsequent reduction of the azido group to the corresponding amino group

into compounds of the formula (XII).

Introduction of the radical R can finally take place in various ways, by either reacting compounds of the formula (XII)

1. by reaction with a phosgene equivalent such as, for example, trichloromethyl chloroformate in a solvent and subsequent reaction in a solvent, where appropriate in the presence of a base, with compounds of the formula (XIII)

HNR⁵R⁶ (XIII)

in which R and R6 have the meanings stated above,

or

2. by reaction in a solvent, where appropriate in the presence of a base, with compounds of the formula (XIV)

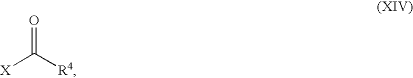

in which X is a leaving group such as, for example, the corresponding symmetric anhydride or a halogen, preferably chlorine, and R⁴ has the meaning stated above with the exception of NR⁵R⁶,

or

3. by reaction in a solvent, where appropriate in the presence of a base, with compounds of the formula (XV)

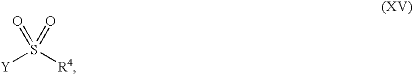

in which Y is a leaving group such as, for example, a halogen, preferably chlorine, and R⁴ has the meaning stated above,

to give compounds of the formula (I),

in which

A is a CH₂ group,

M is a group —N(—R¹)—,

R² is a radical —CO—R⁴ or —SO₂—R⁴ and

R³ is a radical —CONR⁸R⁹.

In a third possible reaction sequence, compounds of the formula (XVIII)

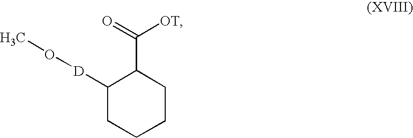

in which D and T have the meanings stated above,

are initially converted in a solvent, where appropriate in the presence of an acid or base, into compounds of the formula (XIX)

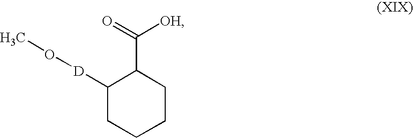

in which D has the meanings stated above,

and subsequently reacted with compounds of the formula (VI)

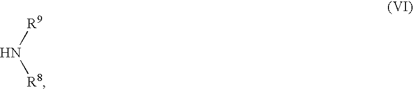

in which R⁸ and R⁹ have the meanings stated above,

in a solvent, where appropriate in the presence of a base and/or of a condensing agent, to give compounds of the formula (XX)

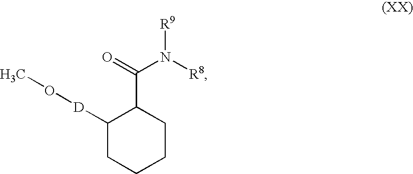

in which D, R⁸ and R⁹ have the meanings stated above. The methyl protective group is subsequently eliminated by reaction with boron tribromide. Reaction with compounds of the formula (IIIb)

R²—V′ (IIIb),

in which R² and V′ have the meanings stated above,

results in compounds of the formula (I) in which

A is a chemical bond and

M is an oxygen atom —O—.

The compounds of the formula (I) obtained in this way can, where appropriate, subsequently be converted by reaction for example with an acid into the corresponding salts.

Preparation of the compounds of the corresponding diastereomeric and enantiomeric forms takes place correspondingly, in particular either using enantiomerically or diastereomerically pure starting materials or else by subsequent separation of the formed racemates by conventional methods (for example racemate resolution, chromatography on chiral columns etc.).

The process according to the invention can be illustrated by way of example by the following formula diagrams:

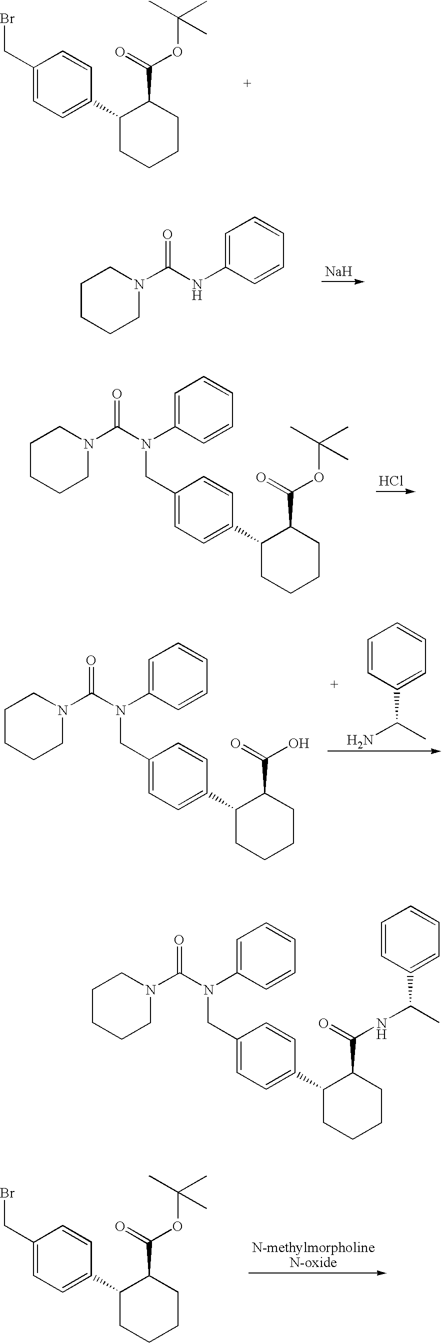

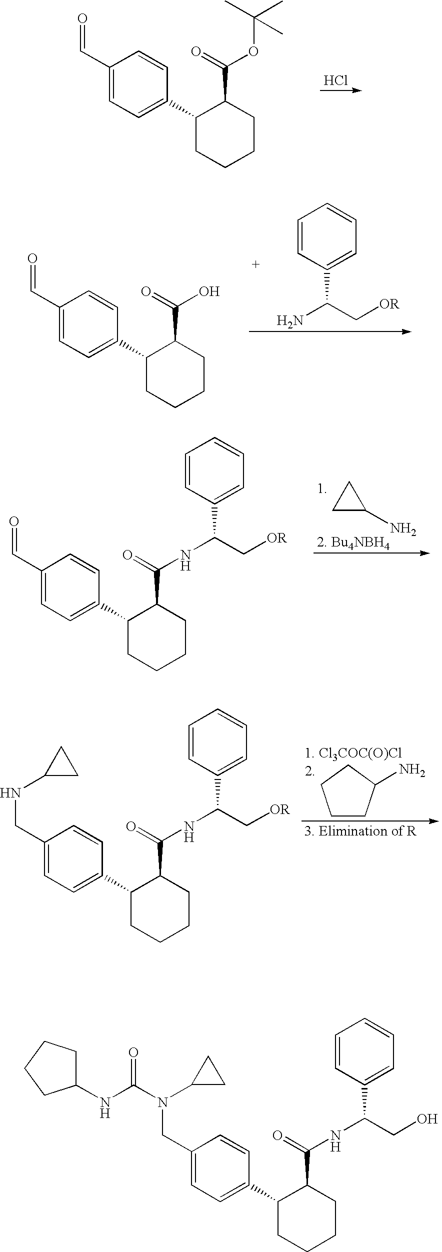

R is an oxygen protective group or a solid-phase resin

The process according to the invention is generally carried out under atmospheric pressure. However, the process can also be carried out under superatmospheric or subatmospheric pressure (for example in a range from 0.5 to 5 bar).

The reactions generally take place in a temperature range between −75° C. and +150° C., in particular between 0° C. and +80° C.

Suitable solvents for the process are conventional organic solvents which are not changed under the reaction conditions. These include ethers such as diethyl ether, dioxane, tetrahydrofuran, glycol dimethyl ether, or hydrocarbons such as benzene, toluene, xylene, hexane, cyclohexane or petroleum fractions, or halogenated hydrocarbons such as dichloromethane, trichloromethane, tetrachloromethane, dichloroethylene, trichloroethylene or chlorobenzene, or other solvents such as ethyl acetate, pyridine, dimethyl sulfoxide, dimethylformamide, N,N′-dimethylpropyleneurea (DMPU), N-methylpyrrolidone (NMP), acetonitrile, acetone, nitromethane or mixtures thereof.

Bases which can be employed for the process according to the invention are, in general, inorganic or organic bases. These include, preferably, alkali metal hydroxides such as, for example, sodium hydroxide or potassium hydroxide, alkaline earth metal hydroxides such as, for example, barium hydroxide, alkali metal carbonates such as sodium carbonate, potassium carbonate or cesium carbonate, alkaline earth metal carbonates such as calcium carbonate, or alkali metal or alkaline earth metal alcoholates such as sodium or potassium methanolate, sodium or potassium ethanolate or potassium tert-butoxide, or organic amines such as triethylamine, or heterocycles such as 1,4-diazabicyclo[2.2.2]octane (DABCO), 1,8-diazabicyclo[5.4.0]undec-7-ene (DBU), 1,5-diazabicyclo[4.3.0]non-5-ene (DBN), pyridine, diaminopyridine, N-methylpiperidine or N-methylmorpholine. It is also possible to employ alkali metals such as sodium or their hydrides such as sodium hydride as bases.

Auxiliaries preferably employed for the amide formation are conventional condensing agents such as carbodiimides, for example N,N′-diethyl-, N,N,′-dipropyl-, N,N′-diisopropyl-, N,N′-dicyclohexylcarbodiimde, N-(3-dimethylaminoisopropyl)-N′-ethylcarbodiimide hydrochloride (EDC) or carbonyl compounds such as carbonyldiimidazole or 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline or propanephosphonic anhydride or isobutyl chloroformate or bis(2-oxo-3-oxazolidinyl)phosphoryl chloride or benzotriazolyloxytri(dimethylamino)phosphonium hexafluorophosphate or O-(benzotriazol-1-yl)-N,N,N′,N′-tetramethyluronium hexafluorophosphate (HBTU) or O-(benzotriazol-1-yl)-N,N,N′,N′-tetramethyluronium tetrafluoroborate (TBTU) or 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU) or 1-hydroxybenzotriazole, and bases employed in the amide formation are alkali metal carbonates, for example sodium or potassium carbonate, or bicarbonate, or organic bases such as trialkylamines, for example triethylamine, N-methylmorpholine (NMM), 4-dimethylaminopyridine (DMAP), N-methylpiperidine or diisopropylethylamine (DIEA).

Reaction of compounds (IIa) with compounds (III) to give compounds (IV) preferably takes place in acetonitrile as solvent using potassium carbonate as base at a reaction temperature between 50° C. and 70° C. or in dimethylformamide as solvent using sodium hydride as base at room temperature. Reaction of compounds (IIa) with compounds (IIId) by contrast preferably takes place in dimethylformamide or tetrahydrofuran as solvent using sodium hydride as base.

Oxidation of compounds (IIa) to compounds (IIb) preferably takes place in dimethyl sulfoxide as solvent using sodium nitrite/acetic acid as oxidizing agent at room temperature.

The amide formation in the reaction of compounds (IIb) with compounds (III) to give compounds (IV) preferably takes place in dimethylformamide as solvent using EDC, HOBT and/or TBTU as auxiliaries and DMAP, DIEA or NMM as base at room temperature.

Rearrangement of compounds (IIb) to compounds (IIc) preferably takes place under the usual reaction conditions of a Hofman, Curtius or Lossen degradation. The reaction preferably takes place using diphenylphosphoryl azide (DPPA) in dioxane as solvent at room temperature or in n-hexane under reflux to prepare the isocyanate intermediate product. Subsequent hydrolysis of the isocyanate preferably takes place with potassium hydroxide as base in acetonitrile as solvent at room temperature.

Reaction of compounds (IIc) with compounds (IIIa) and (IIIb) to give compounds (IV) can take place in any sequence. It takes place, just like the reaction of compounds (XX) with compounds (IIIb), preferably in acetonitrile as solvent using potassium carbonate as base at a reaction temperature between 50° C. and 70° C. or in dimethylformamide as solvent using sodium hydride as base at room temperature.

The first step of the reductive amination of compounds (IIc) with compounds (IIIc) to give compounds (IVa) preferably takes place in a mixture of trimethyl orthoformate and dichloromethane as solvent or in methanol with acetic acid catalysis at room temperature. The subsequent reduction preferably takes place using tetrabutylammoniun borohydride or sodium cyanoborohydride as reducing agent in dimethylformamide as solvent at room temperature.

Reaction of compounds (IVa) with compounds (IIIb) to give compounds (IV) preferably takes place in acetonitrile as solvent using potassium carbonate as base at a reaction temperature between 50° C. and 70° C. or in dimethylformamide as solvent using sodium hydride as base at room temperature.

Hydrolysis of compounds (IV) to compounds (V) or of compounds (XVIII) to compounds (XIX) preferably takes place in dichloromethane as solvent using trifluoroacetic acid as acid at room temperature or in dioxane as solvent using hydrochloric acid at room temperature.

The amide formation in the reaction of compounds (V), or compounds (XIX), with compounds (VI), or compounds (XX), to give compounds (I), in which the radical R³ is —CONR⁸R⁹, preferably takes place in dimethylformamide as solvent using EDC, HOBT and/or TBTU as auxiliaries and DMAP, DIEA or NMM as base at room temperature.

Rearrangement of compounds (V) to compounds (VII) preferably takes place under the usual reaction conditions of a Hofman, Curtius or Lossen degradation. The reaction preferably takes place using diphenylphosphoryl azide (DPPA) in dioxane as solvent at room temperature or in n-hexane under reflux to prepare the isocyanate intermediate product. Subsequent hydrolysis of the isocyanate preferably takes place with potassium hydroxide as base in acetonitrile as solvent at room temperature.

Reaction of compounds (VII) with compounds (VIIIa) and (VIIIb) to give compounds (I) in which radical R³ is —NR¹⁰R¹¹ can take place in any sequence, preferably in acetonitrile as solvent using potassium carbonate as base at a reaction temperature between 50° C. and 70° C. or in dimethylformamide as solvent using sodium hydride as base at room temperature.

The first step of the reductive amination of compounds (VII) with compounds (VIIIc) to give compounds (Ia) preferably takes place in a mixture of trimethyl orthoformate and dichloromethane as solvent or in methanol with acetic acid catalysis at room temperature. The subsequent reduction preferably takes place using tetrabutylammoniun borohydride or sodium cyanoborohydride as reducing agent in dimethylformamide as solvent at room temperature.

Reaction of compounds (Ia) with compounds (VIIIb) to give compounds (I) preferably takes place in acetonitrile as solvent using potassium carbonate as base at a reaction temperature between 50° C. and 70° C. or in dimethylformamide as solvent using sodium hydride as base at room temperature.

Oxidation of compounds (IIa) to compounds (IId) preferably takes place using N-methylmorpholine N-oxide as oxidizing agent in acetonitrile as solvent at room temperature.

Hydrolysis of compounds (IId) to compounds (IX) or of compounds (IIa) to compounds (XVI) preferably takes place in dichloromethane as solvent using trifluoroacetic acid as acid at room temperature or in dioxane as solvent using hydrochloric acid at room temperature.

The amide formation in the reaction of compounds (IX), or compounds (XVI), with compounds (VI), or compounds (XVII), to give compounds (X) preferably takes place in dimethylformamide as solvent using EDC, HOBT and/or TBTU as auxiliaries and DMAP, DIEA or NMM as base at room temperature.

The first step of the reductive amination of compounds (X) with compounds (XI) to give compounds (XII) preferably takes place in a mixture of trimethyl orthoformate and dichloromethane as solvent or in methanol with acetic acid catalysis at room temperature. The subsequent reduction preferably takes place using tetrabutylammonium borohydride or sodium cyanoborohydride as reducing agent in dimethylformamide as solvent at room temperature.

Reaction of compounds (XII) with phosgene equivalents, preferably with trichloromethyl chloroformate, preferably takes place in dichloromethane as solvent at 0° C. Subsequent reaction with amines of the formua (XIII) preferably takes place in dimethylformamide as solvent in the presence of diisopropylethylamine as base at room temperature.

Reaction of compounds (XII) with compounds (XIV) or compounds (XV) preferably takes place in dichloromethane as solvent in the presence of diisopropylethylamine as base between room temperature and 50° C.

The compounds of the formulae (II), (IIa), (III), (IIIa), (IIIb), (IIIc), (VI), (VIIIa), (VIIIb), (XI), (XIII), (VIIIc), (XIV), (XV) and (XVIII) are known or can be prepared by conventional methods (compare EP-A-0 725 061, EP-A-0 725 064, EP-A-0 581 003, EP-A-0 611 767.

The compounds according to the invention of the formula (I) surprisingly show a valuable range of pharmacological effects which could not have been predicted.

The compounds according to the invention act as modulators, in particular as openers, of “BigK” channels. They can be used to produce medicaments for the prevention and/or treatment [lacuna] of the cardiovascular system such as, for example, of high blood pressure, heart failure and ischemia-related peripheral and cardiovascular disorders, especially for the acute and chronic treatment of ischemic disorders of the cardiovascular system, such as, for example, of coronary heart disease, of stable and unstable angina pectoris, of peripheral and arterial occlusive diseases, of thrombotic vascular occlusions, of myocardial infarction and of reperfusion damage.

In addition, owing to their potential for enhancing angiogenesis they are particularly suitable for permanent therapy of all occlusive diseases.

The compounds according to the invention can moreover be employed alone or in combination with other medicaments in oral or intravenous administration for the prevention and/or treatment of cerebrovascular disorders such as cerebral ischemia, stroke, reperfusion damage, brain trauma, edemas, seizures, epilepsy, respiratory arrest, cardiac arrest, Reye's syndrome, cerebral thrombosis, embolism, tumors, hemorrhages, encephalomyelitis, hydroencephalitis, spinal cord injuries, post operative brain damage, injuries to the retina or optic nerve after glaucoma, ischemia, hypoxia, edema or trauma, and in the treatment of schizophrenia, sleep disorders and acute and/or chronic pain, and neurodegenerative disorders, especially for the treatment of cancer-induced pain and chronic neuropathic pain, such as, for example, associated with diabetic neuropathy, post-therpeutic neuralgia, peripheral nerve damage, central pain (for example as consequence of cerebral ischemia) and trigeminal neuralgia and other chronic pain such as, for example, lumbago, back pain (lower back pain) or rheumatic pain.

The compounds according to the invention are additionally suitable for the prevention and/or treatment of disorders of the urogenital tract such as urinary incontinence, prostate hypertrophy, renal and urinary calculi, erectile dysfunction and sexual dysfunction.

A Assessment of the Physiological Activity

In Vitro Test Model for Testing “BigK Modulators”

CHO cells were stably transfected by electroporation with the human cDNA of the alpha subunit of the BigK channel. The cells were cultured in MEM alpha medium with 10% FCS.

1. Measurements of the Rubidium Efflux

Under physiological conditions, most potassium channels are very selective pores for potassium. In fact, these potassium channels, including BigK, also allow rubidium through to a comparable extent. However, because rubidium occurs in small amounts it has no physiological significance in this connection. Besides the widely used method of detecting radioactive rubidium, a very strong β and γ emitter, there is also the possibility of detecting and quantifying the rubidium flux through potassium channels nonradioactively (ion-channel characterization by atomic absorption, cf. DE-A44 33 261). After loading of the cells to be investigated, in which the intracellular potassium is very substantially replaced by rubidium, it is possible for rubidium to be detected quantitatively in the extracellular solution at various times. At the start of the experiment, the extracellular solution is nominally rubidium-free but contains potassium. Rubidium can escape from the loaded cells through nonspecific conductances and active transporters such as the sodium/potassium ATPase into the supernatant and thus lead to a certain nonspecific background. However, activation of potassium channels leads to a marked increase in the rubidium concentration over this background with time. The comparatively larger increase in the rubidium concentration in the cell supernatant indicates, as does the decrease associated therewith, in the intracellular rubidium concentration, a quantifiable activation of a potassium channel. Intracellular rubidium is quantified by treating the cells with a lysis buffer. In order to be able to detect BigK activity satisfactorily under high-throughput conditions, a CHO cell with very low potassium conductance was selected and stable expression of numerous functional BigK channels was induced. The rubidium concentration in the various solutions is routinely measured using an atomic absorption spectrometer (Unicam 939, Offenbach, Germany).

2. Patch Clamp Investigations

48 h after seeding of the cells on poly-D-lysine (100 μg/ml)-coated glass cover slips (30-50% confluence), the BigK channel currents were measured using fire-polished glass microelectrodes (R=2 to 5 MOhm) with a 150 mM intracellular and 5 mM extracellular potassium concentration. The potassium current was recorded with test potentials of −100 mV to +80 mV (pulse duration 100 ms). Activation of the BigK channel currents by the test substance was investigated in a concentration range from 10⁻⁸ to 10⁻⁵ M.

3. Langendorff Rat Heart

The heart is rapidly removed after opening the chest cavity of anesthetized rats and is introduced into a conventional Langendorff apparatus. The coronary arteries are subjected to constant-volume (10 ml/min) perfusion, and the perfusion pressure arising thereby is recorded via an appropriate pressure transducer. A decrease in the perfusion pressure in this arrangement corresponds to a relaxation of the coronary arteries. At the same time, the pressure developed by the heart during each contraction is measured via a balloon introduced into the left ventricle, and a further pressure transducer. The rate at which the isolated heart beats is found by calculation from the number of contractions per unit time.

TABLE 1
Biological activity of selected compounds in the Langendorff rat heart

| | Decrease in the perfusion | |
|---|---|---|
| Ex. No. | pressure from: | |
| 1 | 1 | μM |
| 7 | 0.1 | μM |
| 21 | 0.001 | μtM |
| 40 | 0.01 | μM |
| 80 | 1 | μM |
| 123 | 0.01 | μM |
| 168 | 0.01 | μM |
| 177 | 0.01 | μM |
| 210 | 0.001 | μM |

In Vivo Test Model for Testing “BigK Modulators”

4. Hemodynamic Effects in Dogs

Adult mongrel dogs (20-30 kg) are initially anesthetized with a combination of Trapanal and Alloferin. The anesthesia is maintained by infusion of a mixture of fentanyl, Alloferin and Dihydrobenzpyridyl. The animals are intubated [lacuna] ventilated with a 1:5 mixture of O₂/N₂O using a ventilating pump with about 16 breaths per min and a volume of 18-24 ml/kg. The body temperature is maintained at 38° C.±0.1° C. The arterial blood pressure is measured via a catheder in the femoral artery. A thoracotomy is performed at the fifth intercostal on the left side. The lung is pushed back and fixed, and the pericardium is incised. A proximal section of the LAD distal to the first diagonal branch is exposed and a calibrated electromagnetic flow probe (for example Gould Statham, model SP7515) is placed around the vessel and connected to a flow meter (for example Statham, model SP-2202). A mechanical occluder is placed distal to the flow probe so that there are no branches between the flow probe and occluder.

Blood sampling and substance administration are carried out through a catheder in the femoral vein. A peripheral ECG is recorded with subcutaneously fixed needles. A microtip pressure manometer (for example Millar model PC-350) is pushed through the left atrium in order to measure the left ventricular pressure. Measurement of the heart rate is triggered by the R wave of the ECG. The hemodynamic parameters and the coronary flow are recorded by a multi-channel recorder throughout the experiment.

5. Effect on the Mean Blood Pressure of Conscious, Spontaneously Hypertensive Rats

Continuous 24-hour measurements of blood pressure were carried out on freely moving, spontaneously hypertensive female rats (MOL:SPRD) weighing 200-250 g. For this purpose, pressure transducers (Data Sciences Inc., St. Paul, Minn., USA) had been implanted chronically in the descending abdominal aorta below the renal artery of the animals, and the transmitter connected thereto had been fixed in the abdominal cavity.

The animals were housed singly in type III cages positioned for the individual reception stations and were adapted to a 12-hour light/dark rhythm. Water and feed were available ad libitum.

To acquire the data, the blood pressure of each rat was recorded for 10 seconds every 5 minutes. The measurements were combined in each case for a period of 15 minutes, and the mean was calculated from these values.

The test compounds were dissolved in a mixture of Transcutol (10%), Cremophor (20%), H₂O (70%) and administered orally by gavage in a volume of 2 ml/kg of body weight. The test doses were between 0.3-30 mg/kg of body weight.

6. Measurements of Blood Pressure on Anesthetized Rats

Male Wistar rats weighing 300-350 g are anesthetized with thiopental (100 mg/kg i.p.). After tracheotomy, a catheter is introduced into the femoral artery to measure the blood pressure. The substances to be tested are administered orally in Transcutol, Cremophor EL, H₂O (10%/20%/70%) in a volume of 1 ml/kg.

All conventional modes of administration are suitable for administering the compounds of the formula (I), that is to say oral, parenteral, inhalation, nasal, buccal, sublingual, rectal or external such as, for example, transdermal, particularly preferably oral or parenteral. For parenteral administration, mention must be made in particular of intravenous, intramuscular and subcutaneous administration, for example as subcutaneous depot. Oral administration is very particularly preferred.

In this connection, the active substances can be administered alone or in the form of preparations. Preparations suitable for oral administration are, inter alia, tablets, capsules, pellets, coated tablets, pills, granules, solid and liquid aerosols, syrups, emulsions, suspensions and solutions. The active substance must be present therein in an amount such that a therapeutic effect is achieved. The active substance can be present in general in a concentration of from 0.1 to 100% by weight, in particular 0.5 to 90% by weight, preferably 5 to 80% by weight. The concentration of active substance ought in particular to be 0.5-90% by weight, i.e. the active substance should be present in amounts sufficient to achieve the indicated dosage range.

For this purpose, the active substances can be converted in a manner known per se into conventional preparations. This takes place by using inert, nontoxic, pharmaceutically suitable carriers, excipients, solvents, vehicles, emulsifers and/or dispersants.

Excipients which may be mentioned for example are: water, nontoxic organic solvents such as, for example, paraffins, vegetable oils (for example sesame oil), alcohols (for example ethanol, glycerol), glycols (for example polyethylene glycol), solid carriers such as natural or synthetic ground rocks (for example talc or silicates), sugars (for example lactose), emulsifiers, dispersants (for example polyvinylpyrrolidone) and lubricants (for example magnesium sulfate).

In the case of oral administration, tablets may of course also contain additions such as sodium citrate together with additives such as starch, gelatin and the like. Aqueous preparations for oral administration may furthermore be mixed with taste improvers or colors.

It has in general proved advantageous to administer amounts of about 0.1 to about 10 000 μg/kg, preferably about 1 to about 1 000 μg/kg, especially about 1 μg/kg to about 100 μg/kg of body weight on parenteral administration to achieve effective results. The amount on oral administration is about 0.1 to about 10 mg/kg, preferably about 0.5 to about 5 mg/kg of body weight.

It may nevertheless be necessary where appropriate to deviate from the amounts mentioned, specifically as a function of the body weight, administration route, individual behavior toward the active substance, mode of preparation and time or interval over which administration takes place.

Unless indicated otherwise, all percentage data are based on weight; ratios by volume are stated for mixtures of solvents.

B Preparation Examples

The following abbreviations are used in the examples:

A=the isomer eluted first (reversed phase)

B=the isomer eluted second (reversed phase)

DIEA=N,N-diisopropylethylamine

DMAP=4-dimethylaminopyridine

DMF=dimethylformamide

DMSO=dimethyl sulfoxide

EDCI=N′-(3-dimethylaminopropyl)-N-ethylcarbodiimide×HCl

EtOH=ethanol

HOBt=1-hydroxy-1H-benzotriazole×H₂O

HPLC=high pressure, high performance liquid chromatography

PyBOP®=benzotriazol-1-yloxytrispyrrolidinophosphonium hexafluorophosphate

rt=retention time

RT=room temperature

TBTU=O-(benzotriazol-1-yl)-N,N,N′,N′-tetramethyluronium tetrafluoroborate

TFA=trifluoroacetic acid

THF=tetrahydrofuran

Pd—C=palladium on carbon (10%)

HPLC Parameters:

Method 1: column: Kromasil C18; L-R temperature: 30° C.; flow rate=0.75 ml min⁻¹; eluent: A=0.01 M HClO₄, B═CH₃CN, gradient: 0.5 min 98% A, →4.5 min 10% A, →6.5 min 10% A.

Method 2: column: Kromasil C18 60*2; L-R temperature: 30° C.; flow rate=0.75 ml min⁻¹; eluent: A=0.01 M H₃PO₄, B═CH₃CN, gradient: →0.5 min 90% A, →4.5 min 10% A, →6.5 min 10% A.

Method 3: column: Kromasil C18 60*2; L-R temperature: 30° C.; flow rate=0.75 ml min⁻¹; eluent: A=0.005 M HClO₄, B═CH₃CN, gradient: →0.5 min 98% A, →4.5 min 10% A, →6.5 min 10% A.

Method 4: column: Symmetry C18, 2.1 mm×150 mm; column oven: 50° C.; flow rate=0.6 ml min⁻¹; eluent: A=0.6 g 30% HCl/l water, B═CH₃CN, gradient: 0.0 min 90% A, →4.0 min 10% A, →9 min 10% A.

Method 5: column: Kromasil 100 C18, 5 μm, 250 mm×20 mm, No. 1011312; temperature: 40° C.; flow rate=1.25 ml min⁻¹; eluent: 50% CH₃CN, 50% water.

Method 6: MHZ-2Q, Instrument Micromass Quattro LCZ

column: Symmetry C18, 50 mm×2.1 mm, 3.5 μm; temperature: 40° C.; flow rate=0.5 ml min⁻¹; eluent: A=CH₃CN+0.1% formic acid, eluent B=water+0.1% formic acid, gradient: 0.0 min 10% A, →4 min 90% A, →6 min 90% A.

Method 7: MHZ-2P, Instrument Micromass Platform LCZ

column: Symmetry C18, 50 mm×2.1 mm, 3.5 μm; temperature: 40° C.; flow rate=0.5 ml min⁻¹; eluent: A=CH₃CN+0.1% formic acid, eluent B=water+0.1% formic acid, gradient: 0.0 min 10% A, →4 min 90% A, →6 min 90% A.

Method 8: MHZ-7Q, Instrument Micromass Quattro LCZ

column: Symmetry C18, 50 mm×2.1 mm, 3.5 μm; temperature: 40° C.; flow rate=0.5 ml min⁻¹; eluent: A=CH₃CN+0.1% formic acid, eluent B=water+0.1% formic acid, gradient: 0.0 min 5% A, →1 min 5% A, →5 min 90% A →6 min 90% A.

Method 9: MHZ-1P, Instrument Micromass Platform LCZ

column: Symmetry C18, 150 mm×2.1 mm, 5 μm; temperature: 40° C.; flow rate=0.5 ml min⁻¹; eluent: A=CH₃CN+0.1% formic acid, eluent B=water+0.1% formic acid, gradient: 0.0 min 10% A, →9 min 90% A, →10 min 90% A.

Method 10: Instrument Finnigan MAT 900

column: Symmetry C18, 150 mm×2.1 mm, 3.5 μm; temperature: 40° C.; flow rate=0.9 ml min⁻¹; eluent: A=CH₃CN, eluent B=0.3 g 30% HCl/l water, gradient: 0.0 min 10% A, →3 min 90% A, →6 min 90% A.

Method 11: MHZ-2T, Instrument Micromass TOF-MUX Interface Quadruplicate Parallel Injection

column: Symmetry C18, 50 mm×2.1 mm, 3.5 μm; temperature: 24° C.; flow rate=0.75 ml min⁻¹; eluent: A=CH₃CN+0.1% formic acid, eluent B=water+0.1% formic acid, gradient: 0.0 min 10% A, →0.5 min 10% A, →4.0 min 90% A, →5.5 min 90% A.

Method 12:

column: Stability C30; 5 μm; 250* 3.0 mm; temperature: 25° C.; flow rate=0.60 ml min⁻¹; eluent: A=0.035 M HClO₄, B═CH₃CN, isocratic 55% A and 45% B (v/v)

Starting Compounds

### EXAMPLE I

### tert-Butyl (1S,2S)-2-(4-bromomethylphenyl)cyclohexane-1-carboxylate

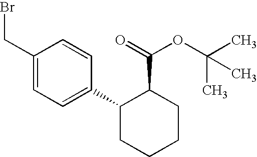

The intermediate is prepared in analogy to the method for the racemate (U.S. Pat. No. 5,395,840, column 17). For purification, the resulting mixture is triturated with diethyl ether or diisopropyl ether.

### EXAMPLE II

### tert-Butyl (1S,2S)-2-(4-formylphenyl)cyclohexanecarboxylate

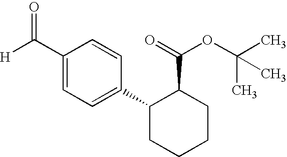

2 g of tert-butyl (1S,2S)-2-[4-(bromomethyl)phenyl]cyclohexanecarboxylate are stirred at room temperature with 1.3 g of N-methylmorpholine N-oxide and 2 g of molecular sieves (4 Å) in 40 ml of acetonitrile for 17 h. The mixture is filtered through kieselgur, and the solvent is removed in vacuo. The residue is taken up in dichloromethane, and the solution is extracted with 1N aqueous hydrochloric acid and saturated aqueous sodium chloride solution. The organic phase is dried and the solvent is removed in vacuo. 1.2 g of tert-butyl (1S,2S)-2-(4-formylphenyl)cyclohexanecarboxylate are obtained.

¹H NMR (300 MHz, CDCl₃): 1.14 (s, 9H), 1.45 (m, 4H), 1.84 (m, 4H), 2.51 (m, 1H), 2.81 (m, 1H), 7.38 (d, 2H), 7.80 (d, 2H), 9.97 (s, 1H).

### EXAMPLE III

### (1S,2S)-2-(4-Formylphenyl)cyclohexanecarboxylic acid

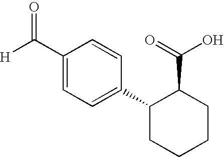

1.1 g of tert-butyl (1S,2S)-2-(4-formylphenyl)cyclohexanecarboxylate are dissolved in 20 ml of dichloromethane, and 2 ml of trifluoroacetic acid are added. The mixture is stirred at room temperature for 17 h and under reflux for 3 h. The solvent is removed in vacuo, and the residue is triturated with ether. 612 mg of (1S,2S)-2-(4-formylphenyl)cyclohexanecarboxylic acid are obtained (HPLC purity 81%), and are reacted without further purification.

¹H NMR (300 MHz, CDCl₃): 1.43 (m, 4H), 1.88 (m, 4H), 2.65 (m, 1H), 2.87 (m, 1H), 7.37 (d, 2H), 7.79 (d, 2H), 9.96 (s, 1H).

[A] General Method for Synthesizing Compounds of the Type:

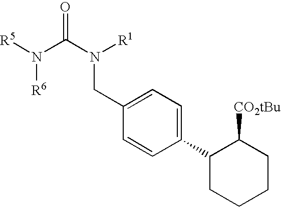

4.25 mmol of tert-butyl (1S,2S)-2-[4-(bromomethyl)phenyl]cyclohexane-carboxylate and 4.25 mmol of the appropriate urea are dissolved in 30 ml of dimethylformamide. 4.46 mmol of sodium hydride are added to this solution. The reaction is stirred at room temperature for 12 h. The solution is then poured into 600 ml of water and extracted with diethyl ether (three times with 50 ml each time). The combined extracts are washed with a saturated aqueous sodium chloride solution, dried over sodium sulfate, filtered and concentrated.

[B] General Method for Hydrolyzing the Tert-Butyl Ester:

200 mg of the appropriate tert-butyl ester derivative are introduced into 10 ml of a 4 M solution of hydrochloric acid in dioxane. The reaction is stirred at room temperature for 12 h and then concentrated. The residue is taken up in diethyl ether (10 ml each time) and evaporated to dryness five times.

[C] General Method for Amide Coupling:

[C-1]: 0.23 mmol of the appropriate carboxylic acid is dissolved in 5 ml of dimethylformamide under argon. 0.34 mmol of amine, 0.25 mmol of 1-hydroxybenzotriazole, 0.04 mmol of 4-dimethylaminopyridine and 0.26 mmol of N-(3-dimethylaminopropyl)-N′-ethylcarbodiimide hydrochloride are added. The solution is stirred at room temperature for 12 h. The mixture is purified directly by preparative HPLC.

[C-2]: The cyclohexanecarboxylic acid, PyBOP (1.5 eq.) and 4-dimethylaminopyridine (0.1 eq.) are dissolved in dimethylformamide (0.1 M) at room temperature, and N,N-diisopropylethylamine (2 eq.) is added. The reaction mixture is stirred for 15 min and, after addition of the amine (1.5 eq.), stirred at room temperature, where appropriate at 60° C., for 4-24 h. The desired product is purified by HPLC chromatography (acetonitrile/water mixtures).

[D] General Method for Synthesizing Tertiary Amines of the Type:

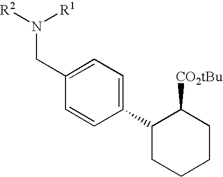

0.57 mmol of tert-butyl (1S,2S)-2-[4-(bromomethyl)phenyl]cyclohexane-carboxylate and 0.57 mmol of the appropriate secondary amine are dissolved in 5 ml of acetonitrile. 5.7 mmol of solid potassium carbonate are added to this, and the suspension is stirred at 60° C. for 4 h. The reaction solution is filtered and the filtrate is evaporated to dryness in a rotary evaporator.

The amines employed for reaction with cyclohexanecarboxylic acids are listed in the following table.

| Example | | |
|---|---|---|
| number | Amine | Preparation |
| IV | 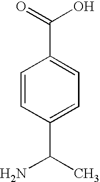 | from 4-acetylbenzoic acid as in Chem. Pharm. Bull. 1979, 27, 2735-2742 |
| V | 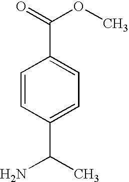 | reaction of 4-(1-aminoethyl)benzoic acid with TMS-diazomethane in methanol in analogy to J. Am. Chem. Soc. 1989, 111, 3062-3063 |
| VI | 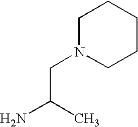 | by reaction of piperidine with 2-bromo-1- methylethylamine [Russ. J. Gen. Chem. 1994, 64, 1501 1502[ or nitroethane/formaldehyde [J. Chem. Soc. 1947, 1511] |
| VII | 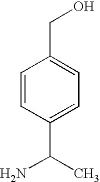 | by reduction of from 4-(1-aminoethyl)benzoic acid [Chem. Pharm. Bull. 1979, 27, 2735-2742] |
| VIII | 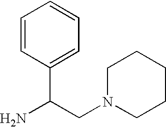 | from R-(−)-2-amino-2-phenylethanol in analogy to J. Med. Chem. 1994, 37, 913-923 |
| IX | 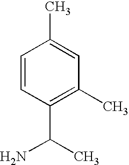 | from 1,4-dimethylacetophenone in analogy to J. Med. Chem. 1973, 16, 101-106 |
| X | 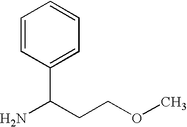 | from 3-amino-3-phenyl-1-propanol in analogy to Tetrahedron 2000,56, 3951-3961 |
| XI | 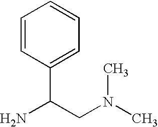 | from R-(−)-2-amino-2-phenylethanol in analogy to J. Med. Chem. 1999, 42, 1053-1065 |
| XII | 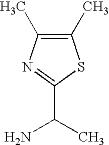 | from 4,5-dimethyithiazole [Bull. Soc. Chim. Fr. 1953, 702] in analogy to J. Chem. Soc. PT2 2000, 1339-1347 |
| XIII | 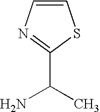 | from 2-acetyhhiazole in analogy to J. Chem. Soc. P12 2000, 1339-1347 |
| XIV | 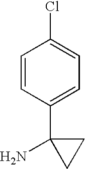 | from 1-(4-chlorophenyl)-1-cyclopropane- carboxylic acid as in Synth. Commun. 1980, 10, 107 |
| XV | 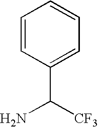 | from 2,2,2-trifluoro-1-phenylethanone as in J. Med. Chem. 1973, 16, 101-106 |
| XVI | 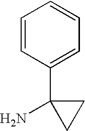 | from cyclopropyl phenyl ketone as in J. Med. Chem. 1973, 16, 101-106 |
| XVII | 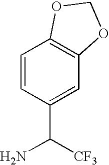 | from 3,4-(methylenedioxy)acetophenone as in J. Med. Chem. 1973, 16, 101-106 |
| XVIII | 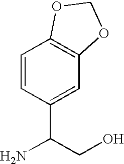 | from 3,4-(methylenedioxy)acetophenone via 1-(1,3-benzodioxol-5-yl)-2-bromoethylamine as in J. Med. Chem. 1996, 39, 253-266 |
| XIX | 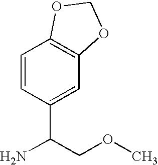 | from 2-amino-2-(1,3-benzodioxol-5-yl)ethanol as in Tetrahedron 2000,56, 3951-3961 |
| XX | 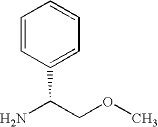 | from R-(−)-2-amino-2-phenylethanol as in Tetrahedron 2000,56, 3951-3961 |
| XXI | 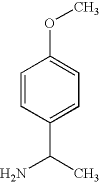 | from 4-methoxyacetophenone as in J. Chem. Soc. PT2 2000, 1339-1347 |
| XXII | 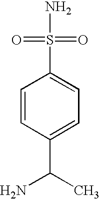 | from N-(phenylethyl)acetamide as in Hoppe-Seyler's Z. Physiol. Chem. 1944, 280, 35-37 |
| XXIII | 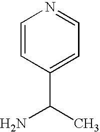 | Zh. Obshch. Khim. 1955, 25, 2453, 2456; Engl. ed. p. 2341 |
| XXIV | 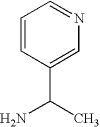 | J. Am. Chem. Soc. 1944, 66, 1293 |
| XXV | 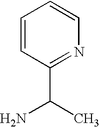 | J. Am. Chem. Soc. 1983, 105, 7075 |

### EXAMPLE XXVI

### (1S,2R)-2-[4-(Bromomethyl)phenyl]-N-[(1S)-1-phenylethyl]cyclohexane-carboxamide

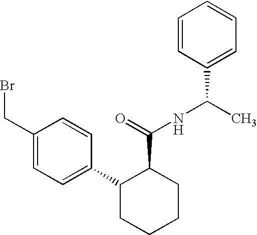

a) 15 g (42.46 mmol) of the compound from Example I are converted by general method [B] into the corresponding acid (yield: 13.2 g, quant.).

b) 2.9 g (24.23 mmol) of (S)-1-phenylethylamine are added at room temperature to a solution of 6 g (20.19 mmol) of (1S,2R)-2-[4-(bromomethyl)phenyl]cyclohexanecarboxylic acid, 2.9 g (24.23 mmol) of 1,3-dicyclohexylcarbodiimide and 0.3 g (2.5 mmol) of dimethylaminopyridine in 4.8 ml of dichloromethane, and the reaction mixture is stirred at room temperature for 14 h. For working up, the precipitate is filtered off, the solvent is removed in vacuo, and the crude product is purified by chromatography (silica gel 60, mobile phase: cyclohexane/ethyl acetate 8:2); yield: 3.97 g (49%) of the title compound.

R_{f}=0.33 (cyclohexane/ethyl acetate 7:3); ¹H NMR (200 MHz, DMSO): 1.1-1.9 (m, 10H), 2.4-2.9 (m, 2H), 4.6-4.8 (m, 3H), 6.6 (m, 2H), 6.9-7.4 (m, 7H), 8.0 (d, 1H).

### EXAMPLE XXVII

### (1S,2R)-2-[4-(Aminomethyl)phenyl]-N-[(1S)-1-phenylethyl]cyclohexanecarboxamide

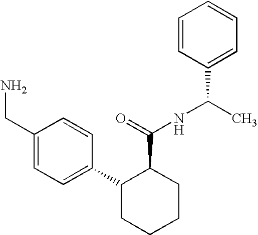

a) 770 mg (1.83 mmol) of (1S,2R)-2-[4-(bromomethyl)phenyl]-N-[(1S)-1-phenylethyl]cyclohexanecarboxamide (Example XXVI) and 131 mg (2.01 mmol) of sodium azide in 60 ml of dimethylformamide are stirred at 80° C. for 16 h. The reaction mixture is concentrated, mixed with ethyl acetate, washed with 50% saturated sodium chloride solution and dried over sodium sulfate. 600 mg (91%) of (1S,2R)-2-[4-(azidomethyl)phenyl]-N-[(1S)-1-phenylethyl]cyclohexanecarboxamide are obtained.

b) 600 mg (1.66 mol) of (1S,2R)-2-[4-(azidomethyl)phenyl]-N-[(1S)-1-phenylethyl]cyclohexanecarboxamide and catalyst (Pd/C, ca. 30 mg) are stirred at room temperature under hydrogen (1 atm) for 14 h. After filtration and removal of the solvent, the crude product is purified by HPLC, yield: 324 mg (58%) of the title compound.

¹H NMR (200 MHz, DMSO): 1.2 (d, 3H), 1.25-1.6 (m, 4H), 1.6-1.95 (m, 4H), 2.6 (m, 1H), 4.0 (bs, 2H), 4.65 (m, 1H), 6.6 (m, 2H), 7.0 (m, 3H), 7.15 (d, 2H), 7.25 (d, 2H), 7.95 (d, 1H).

### EXAMPLE XXVIII

### ((1S,2R)-2-{4-[(Cyclopropylamino)methyl]phenyl}-N-[(1S)-1-phenylethyl]cyclohexanecarboxamide

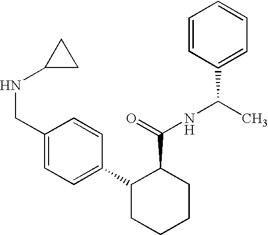

Prepared in analogy to general method [D] from 500 mg (1.25 mmol) of (1S,2R)-2-[4-(bromomethyl)phenyl]-N-[(1S)-1-phenylethyl]cyclohexanecarboxamide and 213.4 mg (3.75 mmol) of cyclopropylamine, yield: 342 mg (70%).

¹H NMR (200 MHz, CDCl₃): 0.3-0.7 (m, 4H), 1.25 (d, 3H), 1.3-1.6 (m, 3H), 1.7-2.1 (m, 4H), 2.15-2.25 (m, 2H), 2.75 (m, 1H), 3.7 (s, 2H), 4.85 (m, 1H), 5.2 (d, 1H), 6.65 (d, 2H), 7.1 (m, 7H).

### EXAMPLE XXIX

### (1S,2R)-2-(4-{[Ethyl(phenoxycarbonyl)amino]methyl}phenyl)cyclohexanecarboxylic acid

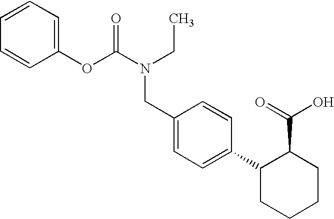

a) 0.69 ml (1.39 mmol) of a 2 M solution of ethylamine in methanol is added to 400 mg (1.39 mmol) of tert-butyl (1S,2S)-2-(4-formylphenyl)cyclohexanecarboxylate (Example II) and 0.05 ml (0.84 mmol) of acetic acid in 8 ml of methanol. The reaction mixture is stirred at room temperature for 3 h and, after addition of 105 mg (1.66 mmol) of sodium cyanoborohydride, stirred at room temperature for a further 18 h. The reaction mixture is concentrated and the crude product is employed directly for the next reaction.

b) 0.17 ml (0.136 mmol) of phenyl chloroformate and 0.24 ml (1.36 mmol) of N,N-diisopropylethylamine are added to 327 mg of tert-butyl (1S,2R)-2-{4-[(ethylamino)methyl]phenyl}cyclohexanecarboxylate (crude product) in 3 ml of dichloromethane, and the reaction mixture is stirred at room temperature for 14 h. For working up, the mixture is concentrated and the residue is purified by chromatography (silica gel 60, mobile phase: cyclohexane/ethyl acetate 8:2); yield: 327 mg (66%).

c) 264 mg (96%) of the title compound are prepared by general method [B] from 296 mg (0.68 mmol) of tert-butyl (1S,2R)-2-(4-{[ethyl(phenoxycarbonyl)amino]methyl}phenyl)cyclohexanecarboxylate.

¹H NMR (200 MHz, DMSO): 1.0-1.2 (m, 4H), 1.3-1.6 (m, 4H), 1.6-1.8 (m, 3H), 2.0 (d, 1H), 2.55 (m, 1H), 2.7 (m, 1H), 4.5 (m, 1H), 7.0-7.3 (m, 7H), 7.4 (m, 3H), 11.7 (bs, 1H).

The starting compounds listed in the following table are prepared in analogy to the method of Example XXIX.

| Example | | | HPLC method: |
|---|---|---|---|
| number | Structure | Mass | retention time |
| XXX | 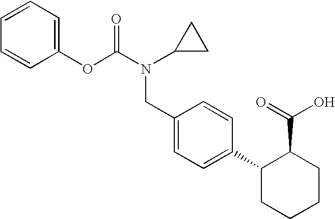 | MS (ESIpos): m/z = 394 (M + H)+ | Method 3: 4.93 min |
| XXXI | 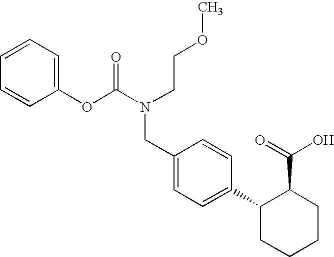 | MS (DCI): m/z = 429 M + NH4)+ | Method 3: 4.79 min |
| XXXII | 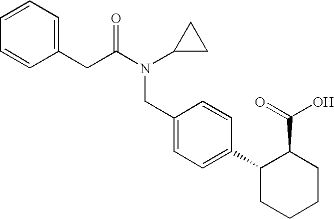 | MS (ESIpos): m/z = 392 (M + H)+ | Method 3: 4.8 min |
| XXXIII | 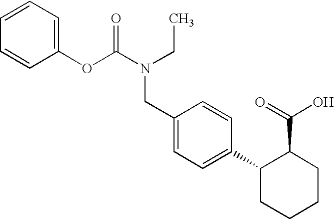 | MS (ESIpos): m/z = 382 (M + H)+ | Method 7: 4.6 min |
| XXXIV | 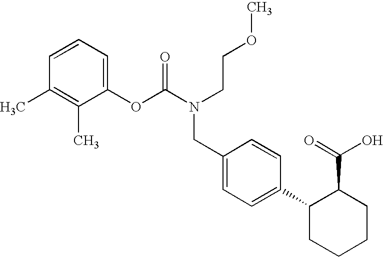 | MS (ESIpos): m/z = 439 (M + H)+ | Method 7: 4.7 min |
| XXXV | 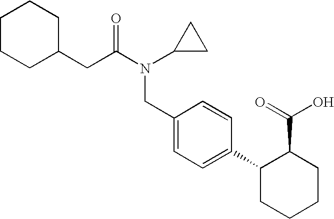 | MS (DCI): m/z = 415 (M + NH4)+ | Method 3: 5.11 min |
| XXXVI | 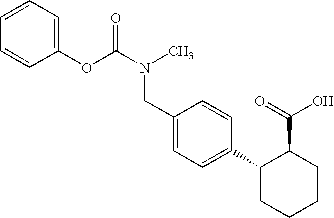 | MS (ESIpos): m/z = 385 (M + NH4)+ | Method 3: 4.84 min |
| XV | 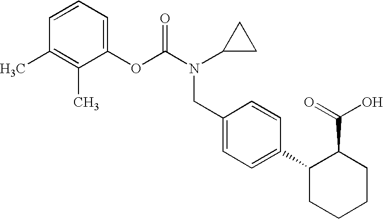 | MS (ESIpos): m/z = 439 (M + NH4)+ | Method 3: 4.91 min |

### EXAMPLE XXXVIII

### (1S,2R)-2-{4-[(Benzyloxy)carbonyl]phenyl}cyclohexanecarboxylic acid

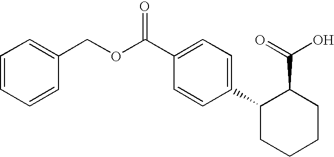

### a) 4-[(1R,2S)-2-(tert-Butoxycarbonyl)cyclohexyl]benzoic acid

A solution of potassium permanganate (42 g, 265.8 mmol) in water (1 l) is added to a solution of tert-butyl (1S,2R)-2-(4-formylphenyl)cyclohexanecarboxylate (35.0 g, 102.0 mmol) in acetone (500 ml) at room temperature. The reaction mixture is stirred at 40° C. for 2 h and, after addition of saturated aqueous sodium thiosulfate solution, the resulting suspension is filtered, the filter cake is washed with acetone, and the combined filtrates are concentrated in vacuo. The residue is taken up in ethyl acetate/water and, after phase separation, the aqueous phases are extracted with ethyl acetate. The combined organic phases are dried (sodium sulfate), filtered and concentrated in vacuo. The desired product is purified by flash chromatography (cyclohexane/ethyl acetate mixtures). Yield: 15 g (48%).

MS (DCI, NH₃): m/z=322 [M+NH₄]; HPLC (Method 3): rt=4.75 min.

### b) Benzyl 4-[(1R,2S)-2-(tert-butoxycarbonyl)cyclohexyl]benzoate

Benzyl bromide (4.1 ml, 34.5 mmol) is added dropwise to a solution of 4-[(1R,2S)-2-(tert-butoxycarbonyl)cyclohexyl]benzoic acid (7.00 g, 23.0 mmol) and cesium carbonate (8.24 g, 25.3 mmol) in acetonitrile (50 ml) at room temperature, and the mixture is stirred for 2 h. The reaction mixture is concentrated in vacuo, the residue is suspended in ethyl acetate, and the precipitate is filtered off and washed. The combined filtrates are concentrated in vacuo. The desired product is purified by flash chromatography (cyclohexane/ethyl acetate mixtures). Yield: 8.63 g (95%).

MS (DCI, NH₃): m/z=412 [M+NH₄]⁺; HPLC (Method 3): rt=6.13 min.

### c) (1S,2R)-2-{4-[(Benzyloxy)carbonyl]phenyl}cyclohexanecarboxylic acid

Trifluoroacetic acid (21.1 ml, 273 mmol) is added to a solution of benzyl 4-[(1R,2S)-2-(tert-butoxycarbonyl)cyclohexyl]benzoate (8.63 g, 21.8 mmol) in dichloromethane (140 ml) at room temperature. The reaction mixture is stirred at room temperature for 4 h, concentrated in vacuo, coevaporated with toluene three times, and dried. The crude product is employed without further purification in the next reaction. Yield: 7.5 g (100%).

MS (DCI, NH₃): m/z=356 [M+NH₄]⁺; HPLC (Method 3): rt=4.97 min.

[E] General Method for Reacting the (1S,2R)-2-{4-[(Benzyloxy)carbonyl]phenyl}cyclohexanecarboxanides to Give Benzoic Acid Derivatives

A solution of the (1S,2R)-2-{4-[(benzyloxy)carbonyl]phenyl}cyclohexanecarboxamide in tetrahydrofuran (0.1 M) is stirred in the presence of palladium (10% on carbon) under a hydrogen atmosphere for 6-20 h and, after filtration, the filtrate is concentrated in vacuo. The desired product can be purified by flash chromatography (cyclohexane/ethyl acetate mixtures).

The compounds listed in the following table are prepared by general method [E]:

| Example | | | HPLC method: |
|---|---|---|---|
| number | Structure | Mass | retention time |
| XXXIX | 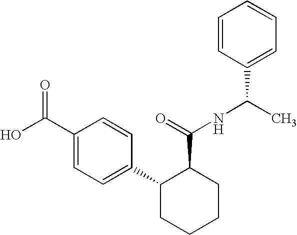 | MS (DCI, NH3): m/z =352 [M + H]+, 369 [M + NH4]+ | Method 3: 4.17 min |
| XL | 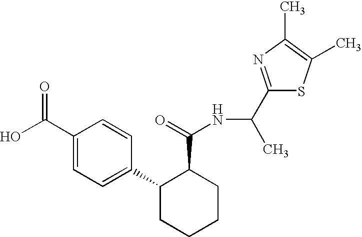 | MS (ESI): m/z =387 [M + H]+ | Method 6: 3.27 min 3.58 min |
| XLI | 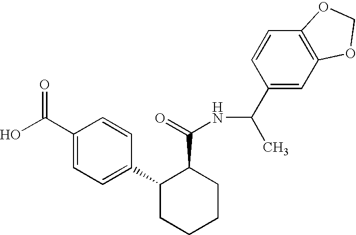 | MS (ESI): m/z =396 [M + H]+ | Method 10: 2.54 min 2.61 min |
| XLII | 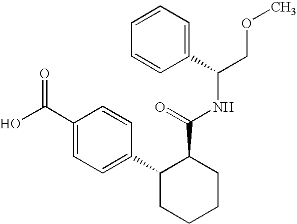 | MS (ESI): m/z =382 [M + H]+ | Method 7: 3.53 min |

[F] General Method for Reacting the Benzoic Acid Derivatives to Give Amides

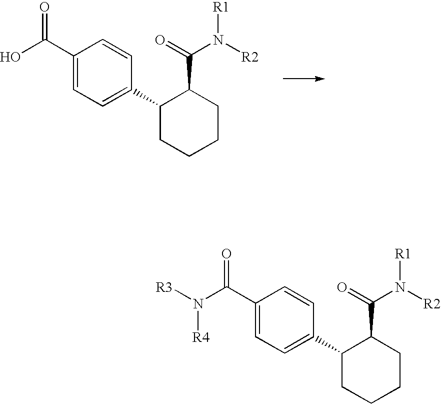

The cyclohexanecarboxylic acid, PyBOP (1.5 eq.) and 4-dimethylaminopyridine (0.1 eq.) are dissolved in dimethylformamide (0.1 M) at room temperature and N,N-diisopropylethylamine (2 eq.) is added. The reaction mixture is stirred for 15 min and, after addition of the amine (1.5 eq.), stirred at room temperature for 10-16 h. The desired product is purified by HPLC chromatography (acetonitrile/water mixtures).

The following table lists the amines employed for the reaction with benzoic acid derivatives:

| Example | | |
|---|---|---|
| number | Structure | Preparation |
| XLIII | 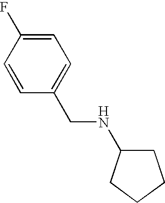 | by reductive amination as in J. Am. Chem. Soc. 1953, 75, 6258 or by nucleophilic substitution as in Tetrahedron 1995, 51, 7959-7980 |
| XLIV | 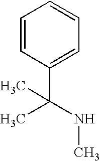 | as in Synthesis 1995, 1534-1538 |
| XLV | 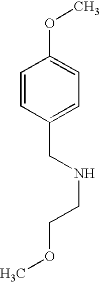 | from 4-methoxybenzylamine in analogy to Synthesis 1993, 1089-1091 |
| XLVI | 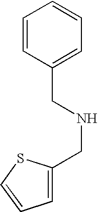 | by nucleophilic substitution as in J. Am. Chem. Soc. 1949, 71, 2905 |
| XLVII | 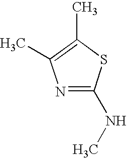 | as in Bull. Soc. Chim. Fr. 1954, 1048, J. Am. Chem. Soc. 1952, 74, 3868 |
| XLVIII | 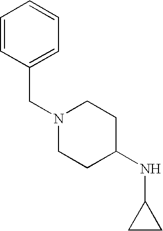 | in analogy to J. Med. Chem. 1996, 39, 3769-3789 |
| IL | 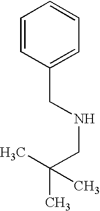 | as in Tetrahedron Lea. 1984, 25, 135-1638 or J. Chem. Soc. PT2 1994, 253-258 |

[H] General Method for Synthesizing Compounds of the Type:

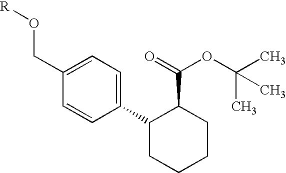

4.25 mmol of tert-butyl (1S,2S)-2-[4-(bromomethyl)phenyl]cyclohexane-carboxylate and 4.25 mmol of the appropriate alcohol are dissolved in dimethylformamide (30 ml). 4.46 mmol of sodium hydride are added to this solution at 0° C. The reaction is stirred at room temperature for 12 hours. The solution is then poured into 300 ml of water and extracted with diethyl ether (3 times with 50 ml). The combined extracts are washed with a saturated NaCl solution, dried with sodium sulfate, filtered and concentrated.

### Example L

### Methyl (1S*,2R*)-2-[3-(bromomethyl)phenyl]cyclohexanecarboxylate

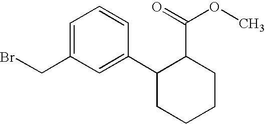

The intermediate is prepared in analogy to the synthesis of the starting compound of Example I from methyl m-methylcinnamate and butadiene (reaction in analogy to: U.S. Pat. No. 5,395,840 Example I and II) with subsequent bromination (in analogy to: U.S. Pat. No. 5,395,840 Example IV).

### Example LI

### (1S*,2S*)-2-(4-Hydroxyphenyl)-N-[(1S)-1-phenylethyl]cyclohexanecarboxamide

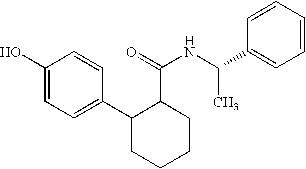

### a) Methyl (1S*,2S*)-2-(4-methoxyphenyl)cyclohexanecarboxylate

The intermediate is prepared in analogy to the synthesis of the starting compound of Example I from methyl ortho-methoxycinnamate and butadiene (reaction in analogy to: U.S. Pat. No. 5,395,840 Example I and II).

### b) (1S*,2S*)-2-(4-Methoxyphenyl)cyclohexanecarboxylic acid

A solution of methyl (1S*,2S*)-2-(4-methoxyphenyl)cyclohexanecarboxylate (1.0 g, 4.0 mmol) and lithium hydroxide (145 mg, 6.0 mmol) in methanol/water (3:1, 20 ml) is stirred at 60° C. for 2 d. After addition of water, methanol is removed in vacuo, and the aqueous residue is adjusted to pH 1 with 1 N hydrochloric acid. The precipitate which then separates out is filtered off, washed with water and dried in vacuo. Yield: 734 mg (78%) as mixture of diastereomers.

MS (ESI): m/z=217 [M+H—H₂O]⁺; HPLC (Method 7): rt=3.90 min.

### c) (1S*,2S*)-2-(4-Methoxyphenyl)-N-[(1S)-1-phenylethyl]cyclohexane-carboxamide

(1S*,2S*)-2-(4-Methoxyphenyl)cyclohexanecarboxylic acid (710 mg, 3.04mmol) is reacted with (S)-phenylethylamine (590 μl, 4.56 mmol) by general method [C-2]. The desired product is purified by flash chromatography (cyclohexane/dichloromethane mixtures). Yield: 852.1 mg (83%) as mixture of diastereomers.

MS (ESI): m/z=338 [M+H]⁺; HPLC (Method 3): rt=4.69 min, 4.75 min.

### d) (1S*,2S*)-2-(4-Hydroxyphenyl)-N-[(1S)-1-phenylethyl]cyclohexane-carboxamide

Boron tribromide (1 M in dichloromethane, 2 eq.) is added dropwise to a solution of (1S*,2S*)-2-(4-methoxyphenyl)-N-[(1S)-1-phenylethyl]cyclohexanecarboxamide (772 mg, 2.29 mmol) in dichloromethane (8 ml) at 0° C., and the mixture is stirred at room temperature for 20 h. The reaction mixture is put onto ice and neutralized with saturated aqueous sodium bicarbonate solution. The aqueous phase is acidified with 1 N hydrochloric acid and extracted with ethyl acetate. The combined organic phases are dried (sodium sulfate), filtered and concentrated in vacuo. The crude product is employed as the mixture of diastereomers without further purification in the next reaction.

MS (ESI): m/z=324 [M+H]⁺; HPLC (Method 6): rt=3.72 min, 3.83 min.

### SYNTHESIS EXAMPLES

### EXAMPLE 1

### N-[4-((1S,2S)-2-{[((1R)-2-Hydroxy-1-phenylethyl)amino]carbonyl}cyclohexyl)-benzyl]-N-phenyl-1-piperidinecarboxamide

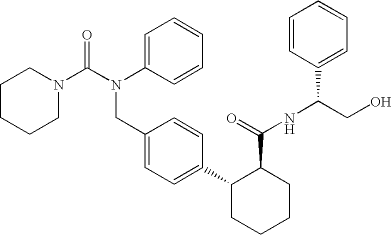

### a) tert-Butyl (1S,2S)-2-(4-{[phenyl(1-pyrrolidinylcarbonyl)amino]methyl}-phenyl)cyclohexanecarboxylate

The compound is synthesized by general method [A] starting from N-phenylpiperidylurea. The residue is purified by flash chromatography on silica gel (cyclohexane/ethyl acetate 10:1). 1.4 g. (68% yield) of the desired compound are obtained.

R_{f}=0.20 (petroleum ether/ethyl acetate 10:1); ¹H NMR (300 MHz, DMSO): 1.05 (s, 9H), 1.32 (m, 10H), 1.75 (m, 4H), 2.40 (m, 2H), 3.11 (m, 4H), 4.78 (s, 2H), 7.15 (m, 9H).

### b) (1S,2S)-2-(4-{[Phenyl(1-pyrrolidinylcarbonyl)amino]methyl}phenyl)-cyclohexanecarboxylic acid

The compound is synthesized -starting from the corresponding tert-butyl ester derivative by general hydrolysis method [B]. 156 mg (HPLC purity 90%) of the desired compound are obtained and reacted without further purification.

R_{f}=0.80 (dichloromethane/methanol 10:1); ¹H NMR (300 MHz, DMSO): 1.32 (m, 10H), 1.70 (m, 3H), 1.98 (m, 1H), 2.61 (m, 1H), 3.12 (m, 4H), 4.72 (s, 2H), 7.20 (m, 9H), 11.6 (bs, 1H).

### c) N-[4-((1S,2S)-2-{[((1R)-2-Hydroxy-1-phenylethyl)amino]carbonyl}-cyclohexyl)benzyl]-N-phenyl-1-piperidinecarboxamide

The compound is synthesized starting from (1S,2S)-2-(4-{[phenyl(1-pyrrolidinylcarbonyl)amino]methyl}phenyl)cyclohexanecarboxylic acid and (R)-phenylglycinol by general synthesis method [C] for amide coupling. 110 mg (yield 92%) of the desired compound are obtained.

R_{f}=0.17 (toluene/ethyl acetate 1:1); ¹H NMR (300 MHz, DMSO): 1.35 (m, 10H), 1.74 (m, 4H), 2.65 (m, 2H), 2.31 (m, 4H), 2.63 (m, 2H), 3.18 (m, 4H), 3.45 (m, 2H), 4.61 (m, 1H), 4.79 (m, 3H), 6.76 (m, 2H), 7.05 (m, 10H), 7.26 (m, 2H), 7.88 (d, 1H).

### EXAMPLE 2

### N-[4-((1R,2S)-2-{[((1R)-2-Hydroxy-1-phenylethyl)amino]carbonyl}cyclo-hexyl)benzyl]-N-phenyl-1-thiomorpholinecarboxamide

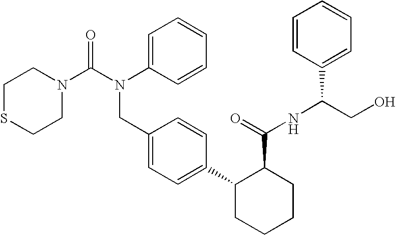

### a) tert-Butyl (1S,2S)-2-(4-{[phenyl(1-thiomorpholinylcarbonyl)amino]-methyl}phenyl)cyclohexanecarboxylate

The compound is synthesized by general method [A] starting from N-phenylthiomorpholinylurea. The residue is purified by flash chromatography on silica gel (cyclohexane/ethyl acetate 10:1). 1.6 g (72% yield) of the desired compound are obtained.

R_{f}=0.18 (petroleum ether/ethyl acetate 10:1); ¹H NMR (300 MHz, DMSO): 1.05 (s, 9H), 1.35 (m, 4H), 1.82 (m, 4H), 2.32 (m, 5H), 2.61 (s, 1H), 3.41 (m, 4H), 4.78 (s, 2H), 7.20 (m, 9H).

### b) (1S,2S)-2-(4-{[Phenyl(1-thiomorpholinylcarbonyl)amino]methyl}-phenyl)cyclohexanecarboxylic acid

The compound is synthesized starting from the corresponding tert-butyl ester derivative by general hydrolysis method [B]. 160 mg (HPLC purity 92%) of the desired compound are obtained and are reacted without further purification.

R_{f}=0.82 (dichloromethane/methanol 10:1); ¹H NMR (300 MHz, DMSO): 1.37 (m, 4H), 1.72 (m,.3H), 1.96 (m, 1H), 2.31 (m, 4H), 2.60 (m, 1H), 3.34 (m, 4H), 4.71 (s, 2H), 7.25 (m, 9H), 11.8 (bs, 1H).

### c) N-[4-((1R,2S)-2-{[((1R)-2-Hydroxy-1-phenylethyl)amino]carbonyl}-cyclohexyl)benzyl]-N-phenyl-1-thiomorpholinecarboxamide

The compound is synthesized starting from (1S,2S)-2-(4-{[phenyl(1-thiomorpholinylcarbonyl)amino]methyl}phenyl)cyclohexanecarboxylic acid and (R)-phenylglycinol by general synthesis method [C] for amide coupling. 105 mg (yield 94%) of the desired compound are obtained.

R_{f}=0.21 (toluene/ethyl acetate 1:1); ¹H NMR (300 MHz, DMSO): 1.47 (m, 11H), 2.32 (m, 4H), 2.61 (m, 2H), 3.45 (m, 6H), 4.59 (m, 1H), 4.79 (m, 3H), 6.71 (m, 2H), 7.05 (m, 10H), 7.30 (m, 2H), 7.88 (d, 1H).

### EXAMPLE 3

### N-[4-((1S,2S)-2-([((1R)-2-Hydroxy-1-phenylethyl)amino]carbonyl}cyclohexyl)-benzyl]-N-phenyl-1-N-methylpiperazinecarboxamide

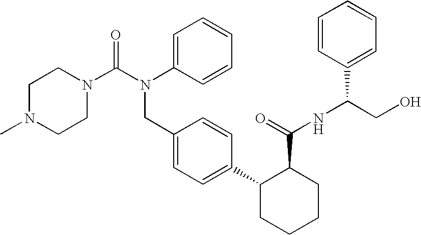

### a) tert-Butyl (1S,2S)-2-(4-{[phenyl(1-N-methylpiperazinylcarbonyl)-amino]methylphenyl)cyclohexanecarboxylate

The compound is synthesized by general method [A] starting from N-phenyl (N-methylpiperazyl)urea. The residue is purified by flash chromatography on silica gel (dichloromethane/methanol/triethylamine 10:1:0.1). 1.5 g (77% yield) of the desired compound are obtained.

R_{f}=0.14 (dichlormethane/methanol 10:1); ¹H NMR (300MHz, DMSO): 1.10 (s, 9H), 1.41 (m, 4H), 1.85 (m, 3H), 2.70 (m, 1H), 3.05 (s, 3H), 3.39 (m, 2H), 3.62 (m, 2H), 4.11 (d, 2H), 4.67 (s, 2H), 7.25 (m, 9H), 8.48 (s, 1H).

### b) (1S,2S)-2-(4-{[Phenyl(1-N-methylpiperazinylcarbonyl)amino]methyl}-phenyl)cyclohexanecarboxylic acid

The compound is synthesized starting from the corresponding tert-butyl ester derivative by general hydrolysis method [B]. 165 mg (HPLC purity 94%) of the desired compound are obtained and are reacted without further purification.

R_{f}=0.10 (dichloromethane/methanol 10: 1); ¹H NMR (300 MHz, DMSO): 1.39 (m, 4H), 1.78 (m, 3H), 1.90 (m, 1H), 2.78 (m, 1H), 3.05 (s, 3H), 3.39 (m, 2H), 3.62 (m, 2H), 4.12 (d, 2H), 4.61 (s, 2H), 7.25 (m, 9H), 8.91 (s, 1H).

### c) N-[4-((1S,2S)-2-{[((1R)-2-Hydroxy-1-phenylethyl)amino]carbonyl}-cyclohexyl)benzyl[-N-phenyl-1-N-methylpiperazinecarboxamide

The compound is synthesized starting from (1S,2S)-2-(4-{[phenyl(1-N-methylpiperazinylcarbonyl)amino]methyl}phenyl)cyclohexanecarboxylic acid and (R)-phenylglycinol by general synthesis method [C] for amide coupling. 98 mg (yield 84%) of the desired compound are obtained.

R_{f}=0.25 (dichloromethane/methanol 10:1); ¹H NMR (300 MHz, DMSO): 1.61 (m, 8H), 2.71 (m, 2H), 3.00 (s, 3H), 3.45 (m, 3H), 4.62 (m, 2H), 4.53 (m, 3H), 6.71 (m, 2H), 7.05 (m, 4H), 7.36 (m, 8H), 8.22 (d, 1H).

### EXAMPLE 4

### (1S,2S)-2-(4-{[Methyl(phenyl)amino]methyl}phenyl)-N-[(1S)-1-phenylethyl]-cyclohexanecarboxamide

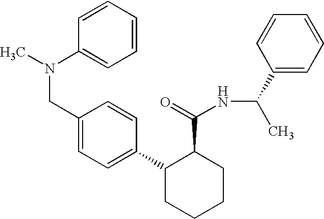

### a) tert-Butyl (1S,2S)-2-(4-{[methyl(phenyl)amino]methyl}phenyl)-cyclohexanecarboxylate

The compound is synthesized by general method [D] starting from N-methylaniline. 201 mg (HPLC purity 94%) of the desired compound are obtained and are reacted without further purification.

R_{f}=0.20 (cyclohexane/ethyl acetate (1:1); ¹H NMR (300 MHz, DMSO): 1.02 (s, 9H), 1.39 (m, 4H), 1.73 (m, 4H), 2.52 (m, 2H), 2.98 (s, 3H), 4.51 (s, 2H), 6.65 (m, 5H), 7.11 (m, 4H).

### b) (1S,2S)-2-(4-{[Methyl(phenyl)amino]methyl}phenyl)cyclohexane-carboxylic acid

The compound is synthesized starting from the corresponding tert-butyl ester derivative by general hydrolysis method [B]. 170 mg (HPLC purity 90%) of the desired compound are obtained and are reacted without further purification.

R_{f}=0.30 (dichloromethane/methanol 10:1); ¹H NMR (300 MHz, DMSO): 1.41 (m, 4H), 1.71 (m, 4H), 2.48 (m, 2H), 2.95 (s, 3H), 4.47 (s, 2H), 6.70 (m, 5H), 7.15 (m, 4H), 11.5 (bs, 1H).

### c) (1S,2S)-2-(4-{[Methyl(phenyl)amino]methyl}phenyl)-N-[(1S)-1-phenyl-ethyl]cyclohexanecarboxamide

The compound is synthesized starting from (1S,2S)-2-(4-{[methyl(phenyl)amino]methyl}phenyl)cyclohexanecarboxylic acid and (S)-phenylethylamine by general synthesis method [C] for amide coupling. 105 mg (yield 92%) of the desired compound are obtained.

R_{f}=0.51 (dichloromethane/methanol 10:1); ¹H NMR (300 MHz, DMSO): 0.85 (d, 3H), 1.35 (m, 4H), 1.71 (m, 4H), 2.28 (m, 1H), 2.65 (m, 2H), 2.99 (s, 3H), 4.50 (s, 2H), 4.61 (m, 1H), 6.55 (m, 1H), 6.70 (d, 2H), 7.15 (m, 11H), 7.80 (d, 1H).

### EXAMPLE 5

### (1S,2S)-2-(4-{[Allyl(phenyl)amino]methyl}phenyl)-N-[(1S)-1-phenylethyl]-cyclohexanecarboxamide

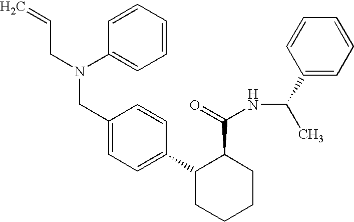

### a) tert-Butyl (1S,2S)-2-(4-{[allyl(phenyl)amino]methyl}phenyl)cyclo-hexanecarboxylate

The compound is synthesized by general method [D] starting from N-allylaniline. 220 mg (HPLC purity 96%) of the desired compound are obtained and are reacted without further purification.

R_{f}=0.17 (cyclohexane/ethyl acetate 1:1); ¹H NMR (300 MHz, DMSO): 1.10 (s, 9H), 1.45 (m, 4H), 1.88 (m, 4H), 2.41 (m, 1H), 2.65 (m, 1H), 3.99 (d, 2H), 4.50 (s, 2H), 5.18 (m, 2H), 5.91 (m, 1H), 6.70 (m, 4H), 7.11 (m, 5H).

### b) (1S,2S)-2-(4-{[Allyl(phenyl)amino]methyl}phenyl)cyclohexane-carboxylic acid

The compound is synthesized starting from the corresponding tert-butyl ester derivative by general hydrolysis method [B]. 180 mg (HPLC purity 92%) of the desired compound are obtained and are reacted without further purification.

R_{f}=0.25 (dichloromethane/methanol 10:1); ¹H NMR (300 MHz, DMSO): 1.46 (m, 4H), 1.78 (m, 4H), 2.40 (m, 1H), 2.47 (m, 1H), 4.00 (d, 2H), 4.55 (s, 2H), 5.20 (m, 2H), 5.98 (m, 1H), 6.82 (m, 4H), 7.18 (m, 5H), 11.2 (bs, 1H).

### c) (1S,2S)-2-(4-{[Allyl(phenyl)amino]methyl}phenyl)-N-[(1S)-1-phenyl-ethyl]cyclohexanecarboxamide

The compound is synthesized starting from (1S,2S)-2-(4-{[allyl(phenyl)amino]methyl})cyclohexanecarboxylic acid and (S)-phenylethyl-amine by general synthesis method [C] for amide coupling. 115 mg (yield 95%) of the desired compound are obtained.

R_{f}=0.53 (dichloromethane/methanol 10:1); ¹H NMR (300 MHz, DMSO): 0.87 (d, 3H), 1.37 (m, 4H), 1.69 (m, 4H), 2.25 (m, 1H), 2.65 (m, 2H), 3.99 (d, 2H), 4.51 (s, 2H), 4.62 (m, 1H), 5.18 (m, 2H), 5.88 (m, 1H), 6.56 (m, 1H), 6.70 (d, 2H), 7.16 (m, I1H), 7.83 (d, 1H).

The compounds listed in the following table are prepared in an analogous manner:

| Example | | | HPLC method: |
|---|---|---|---|
| number | Structure | Mass | retention time |
| 6 | 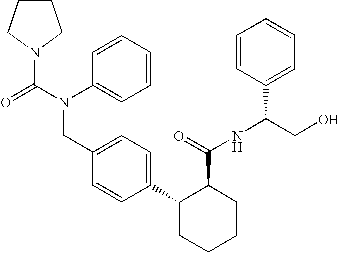 | MS (ESI): m/z (%) =526 (100) | Method 5: 2.87 min |
| 7 | 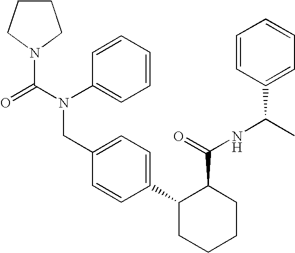 | MS (ESI): m/z (%) =510 (100) | Method 5: 3.17 min |
| 8 | 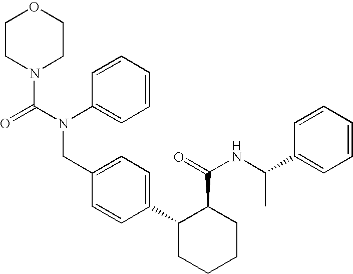 | MS (ESI): m/z (%) =526 (100) | Method 5: 2.95 min |
| 9 | 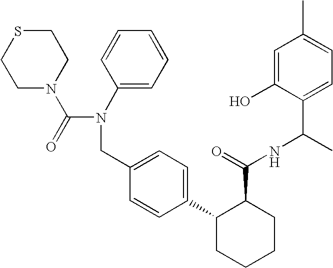 | MS (ESI): m/z (%) =571 (100) | Method 4: 4.73 min |
| 10 | 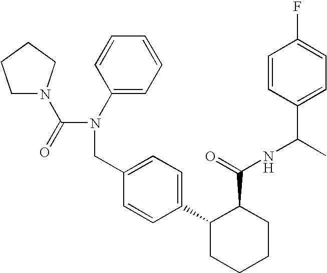 | MS (ESI): m/z (%) =527 (100) | Method 1: 4.88 min |
| 11 | 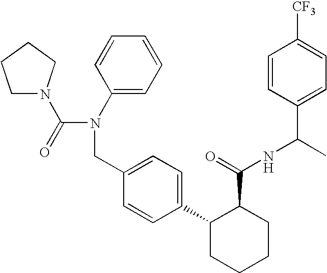 | MS (ESI): m/z (%) =577 (100) | Method 1: 5.06 min |
| 12 | 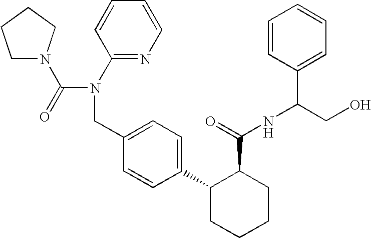 | MS (ESI): m/z (%) =526 (100) | Method 4: 3.64 min |
| 13 | 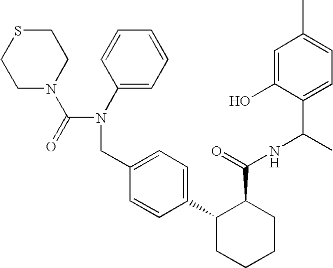 | MS (ESI): m/z (%) =571 (100) | Method 3: 6.63 min |
| 14 | 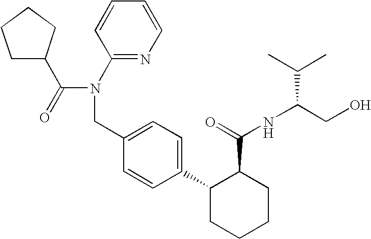 | MS (ESI): m/z (%) =492 (100) | Method 4: 3.47 min |
| 15 | 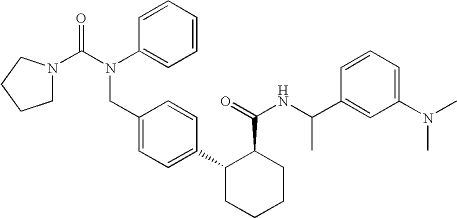 | MS (ESI): m/z (%) =553 (100) | Method 1: 4.43 min |
| 16 | 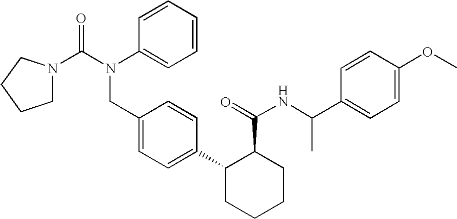 | MS (ESI): m/z (%) =540 (62), 406 (100) | Method 1: 4.89 min |
| 17 | 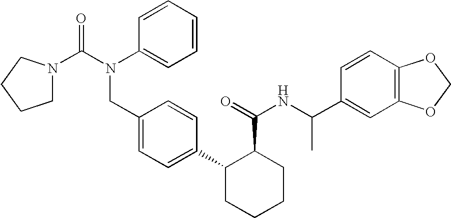 | MS (ESI): m/z (%) =554 (100) | Method 1: 4.86 min |
| 18 | 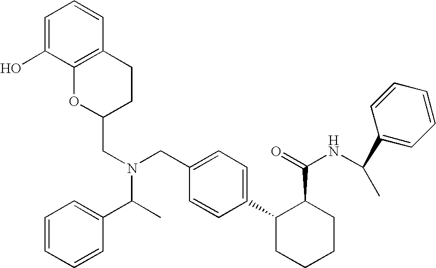 | MS (ESI): m/z (%) =616 (100) | Method 1: 4.84 min |
| 19 | 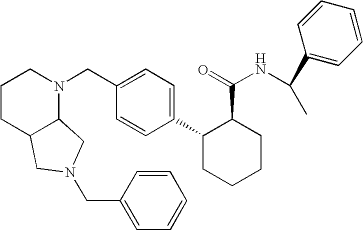 | MS (ESI): m/z (%) =535 (56), 581 (100) | Method 1: 3.47 min |
| 20 | 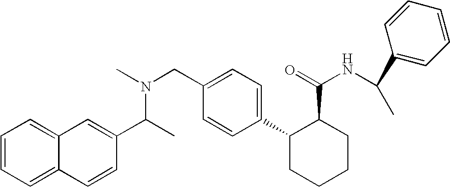 | MS (ESI): m/z (%) =504 (100) | Method 1: 3.65 min |
| 21 | 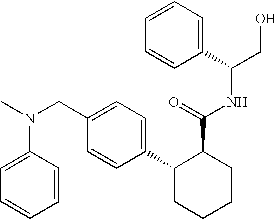 | MS (ESI): m/z (%) =442 (100) | Method 4: 4.20 min |
| 22 | 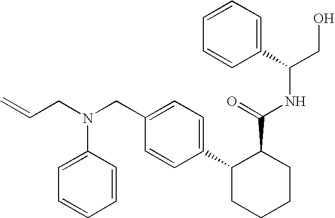 | MS (ESI): m/z (%) =468 (100) | Method 4: 4.63 min |
| 23 | 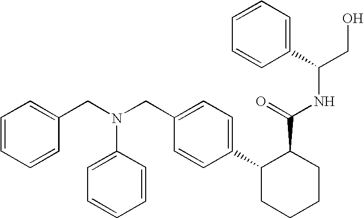 | MS (ESI): m/z (%) =518 (100) | Method 4: 4.87 min |
| 24 | 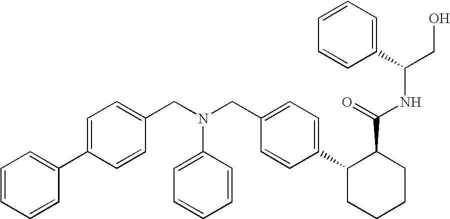 | MS (ESI): m/z (%) =594 (100) | Method 4: 5.32 min |
| 25 | 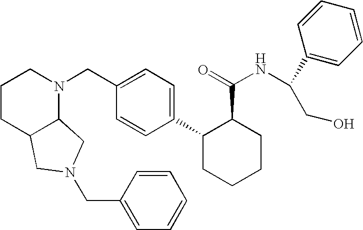 | MS (ESI): m/z (%) =551 (100) | Method 2: 1.53 min |
| 26 | 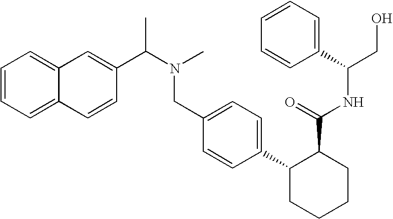 | MS (ESI): m/z (%) =520 (100) | Method 4: 2.93 min |
| 27 | 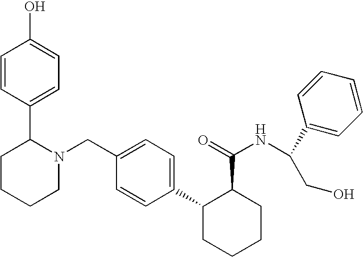 | MS (ESI): m/z (%) =526 (100) | Method 1: 3.69 min |
| 28 | 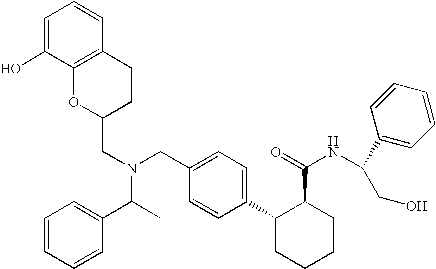 | MS (ESI): m/z (%) =632 (100) | Method 4: 3.24 min |
| 114 | 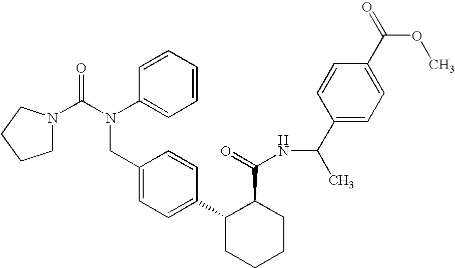 | MS (ESI): m/z = 568 [M + H]+ | Method 3: 4.88 min, 4.95 min |
| 115 | 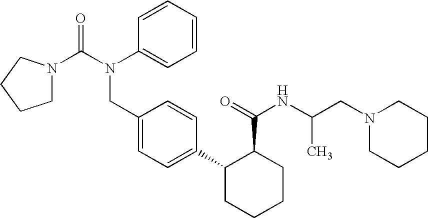 | MS (ESI): m/z = 531 [M + H]+ | Method 6: 3.10 min |
| 116 | 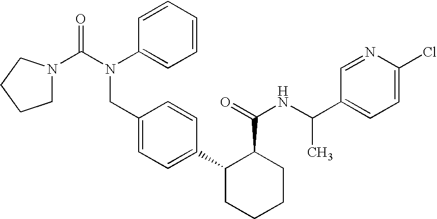 | MS (ESI): m/z = 545 [M + H]+ | Method 13: 4.40 min |
| 117 | 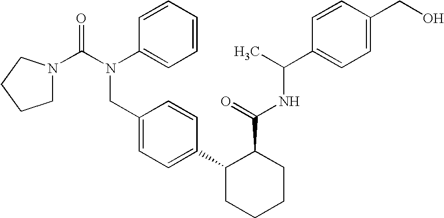 | MS (ESI): m/z = 540 [M + H]+ | Method 13: 4.12 min |
| 118 | 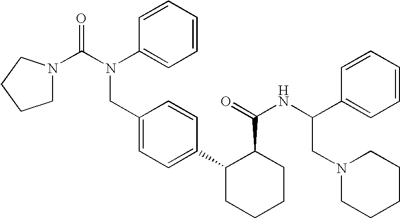 | MS (ESI): m/z = 593 [M + H]+ | Method 3: 4.60 min |
| 119 | 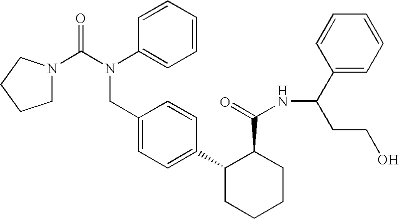 | MS (ESI): m/z = 540 [M + H]+ | Method 3: 4.61 min |
| 120 | 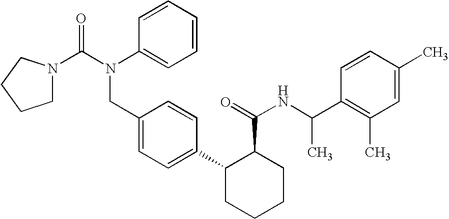 | MS (DCI, NH3): m/z = 538 [M + H]+, 555 [M + NH4]+ | Method 3: 5.18 min, 5.25 min |
| 121 | 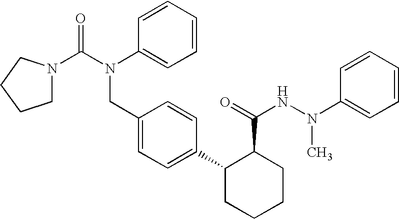 | MS (ESI): m/z = 511 [M + H]+ | Method 7: 4.53 min |
| 122 | 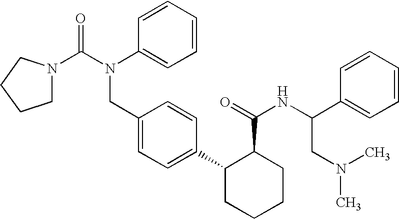 | MS (ESI): m/z = 553 [M + H]+ | Method 3: 4.52 min |

### EXAMPLE 123

### (1S,2S)-2-(4-{[Dimethylbenzyl(methyl)amino]methyl}phenyl)-N-[(1S)-1-phenylethyl]cyclohexanecarboxamide

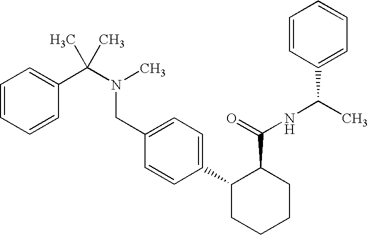

### a) tert-Butyl (1S,2S)-2-(4-{[dimethylbenzyl(methyl)amino]methyl}-phenyl)cyclohexanecarboxylate

The compound is synthesized by general method [D] starting from N-dimethylbenzyl-N-methylamine. 280 mg (HPLC purity 98%) of the desired compound are obtained and are reacted without further purification.

R_{f}=0.22 (cyclohexane/ethyl acetate 1:1); ¹H NMR (300 MHz, DMSO): 1.10 (s, 9H), 1.44 (m, 4H), 1.49 (s, 6H), 1.88 (m, 4H), 2.05 (s, 3H), 2.40 (m, 1H), 2.62 (m, 1H), 4.50 (s, 2H), 6.70 (m, 4H), 7.11 (m, 5H).

### b) (1S,2S)-2-(4-{[Dimethylbenzyl(methyl)amino]methyl}phenyl)cyclo-hexanecarboxylic acid

The compound is synthesized starting from the corresponding tert-butyl ester derivative by general hydrolysis method [B]. 210 mg (HPLC purity 95%) of the desired compound are obtained and are reacted without further purification.

R_{f}=0.31 (dichloromethane/methanol 10:1); ¹H NMR (300 MHz, DMSO): 1.46 (m, 4H), 1.48 (s, 6H), 1.78 (m, 4H), 2.05 (s, 3H), 2.53 (m, 2H), 3.47 (s, 2H), 6.82 (m, 4H), 7.18 (m, 5H), 11.6 (bs, 1H).

### c) (1S,2S)-2-(4-{[Dimethylbenzyl(methyl)amino]methyl}phenyl)-N-[(1S)-1-phenylethyl]cyclohexanecarboxamide

The compound is synthesized starting from (1S,2S)-2-(4-{[dimethylbenzyl(methyl)amino]methyl})cyclohexanecarboxylic acid and (S)-phenylethylamine by general synthesis method [C] for amide coupling. 160 mg (yield 93%) of the desired compound are obtained.

R_{f}=0.60 (dichloromethane/methanol 10:1); ¹H NMR (300 MHz, DMSO): 1.10 (d, 3H), 1.37 (m, 4H), 1.49 (s, 6H), 1.72 (m, 4H), 2.05 (s, 3H), 2.65 (m, 2H), 3.32 (s, 2H), 4.67 (m, 1H), 6.56 (m, 1H), 7.16 (m, 13H), 7.91 (d, 1H).

### EXAMPLE 124

### (1S,2S)-2-(4-{[Dimethylbenzyl(methyl)ammoniumhydrochloride]methyl}phenyl)-N-[(1S)-1-phenylethyl]cyclohexanecarboxamide

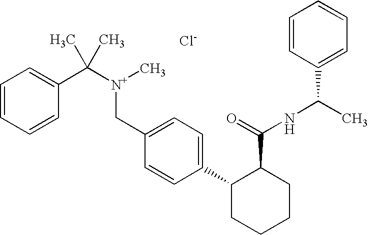

200 mg of (1S,2S)-2-(4-{[dimethylbenzyl(methyl)amino]methyl}phenyl)-N-[(1S)-1-phenylethyl]cyclohexanecarboxamide are dissolved in 5 ml of 4 M solution of HCL in dioxane and lyophilized. 210 mg (yield 100%) of the desired product are obtained as a colorless solid.

R_{f}=0.25 (dichloromethane/methanol 10:1); ¹H NMR (300 MHz, DMSO): 1.11 (d, 3H), 1.37 (m, 4H), 1.53 (s, 6H), 1.72 (m, 4H), 2.20 (s, 3H), 2.65 (m, 2H), 3.32 (s, 2H), 4.67 (m, 1H), 6.56 (m, 1H), 7.16 (m, 13H), 7.91 (d, 1H), 11.5 (s, 1H).

### EXAMPLE 125

### (1S,2S)-2-(4-{[Dimethylbenzylamino]methyl}phenyl)-N-[(1S)-1-phenylethyl]-cyclohexanecarboxamide

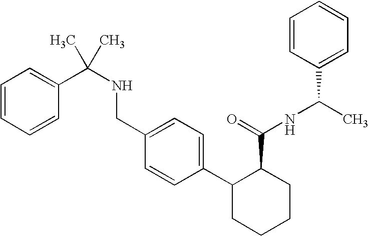

### a) tert-Butyl (1S,2S)-2-(4-{[dimethylbenzylamiino]methyl}phenyl)cyclo-hexanecarboxylate

The compound is synthesized by general method [D] starting from N-dimethylbenzylamine. 280 mg (HPLC purity 96%) of the desired compound are obtained and are reacted without further purification.

R_{f}=0.18 (cyclohexane/ethyl acetate 1:1) ¹H NMR (300 MHz, DMSO): 1.10 (s, 9H), 1.44 (m, 4H), 1.49 (s, 6H), 1.88 (m, 4H), 2.40 (m, 1H), 2.60 (s, 1H), 2.62 (m, 1H), 4.50 (s, 2H), 6.70 (m, 4H), 7.11 (m, 5H).

### b) (1S,2S)-2-(4-{[Dimethylbenzyl(methyl)amino]methyl}phenyl)cyclo-hexanecarboxylic acid

The compound is synthesized starting from the corresponding tert-butyl ester derivative by general hydrolysis method [B]. 210 mg (HPLC purity 95%) of the desired compound are obtained and are reacted without further purification.

R_{f}=0.23 (dichloromethane/methanol 10:1) ¹H NMR (300 MHz, DMSO): 1.46 (m, 4H), 1.48 (s, 6H), 1.78 (m, 4H), 2.53 (m, 2H), 2.60 (s, 1H), 3.47 (s, 2H), 6.82 (m, 4H), 7.18 (m, 5H), 11.6 (bs, 1H).

### c) (1S,2S)-2-(4-{[Dimethylbenzylamino]methyl}phenyl)-N-[(1S)-1-phenyl-ethyl]cyclohexanecarboxamide

The compound is synthesized starting from (1S,2S)-2-(4-{[dimethylbenzylamino]methyl))cyclohexanecarboxylic acid and (S)-phenyl-ethylamine by general synthesis method [C] for amide coupling. 140 mg (yield 89%) of the desired compound are obtained.

R_{f}=0.60 (dichloromethane/methanol 10:1) ¹H NMR (300 MHz, DMSO): 1.13 (d, 3H), 1.37 (m, 4H), 1.49 (s, 6H), 1.72 (m, 4H), 2.40 (s, 1H), 2.65 (m, 2H), 3.32 (s, 2H), 4.67 (m, 1H), 6.56 (m, 1H), 7.16 (m, 13H), 7.90 (d, 1H).

### EXAMPLE 126

### (1S,2S)-2-(4-{[Dimethylbenzylamino]methyl}phenyl)-N-methyl-N-[(1S)-1-phenylethyl]cyclohexanecarboxamide

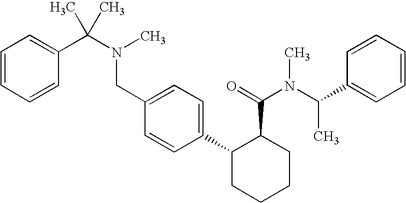

The compound is synthesized starting from (1S,2S)-2-(4-{[dimethylbenzylamino]methyl))cyclohexanecarboxylic acid and (S)-N-methyl-N-phenylethylamine by general synthesis method [C] for amide coupling. 89 mg (yield 62%) of the desired compound are obtained.

R_{f}=0.88 (dichloromethane/methanol 10:1) ¹H NMR (300 MHz, DMSO): 1.13 (d, 3H), 1.37 (m, 4H), 1.49 (s, 6H), 1.72 (m, 4H), 2.05 (s, 3H), 2.40 (s, 1H), 2.65 (m, 2H), 3.32 (s, 2H), 3.50 (s, 3H), 4.67 (m, 1H), 6.56 (m, 1H), 7.16 (m, 13H), 7.80 (d, 1H).

The compounds listed in the following table are prepared in an analogous manner:

| Example | | | HPLC method: |
|---|---|---|---|
| number | Structure | Mass | retention time |
| 127 | 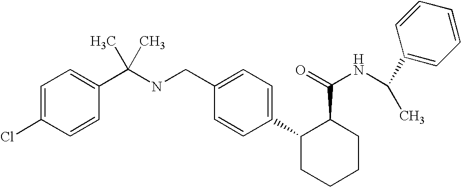 | MS (ESI): m/z (%) =490 (100) | Method 1: 3.17 min |
| 128 | 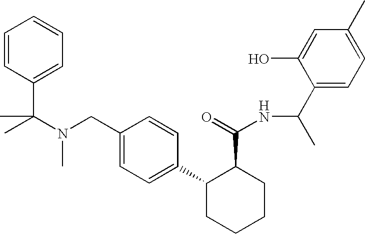 | MS (ESI): m/z = 500 [M + H]⁺ | Method 3: 3.45 min |
| 129 | 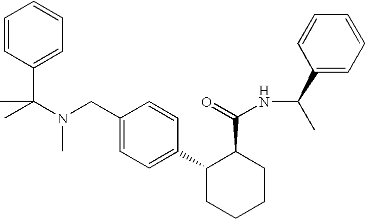 | MS (ESI): m/z = 470 [M + H]⁺ | Method 3: 4.38 min |
| 130 | 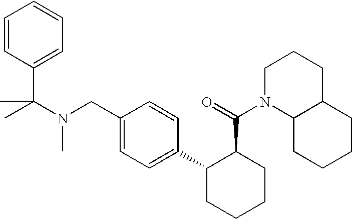 | MS (ESI): m/z = 488 [M + H]⁺ | Method 3: 3.26 min, 3.30 min |
| 131 | 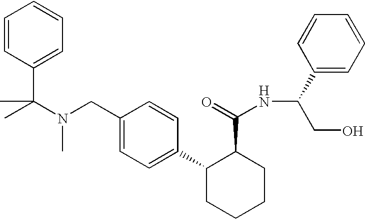 | MS (ESI): m/z = 486 [M + H]⁺ | Method 3: 2.63 min |
| 132 | 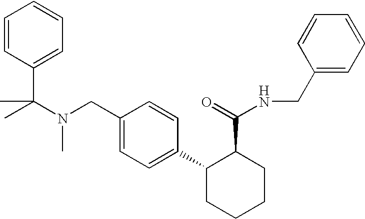 | MS (ESI): m/z = 456 [M + H]⁺ | Method 3: 2.97 min |
| 133 | 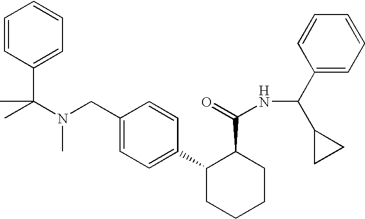 | MS (ESI): m/z = 496 [M + H]⁺ | Method 3: 3.18 min, 3.20 min |
| 134 | 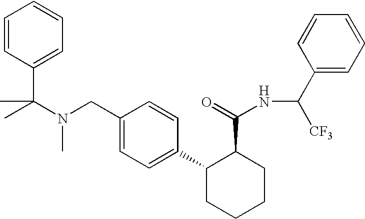 | MS (ESI): m/z = 524 [M + H]⁺ | Method 3: 3.29 min, 3.32 min |
| 135 | 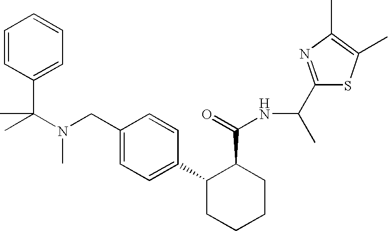 | MS (ESI): m/z = 504 [M + H]⁺ | Method 10: 1.88 min, 2.00 min |
| 136 | 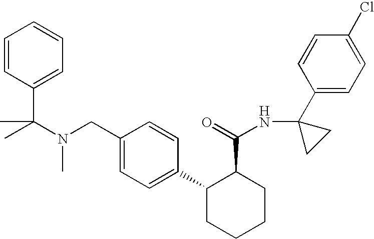 | MS (ESI): m/z = 560 [M + H]⁺ | Method 10: 2.14 min |
| 137 | 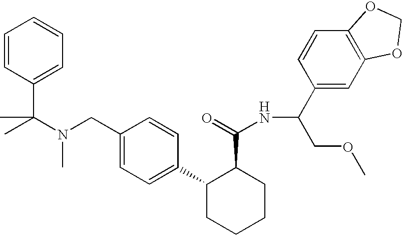 | MS (ESI): m/z = 543 [M + H]⁺ | Method 10: 1.94 min |
| 138 | 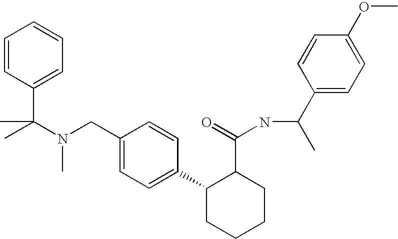 | MS (ESI): m/z = 499 [M + H]⁺ | Method 3: 4.29 min, 4.34 min |
| 139 | 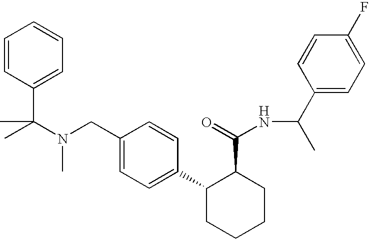 | MS (ESI): m/z = 487 [M + H]⁺ | Method 10: 1.93 min, 2.12 min |
| 140 | 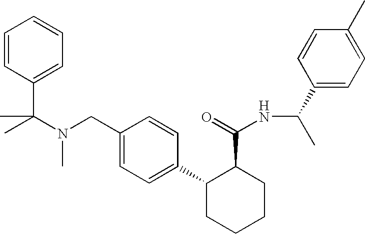 | MS (ESI): m/z = 483 [M + H]⁺ | Method 3: 4.52 min |
| 141 | 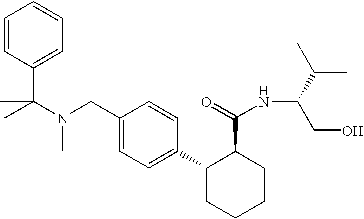 | MS (ESI): m/z = 451 [M + H]⁺ | Method 3: 4.01 min |
| 142 | 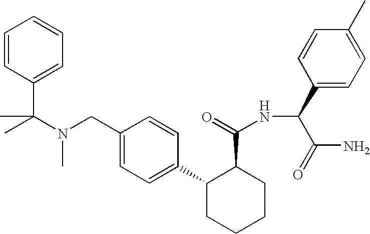 | MS (ESI): m/z = 498 [M + H]⁺ | Method 3: 4.04 min |
| 143 | 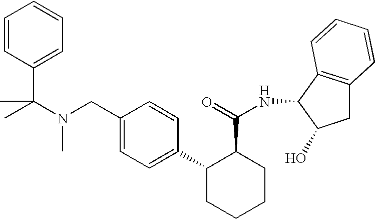 | MS (DCI, NH₃): m/z = 497 [M + H]⁺ | Method 3: 4.20 min |
| 144 | 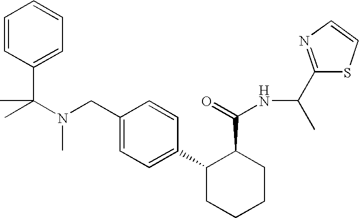 | MS (ESI): m/z = 476 [M + H]⁺ | Method 3: 4.02 min, 4.10 min |

### EXAMPLE 145

### 3,4-Dimethyl-N-{4-[(1R,2S)-2-({[(1S)-1-phenylethyl]amino}carbonyl)cyclo-hexyl]benzyl}benzamide

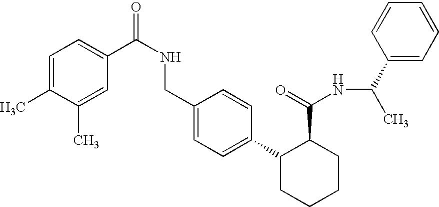

0.02 ml (0.11 mmol) of N,N-diisopropylethylamine is added to 37 mg (0.1 mmol) of (1S,2R)-2-{4-[(aminomethyl]phenyl}-N-[(1S)-1-phenylethyl]cyclohexane-carboxamide (Example XXVII) and 18.5 mg (0.11 mmol) of 3,4-dimethylbenzoyl chloride in 1 ml of dichloromethane. The reaction mixture is stirred at room temperature for 1 h and directly purified by HPLC, yield 38 mg (81%).

R_{f}=0.42 (cyclohexane/ethyl acetate 1:1); MS (DCI): m/z=486 (M+NH₄)⁺; ¹H NMR (200 MHz, DMSO): 1.2 (d, 3H), 1.35-1.9 (m, 8H), 2.25 (s, 6H), 4.4 (d, 2H), 4.7 (m, 1H), 6.6 (d, 2H), 7 (m, 8H), 7.6 (d, 1H), 7.7 (s, 1H), 8.0 d, 1H), 8.85 (t, 1H).

The compounds listed in the following table are prepared in analogy to the method of Example 145.

| Example | | | HPLC method: |
|---|---|---|---|
| number | Structure | Mass | retention time |
| 146 | 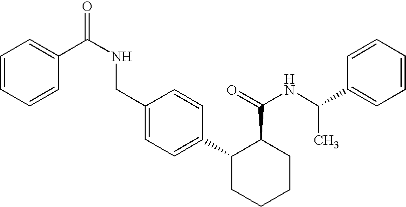 | MS (DCI): 458 (M + NH₄)⁺ | Method 3: 4.52 min |
| 147 | 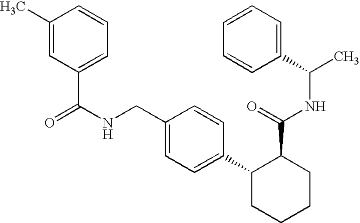 | MS (ESIpos): 455 (M + H)⁺ | Method 3: 4.61 min |
| 148 | 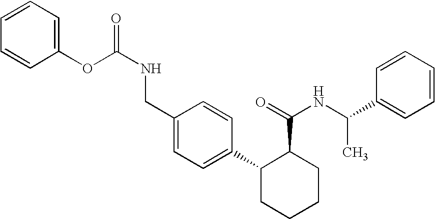 | MS (ESIpos): 513 (M + H)⁺ | Method 3: 4.70 min |
| 149 | 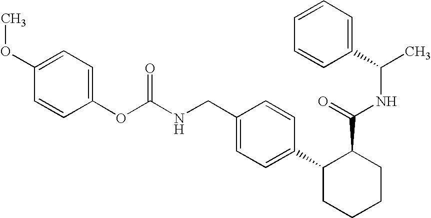 | MS (ESIpos): 487 (M + H)⁺ | Method 3: 4.69 min |
| 150 | 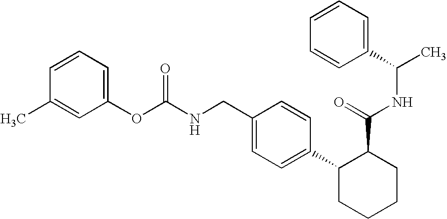 | MS (ESIpos): 471 (M + H)⁺ | Method 3: 4.86 min |
| 151 | 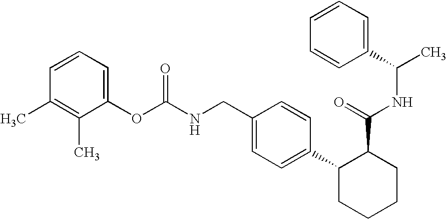 | MS (ESIpos): 507 (M + Na)⁺ | Method 3: 5.03 min |
| 152 | 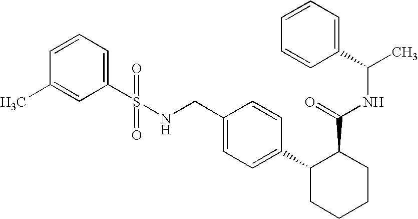 | MS (DCI): 491 (M + NH₄)⁺ | Method 3: 4.72 min |
| 153 | 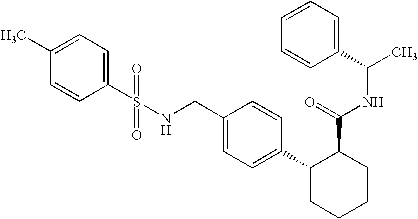 | MS (DCI): 491 (M + NH₄)⁺ | Method 3: 4.72 |

### EXAMPLE 154

### (1S,2R)-2-(4-{[Cyclopropyl(phenoxyacetyl)amino]methyl}phenyl)-N-[(1S)-1-phenylethyl]cyclohexanecarboxamide

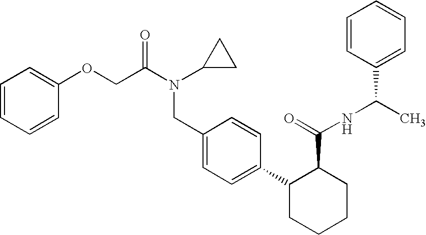

30 mg (0.15 mmol) of bromoacetyl bromide and 27.5 mg (0.21 mmol) of N,N-diisopropylethylamine are added to a solution of 40 mg (0.11 mmol) of (1S,2R)-2-{4-[(cyclopropylamino)methyl]phenyl }-N-[(1S)-1-phenylethyl]cyclohexane-carboxamide (Example XXVIII) in 2 ml of THF. The reaction mixture is stirred at room temperature for 1 h and added to a suspension of 40 mg (0.42 mmol) of phenol and 146.8 mg (1.06 mmol) of potassium carbonate in 2 ml of THF. After 14 h at room temperature, the solvent is removed and the residue is purified by HPLC. Yield: 41 mg (76%).

R_{f}=0.3 (cyclohexane/ethyl acetate 1:1); MS (ESIpos): m/z=511 (M+H)⁺; ¹H NMR (200 MHz, DMSO): 0.7-0.9 (m, 4H), 1.2 (d, 3H), 1.35-1.6 (m, 4H), 1.7-1.95 (m, 4H), 2.5-2.7 (m, 2H), 4.45 (d, 1H), 4.6 (d, 1H), 4.7 (m, 1H), 5.05 (s, 2H), 6.65 (m, 2H), 6.9-7.15 (m, 10H), 7.3 (t, 2H), 7.95 (d, 1H).

The compounds listed in the following table are prepared in analogy to the method of Example 154.

| Example | | | HPLC method: |
|---|---|---|---|
| number | Structure | Mass | retention time |
| 155 | 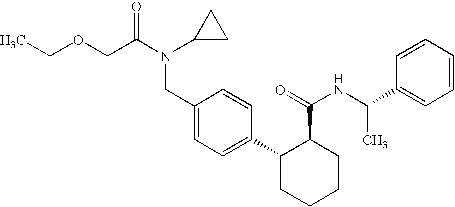 | MS (ESIpos): 463 (M + H)⁺ | Method 7: 3.97 min |
| 156 | 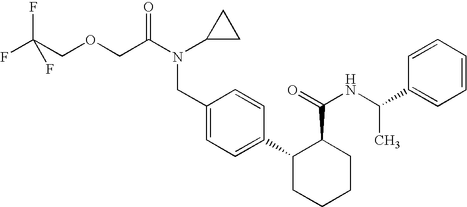 | MS (ESIpos): 517 (M + H)⁺ | Method 3: 4.77 min |

### EXAMPLE157

### 5-(Methylsulfanyl)-1,2,4-thiadiazol-3-yl cyclopropyl{4-[(1R,2S)-2-({[(1S)-1-phenylethyl]amino}carbonyl)cyclohexyl]benzyl}carbamate

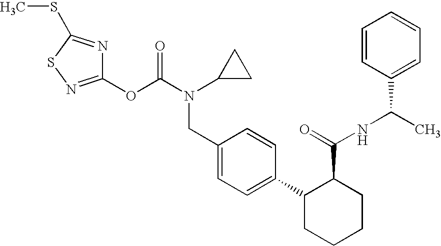

a) A solution of 102 mg (0.34 mmol) of bis(trichloromethyl) carbonate (triphosgene) in 1.5 ml of dichloromethane is added dropwise to a solution of 300 mg (0.80 mmol) of (1S,2R)-2-{4-[(cyclopropylamino)methyl]phenyl}-N-[(1S)-1-phenylethyl]cyclohexanecarboxamide (Example XXVIII) and 120 mg (1.2 mmol) of triethylamine in 3 ml of dichloromethane at 0° C. The reaction mixture is stirred at room temperature for a further 3 h, the resulting solution is washed with cold 10% hydrochloric acid solution, cold saturated sodium bicarbonate solution and cold saturated sodium chloride solution and dried over sodium sulfate, and the solvent is removed. Yield 270 mg.

b) 90 mg (0.17 mmol) of the crude product in 1 ml of acetonitrile are added at 82° C. to a suspension of 44 mg (0.3 mmol) of 5-methylthio[1.2.4]thiadiazol-3-ole [J. Heterocycl. Chem. 1979, 16, 961-971) and 370 mg of poly(4-vinylpyridine) and 3 ml of acetonitrile. The reaction mixture is heated under reflux for a further 14 h. The suspension is filtered, the solvent is removed, and the residue is purified by HPLC, yield: 55 mg (33%) of the title compound.

R_{f}=0.13 (cyclohexane/ethyl acetate 1:1); MS (ESIpos): n/z =551 (M+H)⁺; ¹H NMR (200 MHz, DMSO): 0.7-0.9 (m, 4H), 1.2 (d, 3H), 1.3-1.6 (m, 4H), 1.7-1.9 (m, 4H), 2.5-2.7 (m, 6H), 4.4-4.6 (m, 2H), 4.7 (m, 1H), 6.65 (m, 2H), 6.9-7.2 (m, 7H), 8.0 (d, 1H).

### EXAMPLE 158

### 2,3-Dimethylphenyl 4-[(1R,2S)-2-({[(1S)-1-(4-aminophenyl)ethyl]amino}-carbonyl)cyclohexyl]benzyl(cyclopropyl)carbamate

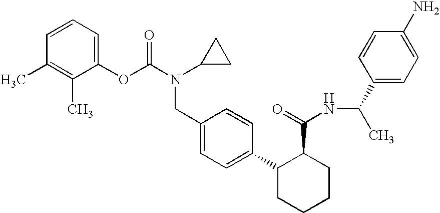

270 mg (0.47 mmol) of 2,3-dimethylphenyl cyclopropyl{4-[(1R,2S)-2-({[(1S)-1-(4-nitrophenyl)ethyl]amino}carbonyl)cyclohexyl]benzyl carbamates and Pd/C (about 20 mg) in 7 ml of ethyl acetate are stirred under hydrogen (1 atm) at room temperature for 14 h. After filtration and removal of the solvent, the crude product is purified by HPLC, yield: 170 mg (66%).

R_{f}=0.30 (cyclohexane/ethyl acetate 1:1); HPLC (Method 3): rt=4.59 min; ¹H NMR (200 MHz, DMSO): 0.85 (m, 4H), 1.15 (d, 3H), 1.2-1.6 (m, 4H), 1.7-1.9 (m, 4H), 2.0 (s, 3H), 2.3 (s, 3H), 2.6-2.8 (m, 2H), 4.5 (m, 2H), 4.65 (s, 2H), 6.3 (d, 2H), 6.4 (d, 2H); 6.9 (m, 1H); 7.0-7.2 (m, 6H), 7.65 (d, 2H).

### EXAMPLE 159

### 2,3-Dimethylphenyl 4-{(1R,2S)-2-[({(1S)-1-[4-(acetylamino)phenyl]ethyl}-amino)carbonyl]cyclohexyl}benzyl(cyclopropyl)carbamate

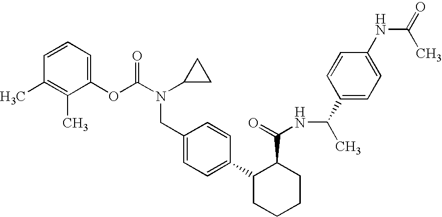

A solution of 50 mg (0.09 mmol) of 2,3-dimethylphenyl 4-[(1R,2S)-2-({[(1S)-1-(4-aminophenyl)ethyl]amino}carbonyl)cyclohexyl]benzyl(cyclopropyl)carbamate, 8.7 mg (0.11 mmol) of acetyl chloride and 14.4 mg (0.11 mmol) of diisopropyl-ethylamine in 2 ml of dichloromethane is stirred at room temperature for 14 h. The reaction mixture is concentrated and the residue is purified directly by HPLC, yield: 33 mg (61%).

HPLC (Method 3): rt=4.86 min; ¹H NMR (200 MHz, DMSO): 0.85 (m, 4H), 1.15 (d, 3H), 1.2-1.6 (m, 4H), 1.7-1.9 (m, 4H), 1.95 (s, 3H), 2.05 (s, 2H), 2.25 (s, 3H, 2.5-2.6 (m, 2H), 2.6-2.8 (m, 2H), 4.5-4.7 (m, 3H), 6.5 (d, 2H), 6.85 (bs, 1H), 7.0-7.1 (m, 2H), 7.1-7.3 (m, 6H), 7.8 (d, 1H).

The compounds listed in the following table are prepared in analogy to the method of Example 159.

| Example | | | HPLC method: |
|---|---|---|---|
| number | Structure | Mass | Retention time |
| 160 | 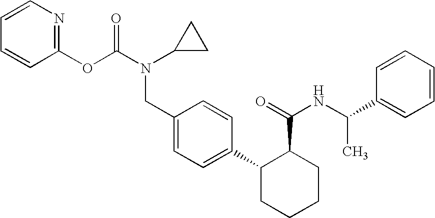 | MS (ESIpos): 498 (M + H)⁺ | Method 3: 4.63 min |
| 161 | 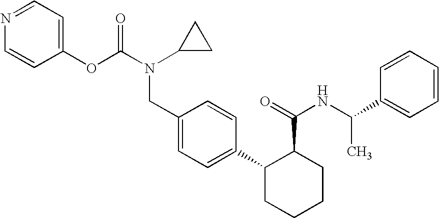 | MS (ESIpos): 498 (M + H)⁺ | Method 3: 4.29 min |
| 162 | 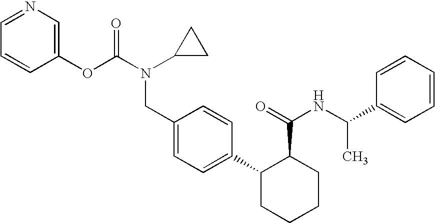 | MS (ESIpos): 498 (M + H)⁺ | Method 3: 4.31 min |
| 163 | 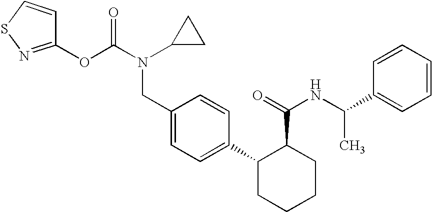 | MS (ESIpos): 526 (M + Na)⁺ | Method 3: 4.72 min |
| 164 | 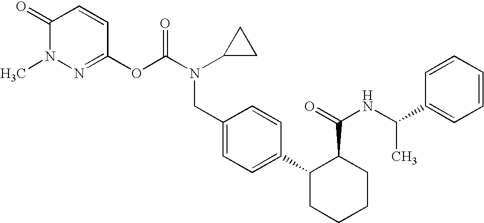 | MS (ESIpos): 529 (M + H)⁺ | Method 3: 4.41 min |
| 165 | 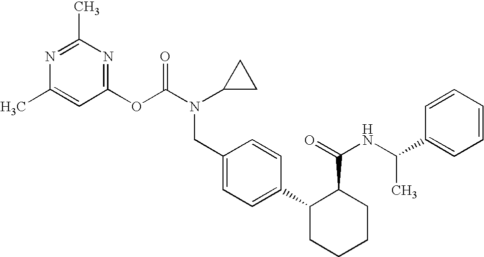 | MS (ESIpos): 527 (M + H)⁺ | Method 3: 4.35 min |
| 166 | 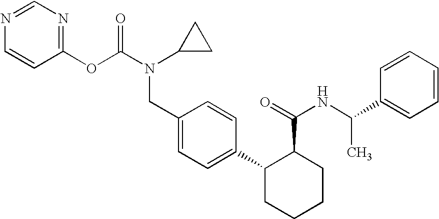 | MS (ESIpos): 499 (M + H)⁺ | Method 6: 4.13 min |

The compounds listed in the following table are prepared by general method [C.2] from the corresponding (1S,2R)-2-(4-{[cyclopropyl(aryloxycarbonyl)amino]-methyl}phenyl)cyclohexanecarboxylic acid:

| Example | | | HPLC method: |
|---|---|---|---|
| number | Structure | Mass | retention time |
| 167 | 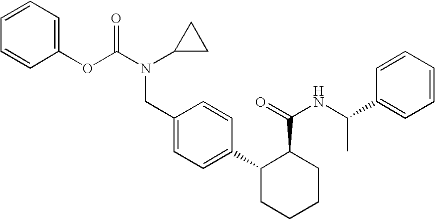 | MS (DCI, NH3): m/z = 514 [M + NH4]⁺ | Method 3: 5.24 min |
| 168 | 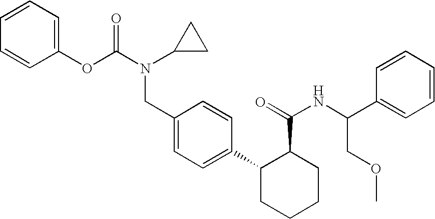 | MS (ESIpos): m/z =527 [M + H]⁺ | Method 3: 5.04 min |
| 169 | 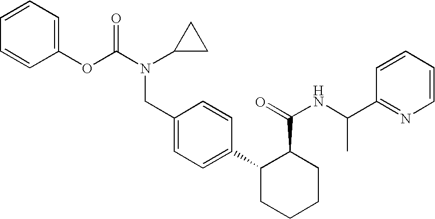 | MS (ESIpos): m/z =498 [M + H]⁺ | Method 3: 4.36 min |
| 170 | 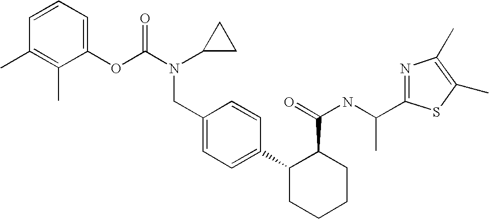 | MS (ESI): m/z =560 [M + H]⁺ | Method 10: 3.36 min |
| 171 | 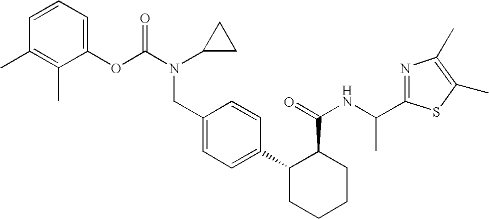 | MS (ESI): m/z =560 [M + H]⁺ | Method 10: 3.48 min |
| 172 | 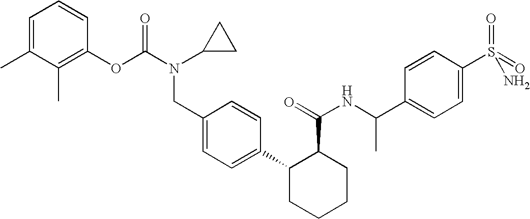 | MS (ESIpos): m/z =626 [M + Na]⁺ | Method 3: 4.96 min |
| 173 | 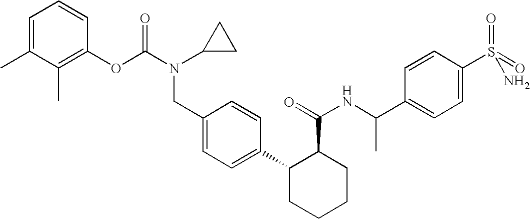 | MS (ESIpos): m/z =626 [M + Na]⁺ | Method 3: 4.99 min |
| 174 | 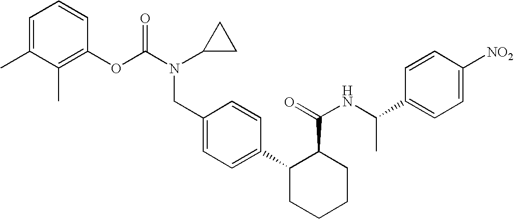 | MS (ESIpos): m/z =570 [M + H]⁺ | Method 3: 5.07 min |
| 175 | 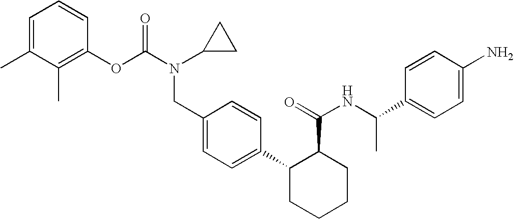 | MS (ESIpos): m/z =540 [M + H]⁺ | Method 3: 4.59 min |
| 176 | 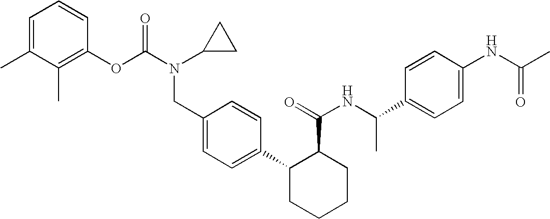 | MS (ESIpos): m/z =604 [M + Na]⁺ | Method 3: 4.86 min |

### EXAMPLE 177

### N-Methyl-N-(1-methyl-1-phenylethyl)-4-[(1R,2S)-2-({[(1S)-1-phenylethyl]-amino}carbonyl)cyclohexyl]benzamide

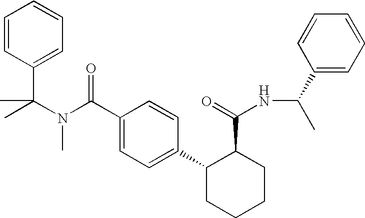

N,N-Diisopropylethylamine (42 μl, 0.24 mmol) and N-methyl-N-(1-methyl-1-phenylethyl)amine [Synthesis 1995, 1534-1538] (44.6 mg, 0.3 mmol) are added at room temperature to a solution of 4-[(1R,2S)-2-({[(1S)-1-phenylethyl]amino}-carbonyl)cyclohexyl]benzoic acid (70.0 mg, 0.2 mmol), PyBOP (124.4 mg, 0.24 mmol) and 4-dimethylaminopyridine (2.4 mg, 0.02 mmol) in dimethylformamide (3 ml). The reaction mixture is stirred at room temperature for 16 h. The desired product is purified by HPLC chromatography (acetonitrile/water mixtures).

MS (DCI, NH₃): m/z=484 [M+H]⁺, 500 [M+NH₄]⁺; HPLC (Method 3): rt=4.26 min; ¹H NMR (200 MHz, DMSO): 7.99 (d, 1H), 7.49-7.10 (m, 9H), 7.02-6.88 (m, 3H), 6.59 (dd, 2H), 4.76-4.58 (m, 1H), 2.95 (s, 3H), 2.82-2.68 (m, 1H), 2.64-2.52 (m, 1H), 1.90-1.28 (m, 14H), 1.20 (d, 3H).

The following compounds are prepared in analogy to general method [F]:

| Example | | | HPLC method: |
|---|---|---|---|
| number | Structure | Mass | retention time |
| 178 | 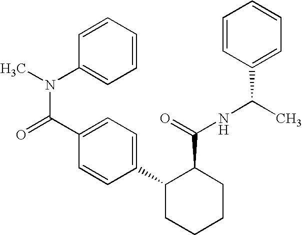 | MS (ESI): m/z =441 [M + H]⁺ | Method 3: 4.20 min |
| 179 | 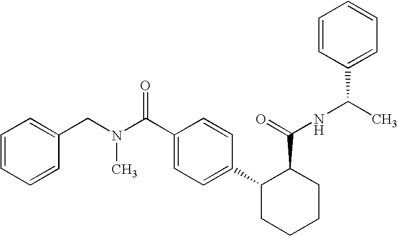 | MS (DCI, NH₃): m/z = 455 [M + H]⁺, 472 [M + NH₄]⁺ | Method 3: 4.75 min |
| 180 | 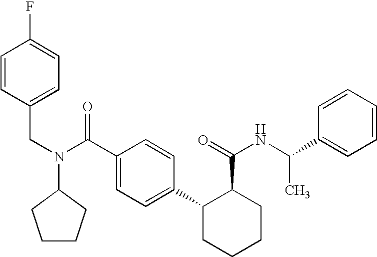 | MS (ESI): m/z =527 [M + H]⁺ | Method 3: 5.26 min |
| 181 | 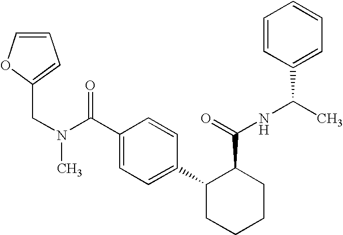 | MS (DCI, NH₃): m/z = 446 [M + H]⁺, 462 [M + NH₄]⁺ | Method 3: 4.56 min |
| 182 | 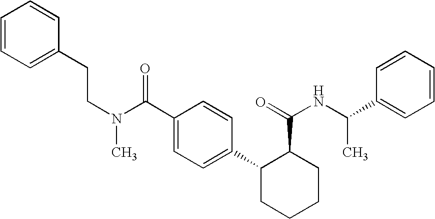 | MS (ESI): m/z =469 [M + H]⁺ | Method 3: 4.78 min |
| 183 | 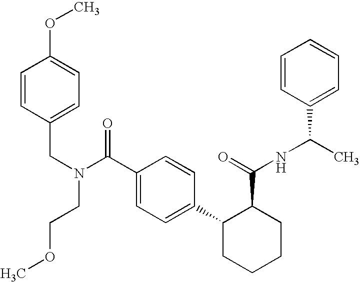 | MS (ESI): m/z =529 [M + H]⁺ | Method 3: 4.73 min |
| 184 | 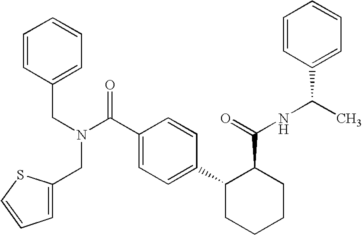 | MS (DCI, NH₃): m/z = 538 [M + H]⁺, 554 [M + NH₄]⁺ | Method 3: 5.17 min |
| 185 | 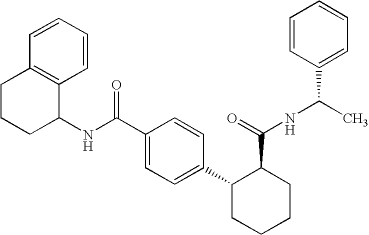 | MS (DCI, NH₃): m/z = 481 [M + H]⁺, 498 [M + NH₄]⁺ | Method 3: 4.79 min |
| 186 | 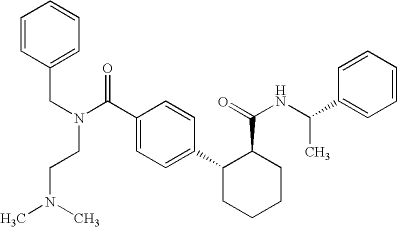 | MS (DCI, NH₃): m/z = 512 [M + H]⁺ | Method 3: 4.24 min |
| 187 | 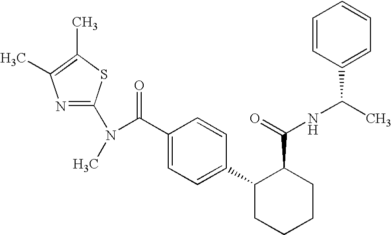 | MS (ESI): m/z (%) =476 [M + H]⁺ | Method 3: 4.66 min |
| 188 | 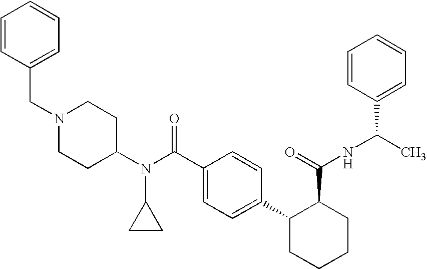 | MS (ESI): m/z =564 [M + H]⁺ | Method 3: 4.38 min |
| 189 | 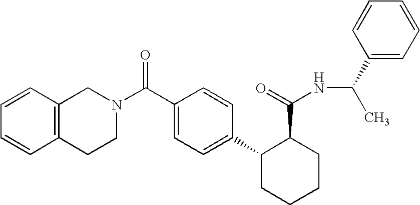 | MS (DCI, NH₃): m/z = 467 [M + H]⁺, 484 [M + NH₄]⁺ | Method 3: 4.70 min |
| 190 | 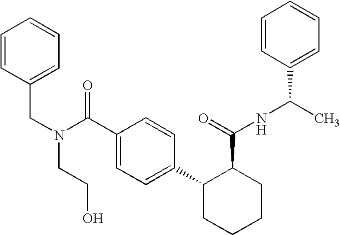 | MS (ESI): m/z =485 [M + H]⁺ | Method 3: 4.36 min |
| 191 | 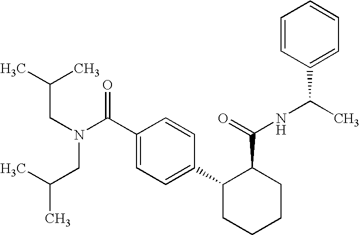 | MS (DCI, NH₃): m/z = 463 [M + H]⁺, 480 [M + NH₄]⁺ | Method 3: 4.89 min |
| 192 | 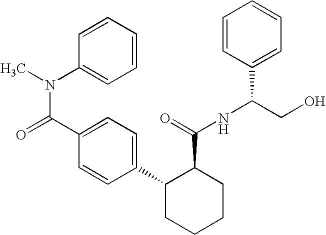 | MS (ESI): m/z =457 [M + H]⁺ | Method 3: 4.75 min |
| 193 | 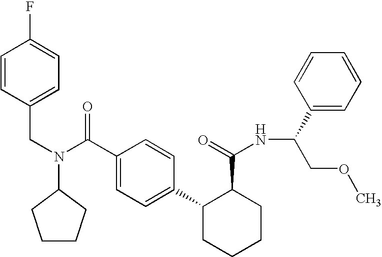 | MS (ESI): m/z =557 [M + H]⁺ | Method 3: 5.25 min |
| 194 | 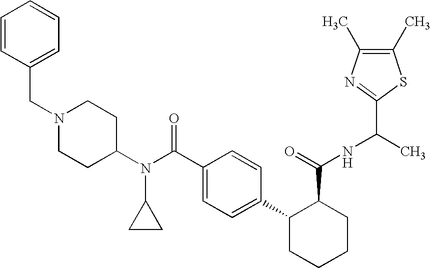 | MS (ESI): m/z =599 [M + H]⁺ | Method 10: 1.87 min, 1.97 min |
| 195 | 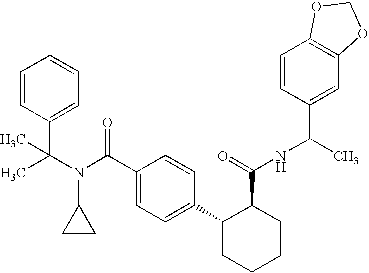 | MS (ESI): m/z =527 [M + H]⁺ | Method 10: 3.20 min |

### EXAMPLE 196

### (1S*,2R*)-2-(3-{[Methyl(1-methyl-1-phenylethyl)amino]methyl}phenyl)-N-[(1S)-1-phenylethyl]cyclohexanecarboxamide

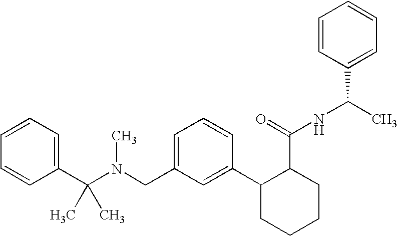

a) 78 mg (0.52 mmol) of N-methyl-2-phenyl-2-propanamine and 200 mg (0.64 mmol) of methyl (1S*,2R*)-2-[3-(bromomethyl)phenyl]cyclohexane-carboxylate (Example L) are reacted by general method [D] to give the corresponding tertiary amine, yield 142 mg (72%).

b) 130 mg (0.34 mmol) of methyl (1S*,2R*)-2-(3-{[methyl(1-methyl-1-phenylethyl)amino]methyl}phenyl)cyclohexanecarboxylate and 27.4 mg (0.69 mmol) of sodium hydroxide in 2.5 ml of ethanol and 0.25 ml of water are heated under reflux for 24 h. For workup, the mixture is adjusted to pH 1 with aqueous hydrochloric acid, the solvents are stripped off, and the residue is purified by HPLC; yield:. 90 mg (72%).

c) 73 mg (0.20 mmol) of (1S*,2R*)-2-(3-{[methyl(1-methyl-1-phenylethyl)-amino]methyl}phenyl)cyclohexanecarboxylic acid and 36 mg (0.30 mmol) of (S)-phenylethylamine are reacted by general method [C] to give the title compound, yield: 54 mg (58%) as mixture of diastereomers.

R_{f}=0.13 (dichloromethane/methanol 50:1); MS (DCI): m/z=469 (M+H)⁺; ¹H NMR (400 MHz, DMSO): 0.8 (d, 3H), 1.2 (d, 3H), 1.3-1.6 (m, 14H), 1.6-1.9 (m, 8H), 1.95 (s, 3H), 2.1 (s, 3H), 2.5 (m, 1H), 2.7 (m, 1H), 4.5-4.7 (m, 2H), 6.5 (d, 2H), 6.9-7.4 (m, 18H), 7.6 (d, 4H), 7.8 (d, 1H), 7.9 (d, 1H).

The diastereomers are isolated by preparative HPLC [column: Stability C30; 5 μm; 250* 20 mm; temperature: 40° C.; flow rate=25 ml min⁻¹; eluent composition: A=0.2% strength trifluoroacetic acid, B=acetonitrile; isocratic 55% A and 45% B (v/v)]:

diastereomer A, yield 18 mg (43%); HPLC retention time (Method 12): 11.83 min.

diastereomer B, yield 20 mg (47%); HPLC retention time (Method 12); 14.61 min.

The compounds listed in the following table are prepared in analogy to the method of Example 196.

| Example | | | HPLC method: | |
|---|---|---|---|---|
| number | Structure | Mass | retention time | Diastereomer |
| 197 | 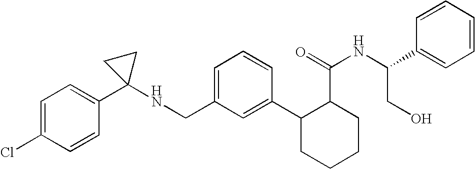 | MS (ESIpos): 503 (M + H)⁺ | Method 3: 4.52 min | B |
| 198 | 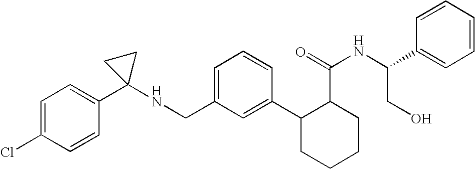 | MS (ESIpos): 503 (M + H)⁺ | Method 3: 4.29 min | A |
| 199 | 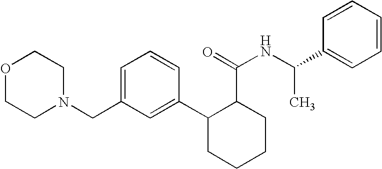 | MS (ESIpos): 407 (M + H)⁺ | Method 3: 4.14 min | B |
| 200 | 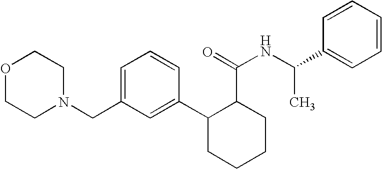 | MS (ESIpos): 407 (M + H)⁺ | Method 3: 4.09 min | A |
| 201 | 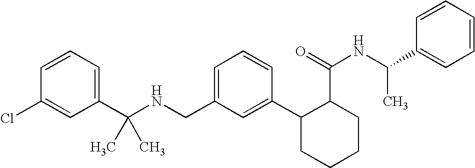 | MS (ESIpos): 489 (M + H)⁺ | Method 3: 4.56 min, 4.63 min | A + B |
| 202 | 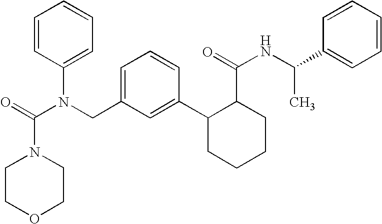 | MS (ESIpos): 526 (M + H)⁺ | Method 3: 4.74 min, 4.81 min | A + B |
| 203 | 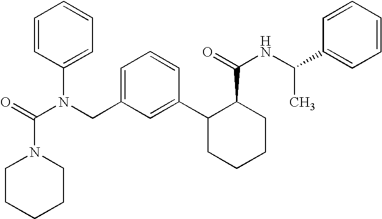 | MS (ESIpos): 524 (M + H)⁺ | Method 3: 5.22 min, 5.28 min | A + B |

### EXAMPLE 204

### Phenyl cyclopropyl(4-{(1S,2S)-2-[({[(1S)-1-phenylethyl]amino}carbonyl)-amino]cyclohexyl}benzyl)carbamate

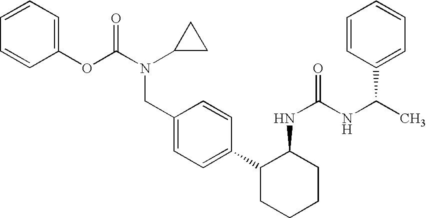

a) A solution of 90 mg (0.66 mmol) of isobutyl chloroformate in 1 ml of acetone is added dropwise to a solution of 200 mg (0.51 mmol) of (1S,2R)-2-(4-{[cyclopropyl(phenoxycarbonyl)amino]methyl}phenyl)cyclohexanecarboxylic acid, 61.7 mg (0.61 mmol) of triethylamine in 3 ml of acetone at −10° C. The reaction mixture is stirred at −10° C. for a further 1 h and, after addition of a solution of 49.6 mg (0.76 mmol) of sodium azide in 0.5 ml of water, stirred at −10° C. for a further 2 h. The reaction mixture is added to ice-water, and the aqueous phase is extracted with diethyl ether. The ether extracts are dried over sodium sulfate and concentrated at low temperature (<30° C.). The residue is dropped into 2 ml of toluene at 110° C., and the reaction mixture is heated under reflux for a further 2.5 h.

b) 1 ml of the toluene solution is mixed with 31 mg (0.26) of (S)-phenylethylamine and stirred at room temperature for 14 h. The reaction mixture is concentrated and purified by HPLC, yield 36 mg (28%).

R_{f}=0.13 (57 (cyclohexane/ethyl acetate 7:3); MS (ESIpos)=512 (M+H)⁺; ¹H NMR (200 MHz, DMSO): 0.7 (m, 4H), 1.1 (d, 3H), 1.1-1.6 (m, 4H), 1.6-1.95 (m, 4H), 2.4-2.5 (m, 1H), 2.7 (m, 1H), 3.6 (m, 1H), 4.4-4.6 (m, 3H), 5.5 (d, 1H), 5.95 (d, 1H), 7.0-7.3 (m, 12H), 7.4-7.5 (m, 2H).

The following compound is prepared in analogy to the method of Example 204:

| Example | | | HPLC method: |
|---|---|---|---|
| number | Structure | Mass | retention time |
| 205 | 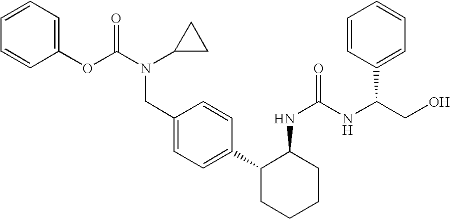 | MS (ESIpos): 528 (M + H)⁺ | Method 3: 4.84 min |

### Reaction of (1S,2R)-2-{4-[(benzyloxy)carbonyl]phenyl}cyclohexanecarboxylic acids to give amides

The compounds listed in the following table are prepared by general method [C-2] from the corresponding (1S,2R)-2-{4-[(benzyloxy)carbonyl]phenyl}cyclo-hexanecarboxylic acids.

| Example | | | HPLC method: |
|---|---|---|---|
| number | Structure | Mass | retention time |
| 206 | 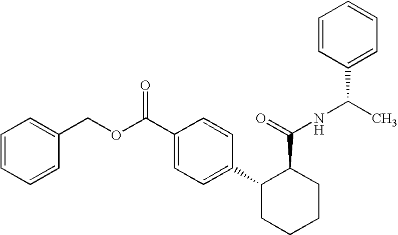 | MS (ESI): m/z = 442 [M + H]⁺ | Method 3: 5.08 min |
| 207 | 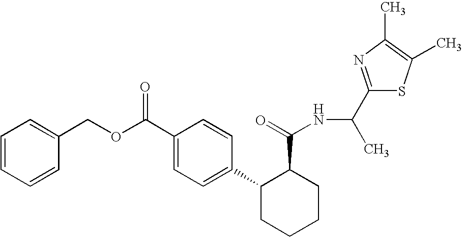 | MS (ESI): m/z = 477 [M + H]⁺ | Method 3: 4.66 min |
| 208 | 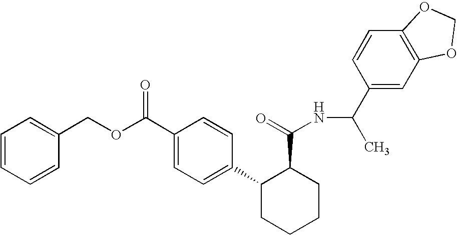 | MS (DCI, NH3): m/z = 486 [M + H]⁺, 503 [M + NH₄]⁺ | Method 3: 4.93 min, 4.99 min |
| 209 | 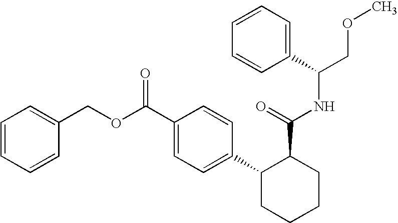 | ES (ESI): m/z = 472 [M + H]⁺ | Method 3: 4.90 min |

### EXAMPLE 210

### (1S,2S)-2-(4-[Phenoxymethyl]phenyl)-N-[(1S)-1-phenylethyl]cyclohexane-carboxamide

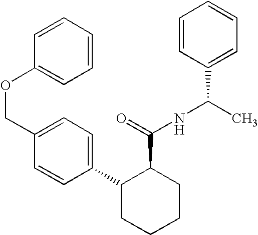

### a) tert-Butyl (1S,2S)-2-(4-[phenoxymethyl]phenyl)cyclohexanecarboxylate

The compound is synthesized by general method [H] starting from phenol. The residue is purified by flash chromatography on silica gel (cyclohexane/EtOAc 7:1). 1.1 g (88% yield) of the desired compound are obtained.

R_{f}=0.20 (petroleum ether/ethyl acetate 7:1); ¹H NMR (300 MHz, DMSO): 1.08 (s, 9H), 1.42 (m, 4H), 1.78 (m, 4H), 2.62 (m, 2H), 5.08 (s, 2H), 7.15 (m, 14H).

### b) (1S,2S)-2-(4-[Phenoxymethyl]phenyl)cyclohexanecarboxylic acid

The compound is synthesized starting from the corresponding tert-butyl ester derivative by general hydrolysis method [B]. 0.8 g (HPLC purity: 97% yield) of the desired compound is obtained and is reacted without further purification.

R_{f}=0.23 (petroleum ether/ethyl acetate 1:1); ¹H NMR (300 MHz, DMSO): 1.43 (m, 4H), 1.76 (m, 4H), 2.64 (m, 2H), 5.02 (s, 2H), 7.15 (m, 14H), 8.80 (s, 1H).

### c) (1S,2S)-2-(4-[Phenoxymethyl]phenyl)-N-[(1S)-1-phenylethyl]cyclo-hexanecarboxamide

The compound is synthesized starting from (1S,2S)-2-(4-[phenoxymethyl]phenyl)-cyclohexanecarboxylic acid and (S)-phenylethylamine by general synthesis method [C] for amide coupling. 130 mg (yield 89%) of the desired compound are obtained.

R_{f}=0.72 (dichloromethane/methanol 10:1); ¹H NMR (300 MHz, DMSO): 1.20 (d, 3H), 1.37 (m, 4H), 1.76 (m, 4H), 2.65 (m, 2H), 4.68 (m, 1H), 5.08 (s, 2H), 7.15 (m, 14H), 8.00 (d, 1H).

The compounds listed in the following table are prepared in analogy to the method of Example 210.

| Example | | | HPLC method: |
|---|---|---|---|
| number | Structure | Mass | retention time |
| 211 | 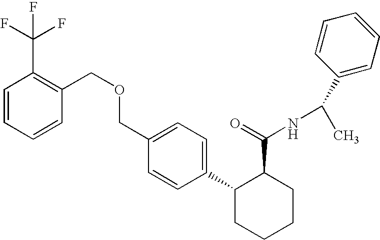 | MS (ESI): m/z = 496 [M + H]⁺ | Method 3: 4.85 min |
| 212 | 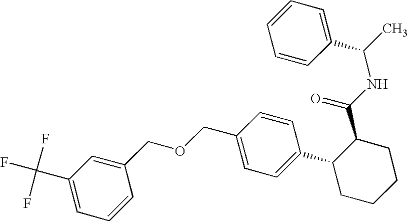 | MS (ESI): m/z = 496 [M + H]⁺ | Method 3: 4.88 min |
| 213 | 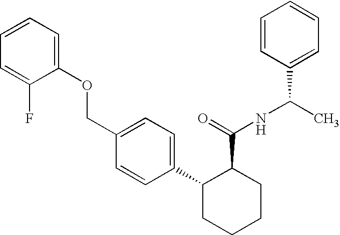 | MS (ESI): m/z = 432 [M + H]⁺ | Method 3: 4.82 min |
| 214 | 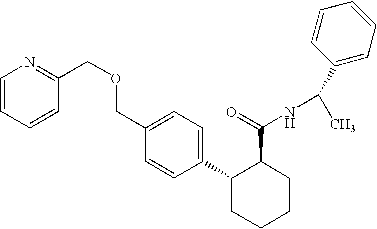 | MS (ESI): m/z = 429 [M + H]⁺ | Method 3: 4.13 min |
| 215 | 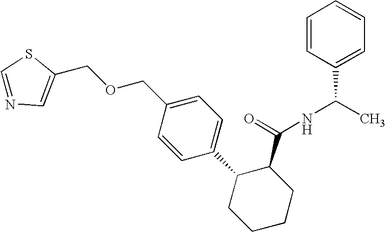 | MS (ESI): m/z = 436 [M + H]⁺ | Method 3: 4.20 min |
| 216 | 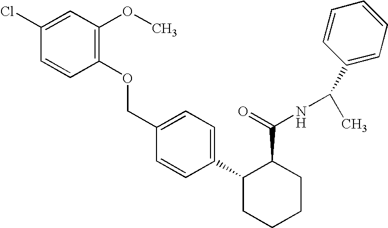 | MS (ESI): m/z = 479 [M + H]⁺ | Method 3: 4.62 min |
| 217 | 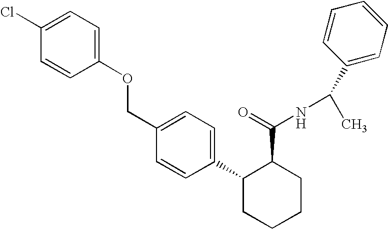 | MS (ESI): m/z = 449 [M + H]⁺ | Method 3: 4.55 min |
| 218 | 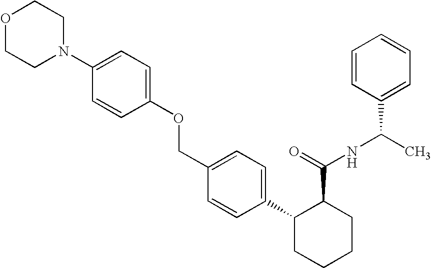 | MS (ESI): m/z = 500 [M + H]⁺ | Method 3: 4.05 min |

### EXAMPLE 219

### (1S*,2R*)-2-[3-(Phenoxymethyl)phenyl]-N-[(1S)-1-phenylethyl]cyclohexane-carboxamide

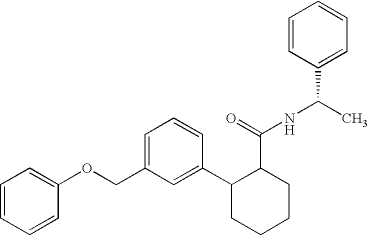

a) 66.5 mg (0.71 mmol) of phenol and 200 mg (0.64 mmol) (1S*,2R*)-2-[3-(bromomethyl)phenyl]cyclohexanecarboxylate are reacted by general method [D] to give the corresponding ether, yield 104 mg (78%).

b) 135 mg (0.42 mmol) of methyl (1S*,2R*)-2-[3-(phenoxymethyl)phenyl]-cyclohexanecarboxylate and 33 mg (0.83 mmol) of sodium hydroxide in 2.5 ml of ethanol and 0.25 ml of water are heated under reflux for 24 h. For workup, the mixture is adjusted to pH 1 with aqueous hydrochloric acid, the solvents are stripped off, and the residue is purified by HPLC, yield; 125 mg (97%).

c) 100 mg (0.32 mmol) of (1S*,2R*)-2-[3-(phenoxymethyl)phenyl]cyclo-hexanecarboxylic acid and 58 mg (0.48 mmol) of (S)-phenylethylamine are reacted by general method [C] to give the title compound, yield: 104 mg (79%) as mixture of diastereomers.

R_{f}=0.57 (cyclohexane/ethyl acetate 1:1). MS (DCI): m/z=431 [M+NH₄]⁺

¹H NMR(400 MHz, DMSO): 0.9 (d, 3H), 1.2 (d, 3H), 1.3-1.55 (m, 8H), 1.7-1.9 (m, 8H), 2.5-2.65 (m, 2H), 2.75 (t, 2H), 4.6 (g, 1H), 4.65 (g, 1H), 4.92 (d, 1H), 4.95 (d, 1H), 5.0 (d, 1H), 6.6 (m, 2H), 6.9-7.1 (m, 7H), 7.1-7.2 (m, 5H), 7.2-7.3 (m, 12H), 7.85 (d, 1H, 7.95 (d, 1H).

The diastereomers are isolated by preparative HPLC [column: Stability C30; 5 μm; 250* 20 mm; temperature: 40° C.; flow rate=25 ml min⁻¹; eluent composition: A=0.2% strength trifluoroacetic acid, B=acetonitrile; isocratic 55% A and 45% B (v/v)]:

diastereomer A, yield 18 mg (43%); HPLC retention time (Method 12): 6.67 min.

diastereomer B, yield 20 mg (47%); HPLC retention time (Method 12); 7.53 min.

The compounds listed in the following table are prepared in analogy to the method of Example 219.

| | | | | |
|---|---|---|---|---|
| Example | | | HPLC method: | |
| number | Structure | Mass | retention time | Diastereomer |

| | | | | |
|---|---|---|---|---|
| 220 | 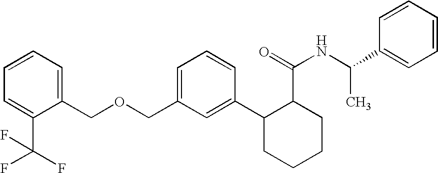 | MS (DCI): 513 [M + NH₄]⁺ | Method 3: 5.35 min, 5.39 min | A + B |
| 221 | 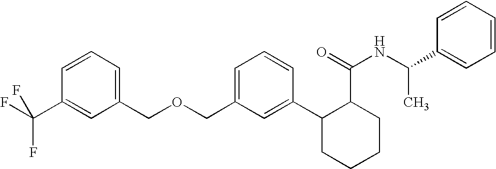 | MS (DCI): 513 [M + NH₄]⁺ | Method 3: 5.35 min | A + B |
| 222 | 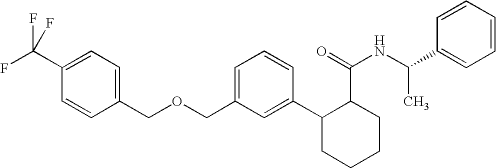 | MS (DCI): 513 [M + NH₄]⁺ | Method 3: 5.35 min | A + B |
| 223 | 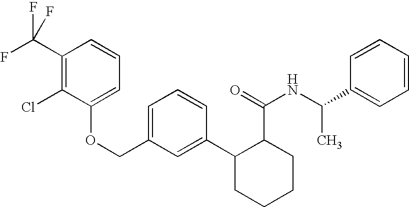 | MS (ESIpos): 516 [M + H]⁺ | Method 3: 5.53 min | A + B |

| Example | | | HPLC method: | | BAY |
|---|---|---|---|---|---|
| number | Structure | Mass | retention time | | No. |
| 224 | 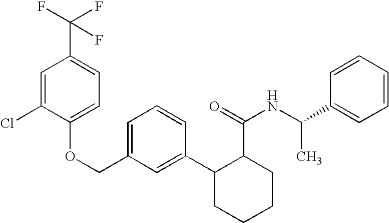 | MS (DCI): 534 [M + NH₄]⁺ | Method 3: 5.61 min | A + B | BAY 659521 |
| 225 | 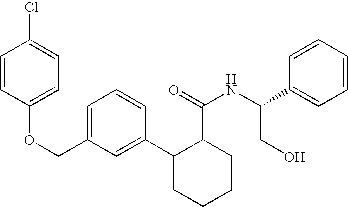 | MS (DCI): 465 [M + NH₄]⁺ | Method 3: 5.43 min | A + B | BAY 659527 |
| 226 | 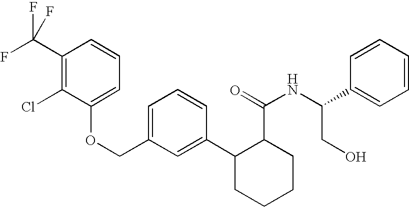 | MS (ESIpos): 532 [M + H]⁺ | Method 3: 5.12 min | A | BAY 659534 |
| 227 | 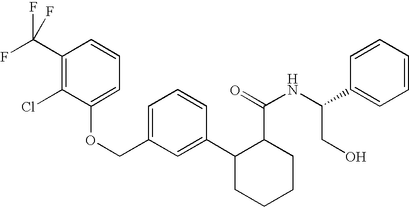 | MS (ESIpos): 532 [M + H]⁺ | Method 3: 5.19 min | B | BAY 648985 |

### EXAMPLE 228

### (1S*,2R*)-2-[4-(Benzyloxy)phenyl]-N-[(1S)-1-phenylethyl]cyclohexanecarbox-amide

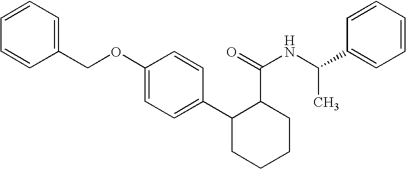

Benzyl bromide (25.7 μl, 0.22 mmol) is added to a suspension of sodium hydride (60% in mineral oil, 8.4 mg, 0.22 mmol) and (1S,2R)-2-(4-hydroxyphenyl)-N-[(1S)-1-phenylethyl]cyclohexanecarboxamide (65.4 mg, 0.18 mmol) in tetrahydro-furan (2 ml) at room temperature. The reaction mixture is stirred for 20 h and water and dichioromethane are added. After phase separation, the aqueous phase is extracted with dichioromethane, and the combined organic phases are dried (sodium sulfate), filtered and concentrated in vacuo.

The desired product is purified by HPLC chromatography (acetonitrile/water mixtures). Yield: 51% as mixture of diastereomers.

MS (ESI): m/z=414 [M+H]⁺; HPLC (Method 3): rt=5.04 min; ¹H NMR (200 MHz, DMSO): 7.87 (d, 1H), 7.78 (d, 1H), 7.50-6.98 (m, 22H), 6.91-6.84 (m, 4H), 6.65-6.60 (m, 2H), 5.07 (s, 2H), 5.06 (s, 2H), 4.75-4.56 (m, 2H), 2.70-2.59 (m, 2H), 2.47-2.39 (m, 2H), 1.86-1.67 (m, 8H), 1.59-1.28 (m, 8H), 1.22 (d, 3H), 0.90 (d, 3H).

The compounds listed in the following table are prepared in analogy to the method of Example 228.

| Example | | | HPLC method: |
|---|---|---|---|
| number | Structure | Mass | retention time |
| 229 | 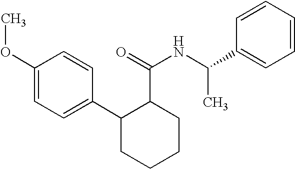 | MS (DCI, NH3): m/z = 338 [M + H]⁺, 355 [M + NH₄]⁺ | Method 3: 4.69 min, 4.75 min |
| 230 | 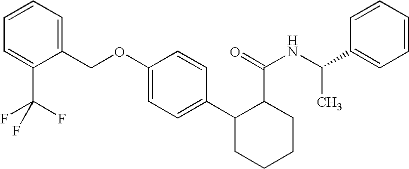 | MS (ESI): m/z = 482 [M + H]⁺ | Method 3: 5.18 min, 5.22 min |
| 231 | 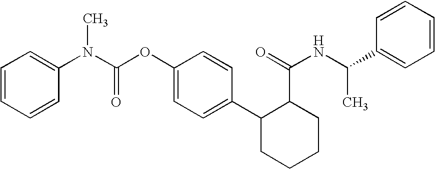 | MS (DCI, NH3): m/z = 458 [M + H]⁺, 474 [M + NH₄]⁺ | Method 3: 5.28 min, 5.32 min |
| 232 | 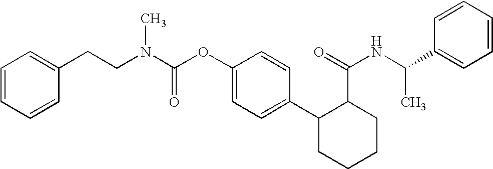 | MS (ESI): m/z = 485 [M + H]⁺ | Method 6: 4.76 min |
| 233 | 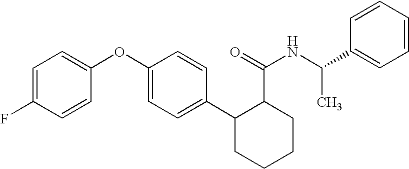 | MS (DCI, NH3): m/z = 418 [M + H]⁺, 435 [M + NH₄]⁺ | Method 3: 5.15 min, 5.20 min |
| 234 | 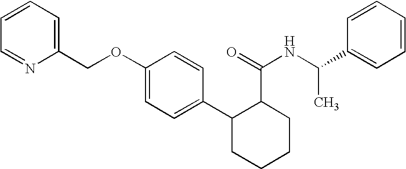 | MS (ESI): m/z = 415 [M + H]⁺ | Method 10: 2.33 min |
| 235 | 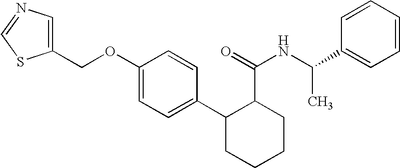 | MS (ESI): m/z = 421 [M + H]⁺ | Method 6: 4.21 min |
| 236 | 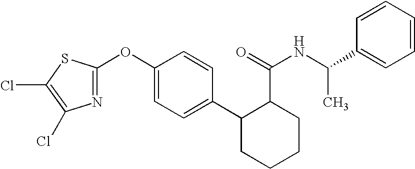 | MS (DCI, NH3): m/z = 458 [M + H]⁺, 492 [M + NH₄]⁺ | Method 3: 5.29 min |

General Method for Preparing Substituted Cyclohexane Derivatives of the Formula A by Solid Phase-Assisted Synthesis:

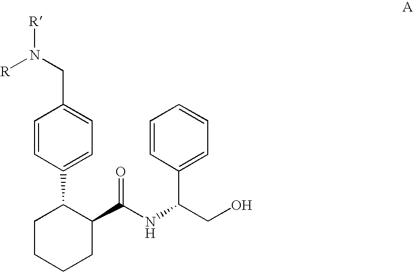

Attachment of (R)-Fmoc-Phenylglycinol to Chlorotrityl Polystyrene Resin

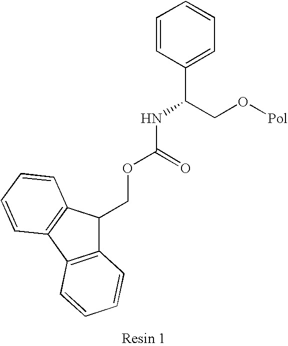

Chlorotrityl polystyrene (5.00 g, 4.90 mmol, Rapp Polymere) and (R)-Fmoc-phenylglycinol (2.6 g, 7.3 mmol) are suspended in toluene/pyridine (4:1) and stirred at 50° C. for three hours. Methanol (5 ml) is added, and the mixture is stirred at 50° C. for a further three hours. The reaction mixture is filtered and the resulting Resin 1 is repeatedly washed with methanol, dichloromethane and diethyl ether and dried. To determine the loading, trifluoroacetic acid/dichloromethane is used for elimination from a resin sample. A loading of 0.98 mm/g (R)-Fmoc-phenylglycinol is determined by quantitative HPLC.

Cleavage of the Fmoc Protective Group

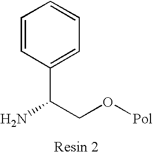

Resin 1 (100 mg) is shaken in piperidine/dimethylformamide (1:4, 1 ml) at room temperature for 20 minutes. The resulting Resin 2 is filtered and repeatedly washed with methanol, dichloromethane and diethyl ether and dried.

Amide Formation

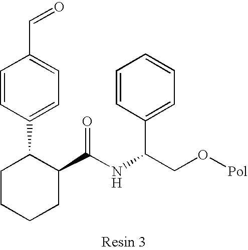

[1S,2S]-2-(4-Formylphenyl)cyclohexanecarboxylic acid (8.88 g, 38.2 mmol), ethyldiisopropylamine (19.8 g, 153 mmol) and TBTU (24.5 g, 76.4 mmol) are stirred in 250 ml of dichloromethane for 10 minutes. This solution is cooled in an ice bath, and Resin 2 (26.0 g) is added. The reaction mixture is mixed with dimethylacetamide (100 ml), warmed to room temperature and shaken for 3 hours. The reaction mixture is filtered and the resulting Resin 3 is repeatedly washed with dimethylformamide, methanol, dichloromethane and diethyl ether and dried.

Preparation of a Library of Substituted Cyclohexane Derivatives of the Formula A

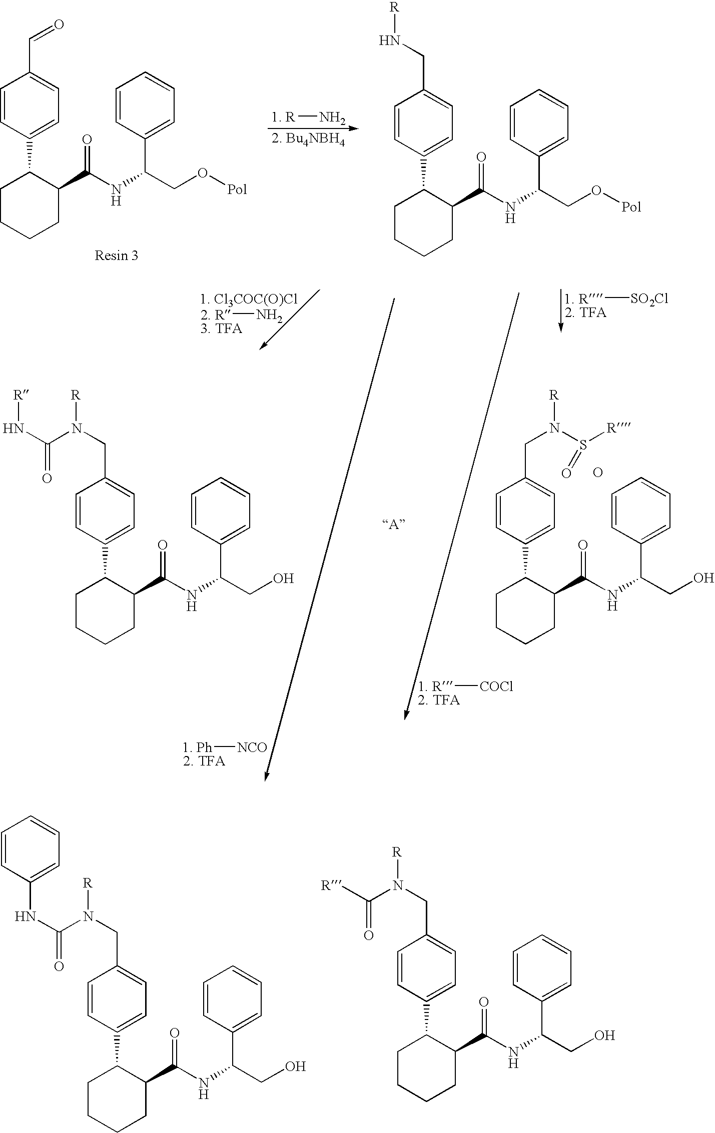

The library was prepared in MiniKans (IRORI) by the mix and split method [K. C. Nicolaou, X.-Y. xiao, Z. Parandoosh, A. Senyei, M. P. Nova, Angew. Chem. Int. Ed. Engl. (1995), 35, 2289-2290].

Resin 3 is slurried as a suspension in dichloromethane/dimethylformamide (2:1) in IRORI MiniKans (about 120 mg/Kan in each case) and repeatedly washed with dichloromethane and diethyl ether and dried.

Reductive Amination

The resin compartmentalized in this way is suspended in dichloro-methane/trimethyl orthoformate (1:1) in separate reaction vessels and, after addition of an amine (5 eq., “R—NH₂”) at room temperature, shaken for 18 hours. The resin is washed in the separate reaction vessels twice with dimethylformamide and suspended in dimethylformamide, and tetrabutylammonium borohydride (2 eq) is added at room temperature. After shaking at room temperature for 10 minutes, the reaction mixture is cooled to −40° C. and, after addition of glacial acetic acid (100 eq), warmed to room temperature again. The resin is repeatedly washed with water, methanol, dichloromethane/10% diisopropylethylamine, methanol, dichloromethane and diethyl ether and dried.

Synthesis of Ureas

Method 1:

The resin which has again been compartmentalized is suspended in dichloromethane in separate reaction vessels and, after addition of diisopropylethylamine (15 eq), cooled in an ice bath. Trichloromethyl chloroformate (5 eq) is added, and the mixture is stirred for 30 minutes. The reaction solution is decantered, the resin is washed once with dichloromethane, then a solution of primary or secondary amines (10 eq of each, “R″—NH₂”) and ethyldiisopropylamine (10 eq) in dimethylformamide is added, and the mixture is shaken at room temperature overnight.

Method 2:

The resin which has again been compartmentalized is suspended in dioxane and, after addition of phenyl isocyanate (10 eq) and dimethylaminopyridine (0.5 eq), shaken at 50° C. overnight.

Synthesis of Sulfonamides, Carbamates and Amides

The resin which has again been compartmentalized is suspended in dichloromethane in separate reaction vessels and, after addition of ethyldiisoprQpylamine (15 eq), at between 0° C. and room temperature acid chlorides, chlorofounic esters or sulfonyl chlorides (5 eq of each) are added and the mixture is shaken at 50° C. overnight.

The resin intermediates obtained in this way are finally washed repeatedly with methanol, dimethylformamide, water, dimethylformamide, methanol, dichloromethane and diethyl ether and dried. The products are then cleaved off the solid phase with trifluoroacetic acid/dichloromethane (1:1), the resin is filtered off, and the reaction solutions are evaporated.

The compounds obtained in this way are listed in the following table:

| | | | Retention |
|---|---|---|---|
| Example | Structure | Mass | time |
| 29 | 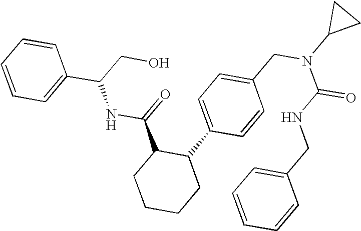 | 525.3 | 3.98 |
| 30 | 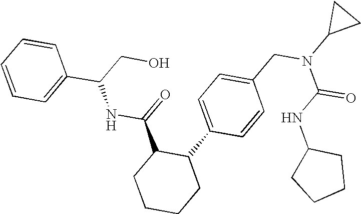 | 503.31 | 4.01 |
| 31 | 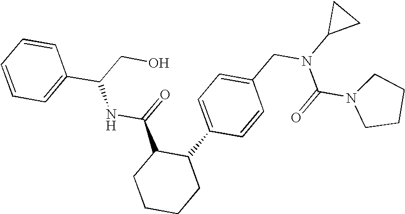 | 489.3 | 3.72 |
| 32 | 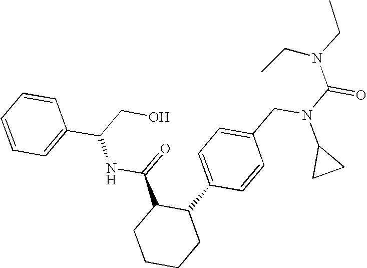 | 491.31 | 3.92 |
| 33 | 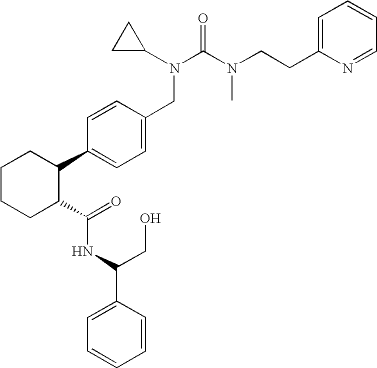 | 554.33 | 2.79 |
| 34 | 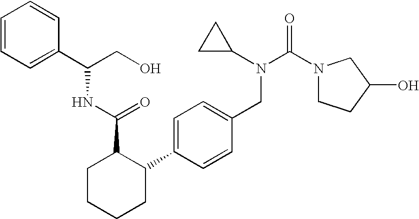 | 505.29 | 3.21 |
| 35 | 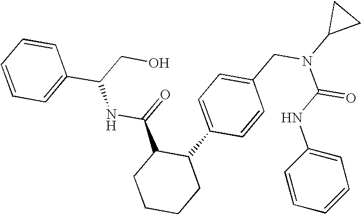 | 511.28 | 4.07 |
| 36 | 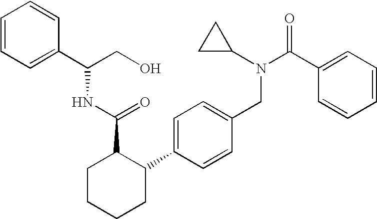 | 496.27 | 3.97 |
| 37 | 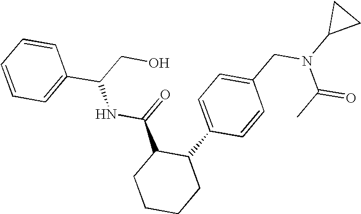 | 434.26 | 3.48 |
| 38 | 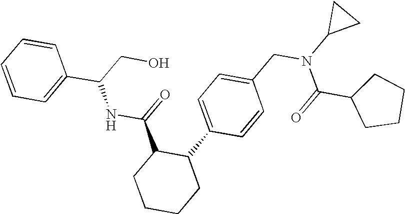 | 488.3 | 4.18 |
| 39 | 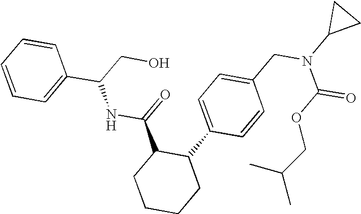 | 492.3 | 4.43 |
| 40 | 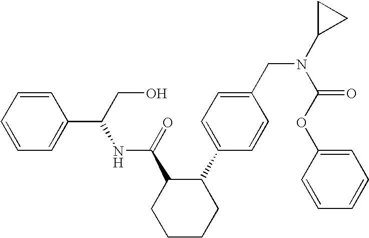 | 512.27 | 4.3 |
| 41 | 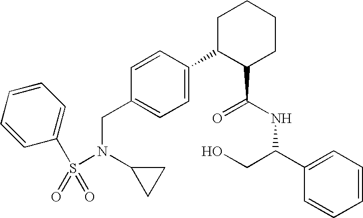 | 532.24 | 4.23 |
| 42 | 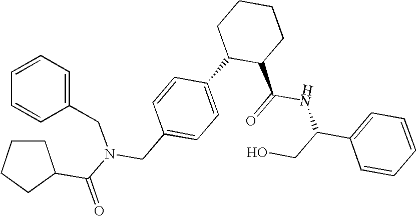 | 539.31 | 4.15 |
| 43 | 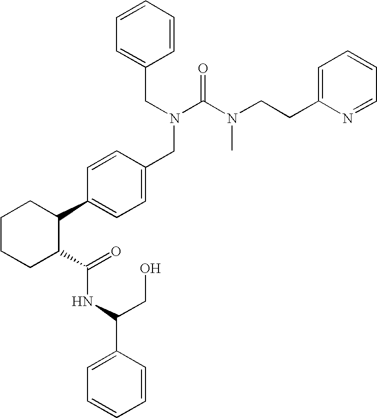 | 604.34 | 3.11 |
| 44 | 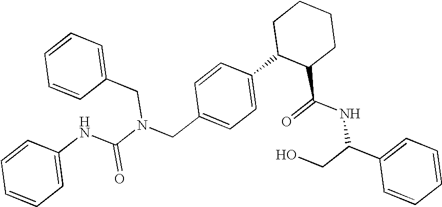 | 561.3 | 4.31 |
| 45 | 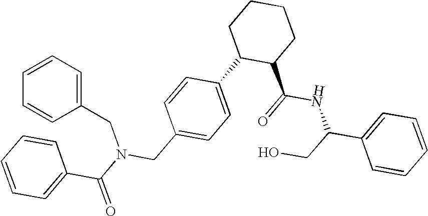 | 546.29 | 4.28 |
| 46 | 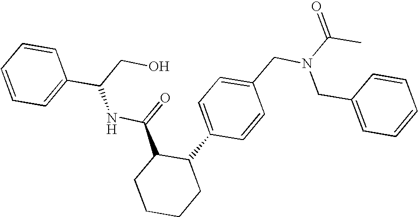 | 484.27 | 3.83 |
| 47 | 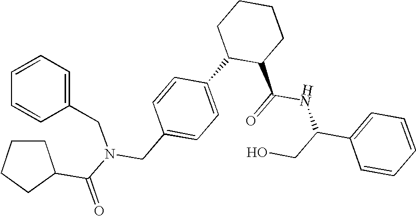 | 538.32 | 4.46 |
| 48 | 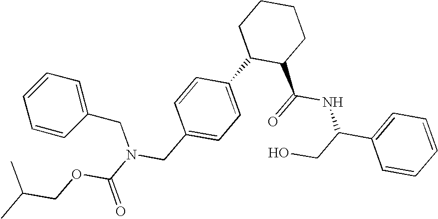 | 542.31 | 4.75 |
| 49 | 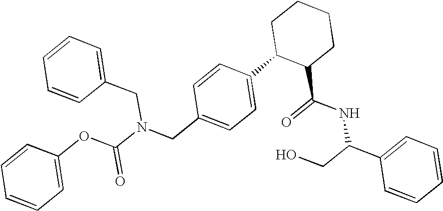 | 562.28 | 4.64 |
| 50 | 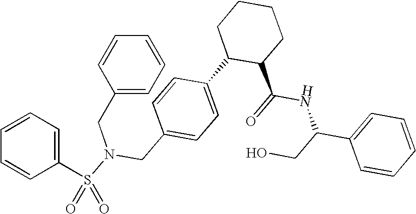 | 582.26 | 4.51 |
| 51 | 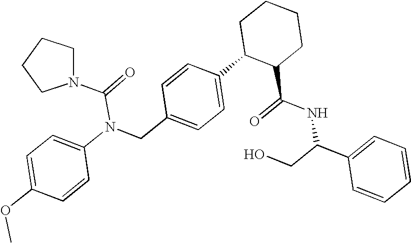 | 555.31 | 4.03 |
| 52 | 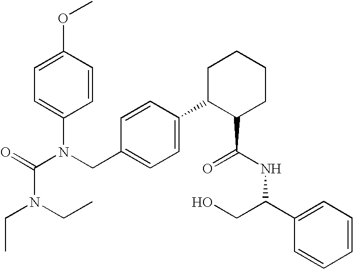 | 557.33 | 4.24 |
| 53 | 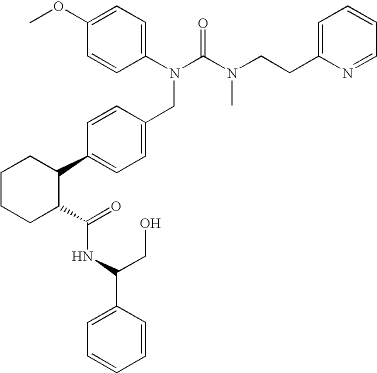 | 620.34 | 3.04 |
| 54 | 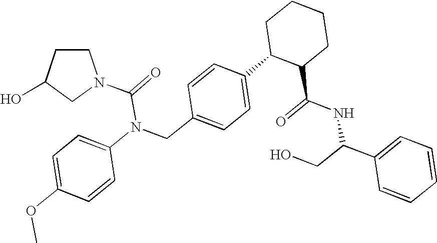 | 571.3 | 3.47 |
| 55 | 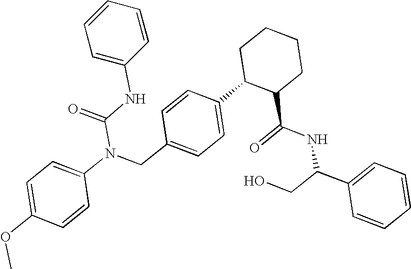 | 577.29 | 4.25 |
| 56 | 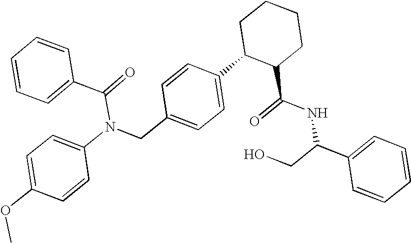 | 562.28 | 4.13 |
| 57 | 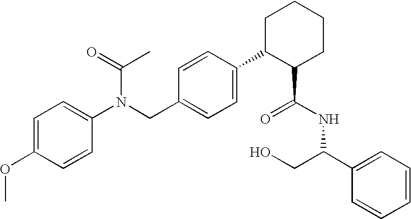 | 500.27 | 3.74 |
| 58 | 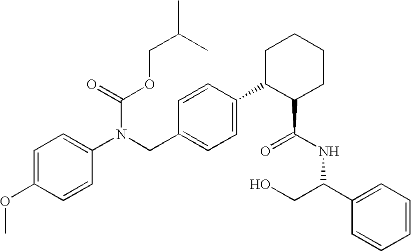 | 558.31 | 4.47 |
| 59 | 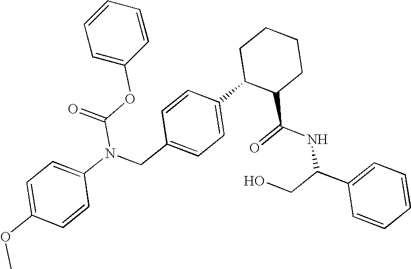 | 578.28 | 4.38 |
| 60 | 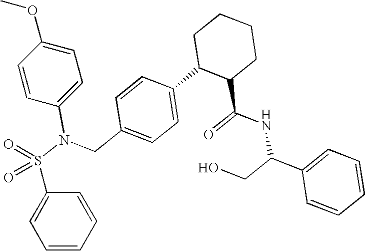 | 598.25 | 4.29 |
| 61 | 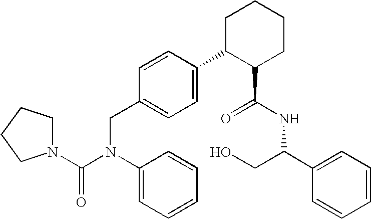 | 525.3 | 4.1 |
| 62 | 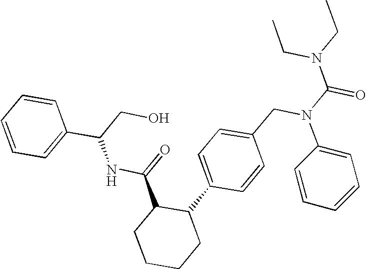 | 527.31 | 4.32 |
| 63 | 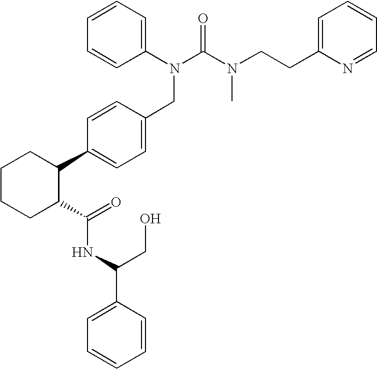 | 590.33 | 3.07 |
| 64 | 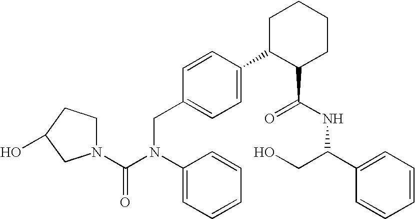 | 541.29 | 3.5 |
| 65 | 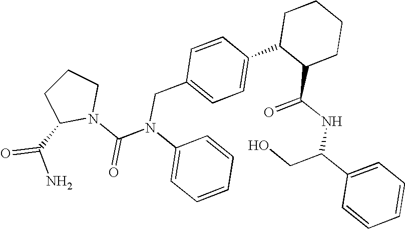 | 568.3 | 3.5 |
| 66 | 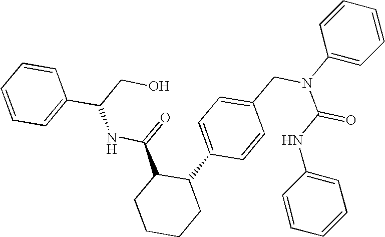 | 547.28 | 4.26 |
| 67 | 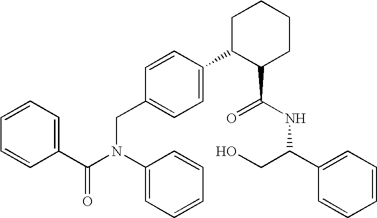 | 532.27 | 4.18 |
| 68 | 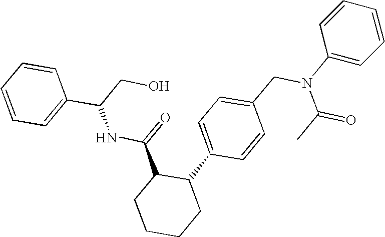 | 470.26 | 3.75 |
| 69 | 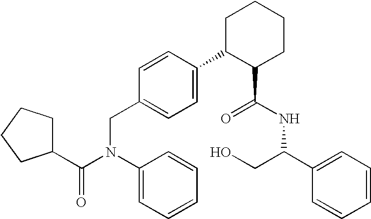 | 524.3 | 4.47 |
| 70 | 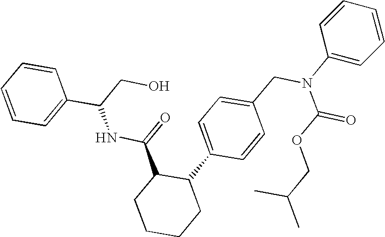 | 528.3 | 4.54 |
| 71 | 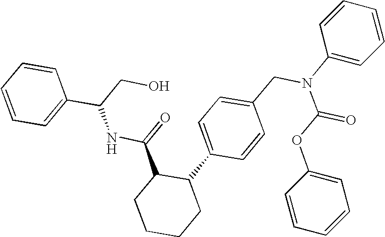 | 548.27 | 4.41 |
| 72 | 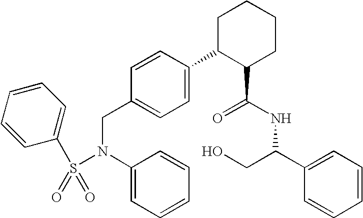 | 568.24 | 4.31 |
| 73 | 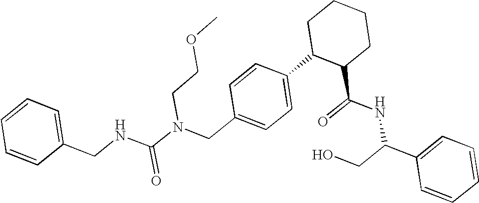 | 543.31 | 3.88 |
| 74 | 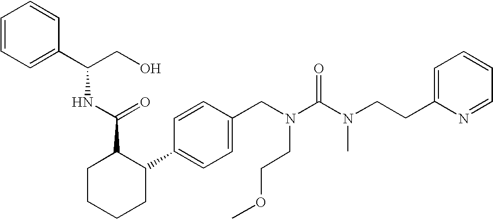 | 572.34 | 2.7 |
| 75 | 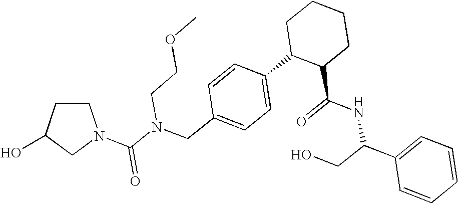 | 523.3 | 3.12 |
| 76 | 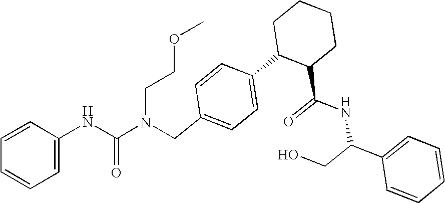 | 529.29 | 4.03 |
| 77 | 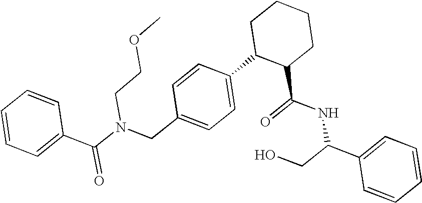 | 514.28 | 3.76 |
| 78 | 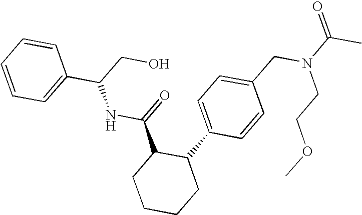 | 452.27 | 3.29 |
| 79 | 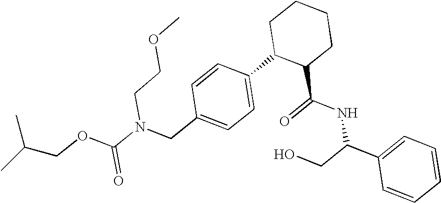 | 510.31 | 4.2 |
| 80 | 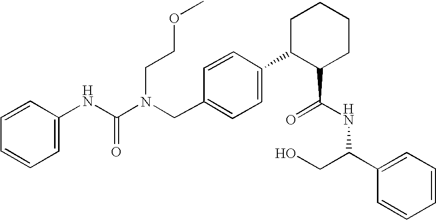 | 530.28 | 4.12 |
| 81 | 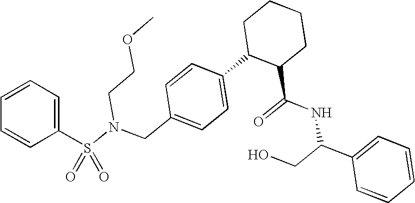 | 550.25 | 4.09 |
| 82 | 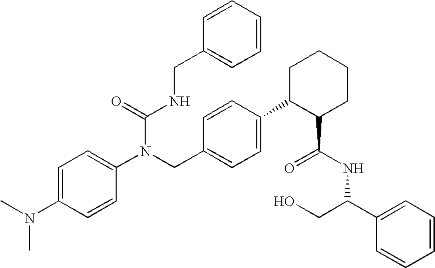 | 604.34 | 4.05 |
| 83 | 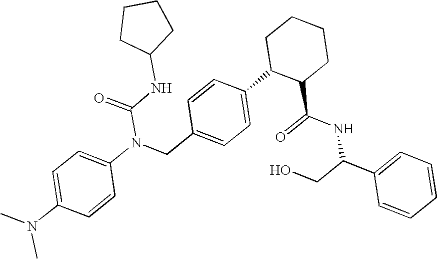 | 582.36 | 4.09 |
| 84 | 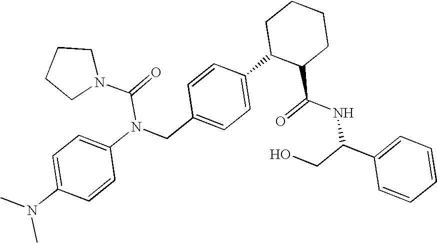 | 568.34 | 3.43 |
| 85 | 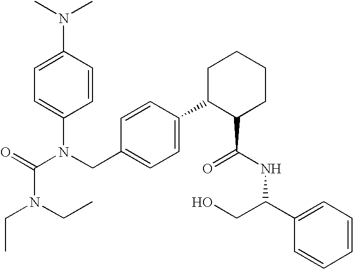 | 570.36 | 3.61 |
| 86 | 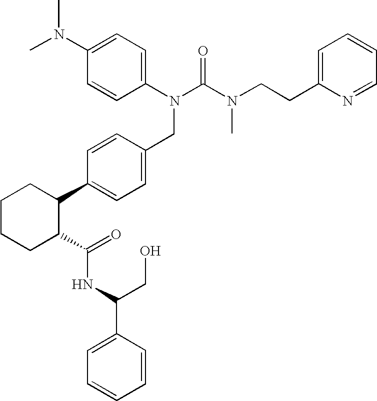 | 633.37 | 2.67 |
| 87 | 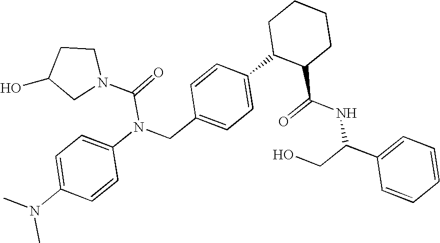 | 584.34 | 2.87 |
| 88 | 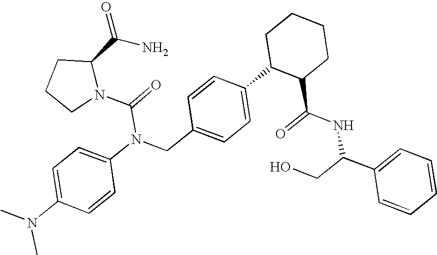 | 611.35 | 2.9 |
| 89 | 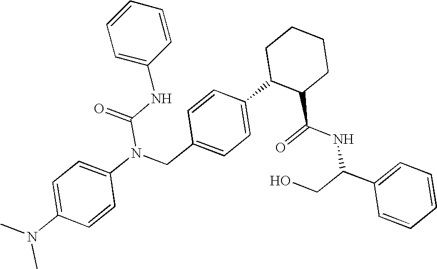 | 590.33 | 4.22 |
| 90 | 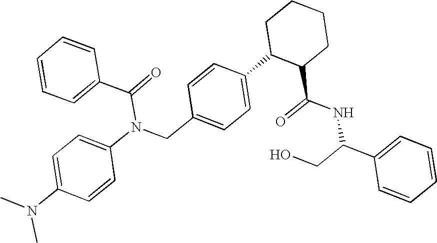 | 575.31 | 4.06 |
| 91 | 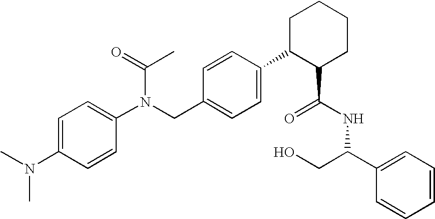 | 513.3 | 3.55 |
| 92 | 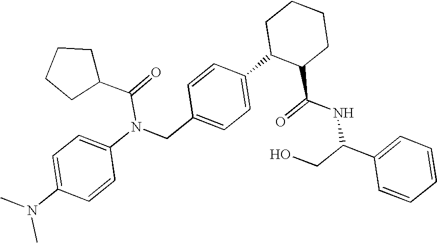 | 567.35 | 4.42 |
| 93 | 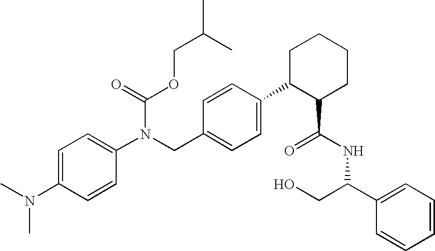 | 571.34 | 4.19 |
| 94 | 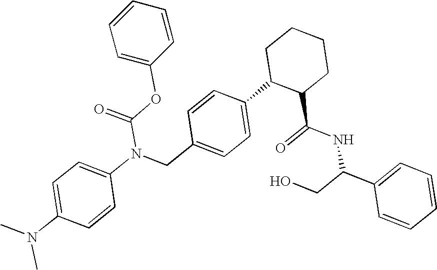 | 591.31 | 4.22 |
| 95 | 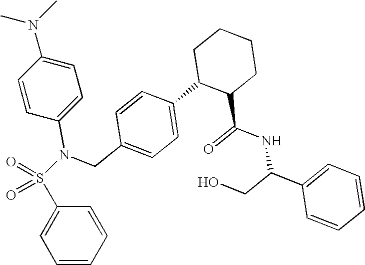 | 611.28 | 4.27 |
| 96 | 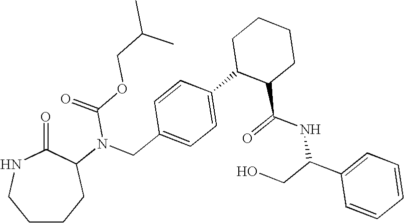 | 563.34 | 3.73 |
| 97 | 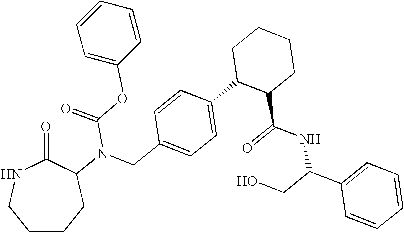 | 583.3 | 3.69 |
| 98 | 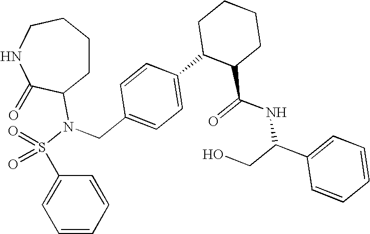 | 603.28 | 3.71 |
| 99 | 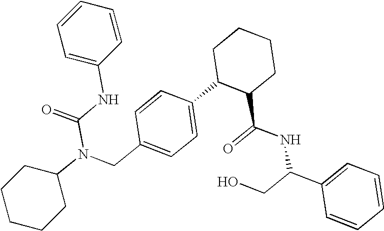 | 553.33 | 4.37 |
| 100 | 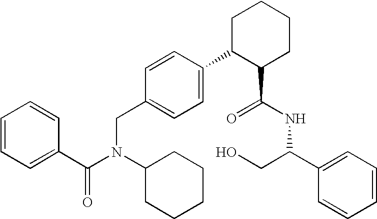 | 538.32 | 4.41 |
| 101 | 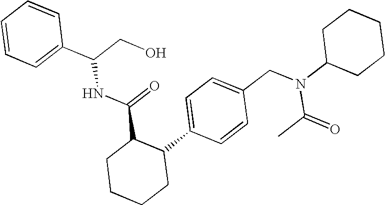 | 476.3 | 3.86 |
| 102 | 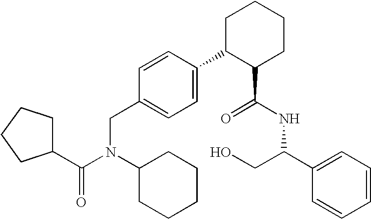 | 530.35 | 4.57 |
| 103 | 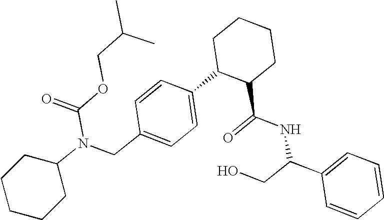 | 534.35 | 4.91 |
| 104 | 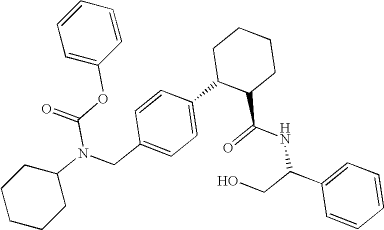 | 554.31 | 4.75 |
| 105 | 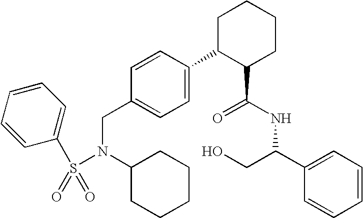 | 574.29 | 4.62 |
| 106 | 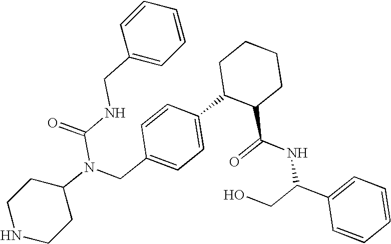 | 568.34 | 2.73 |
| 107 | 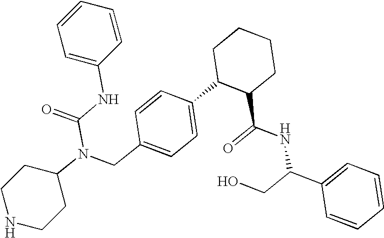 | 554.33 | 2.72 |
| 108 | 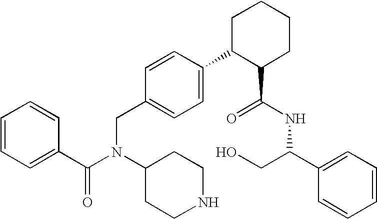 | 539.31 | 2.61 |
| 109 | 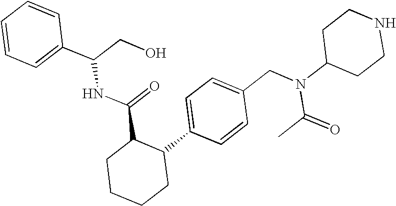 | 477.3 | 2.24 |
| 110 | 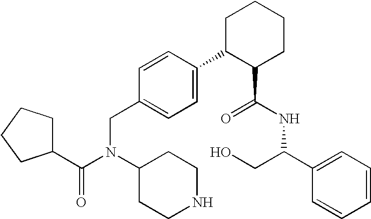 | 531.35 | 2.71 |
| 111 | 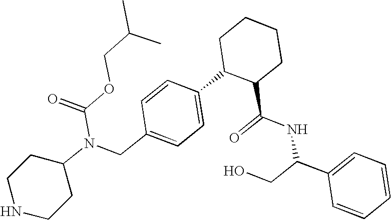 | 535.34 | 2.84 |
| 112 | 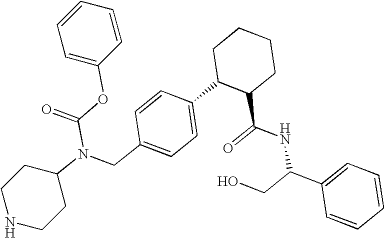 | 555.31 | 2.79 |
| 113 | 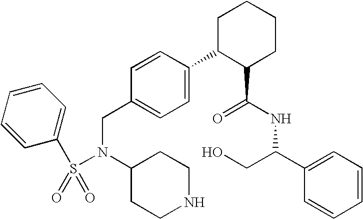 | 575.28 | 2.79 |

The compounds listed in the preceding table were characterized as follows:

Analytical Parameters

All products were characterized by LC-MS. The following separation system was routinely used for this: BP 1100 with UV detector (208-400 nm), 40° C. oven temperature, Waters Symmetry C18 column (50 mm×2.1 mm, 3.5 μm), mobile phase A: 99.9% acetonitrile/0.1% formic acid, mobile phase B: 99.9% water/0.1% formic acid; gradient:

| Time [min] | A: % | B: % | Flow rate [ml/min] |
|---|---|---|---|
| 0.00 | 10.0 | 90.0 | 0.50 |
| 4.00 | 90.0 | 10.0 | 0.50 |
| 6.00 | 90.0 | 10.0 | 0.50 |
| 6.10 | 10.0 | 90.0 | 1.00 |
| 7.50 | 10.0 | 90.0 | 0.50 |

The substances were detected using a Micromass Quattro LCZ MS, ionization: ESI positive/negative. The retention time is stated in minutes.

The compounds listed in the following table were furthermore prepared in an analogous way:

| | | | LC | Retention |
|---|---|---|---|---|
| Example | Structure | Mass | method | time [min] |
| 237 | 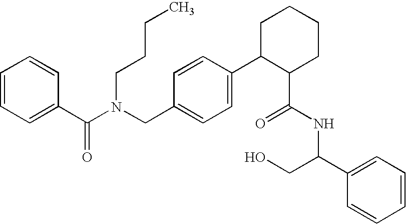 | 512.3 | Method 6 | 4.28 |
| 238 | 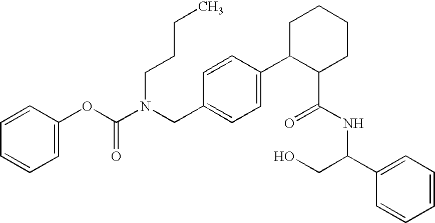 | 528.3 | Method 6 | 4.72 |
| 239 | 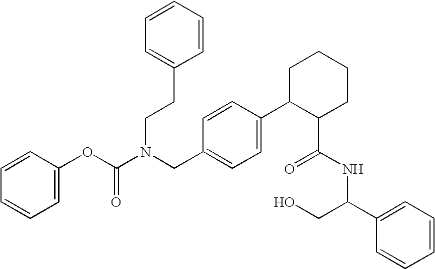 | 576.3 | Method 6 | 4.76 |
| 240 | 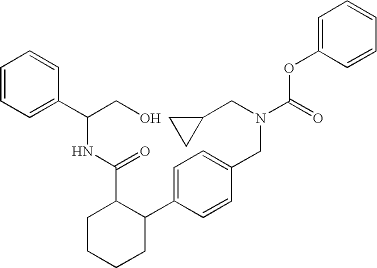 | 526.3 | Method 6 | 4.54 |
| 241 | 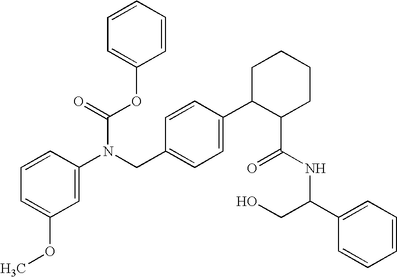 | 578.3 | Method 6 | 4.45 |
| 242 | 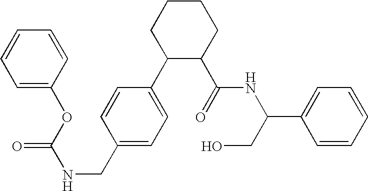 | 528.3 | Method 6 | 3.92 |
| 243 | 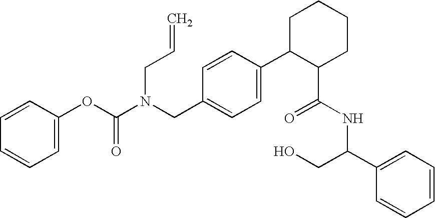 | 512.3 | Method 6 | 4.41 |
| 244 | 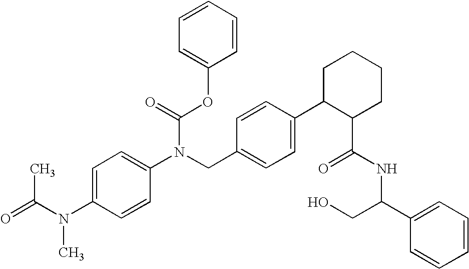 | 619.3 | Method 6 | 3.93 |
| 245 | 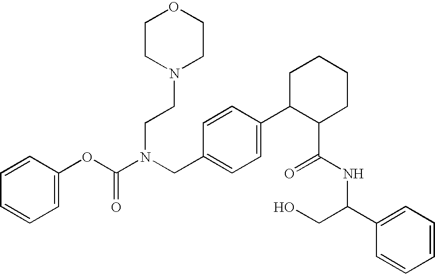 | 585.3 | Method 6 | 2.89 |
| 246 | 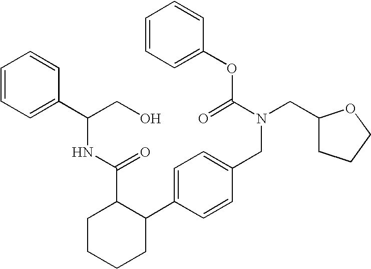 | 556.3 | Method 6 | 4.28 |
| 247 | 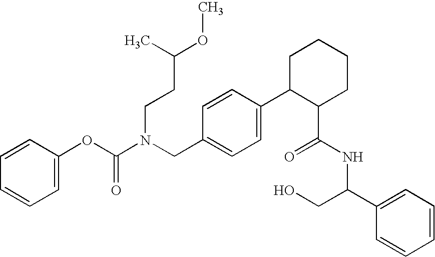 | 558.3 | Method 6 | 4.34 |
| 248 | 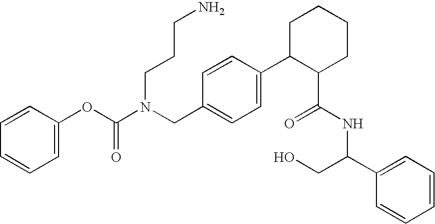 | 529.3 | Method 6 | 2.88 |
| 249 | 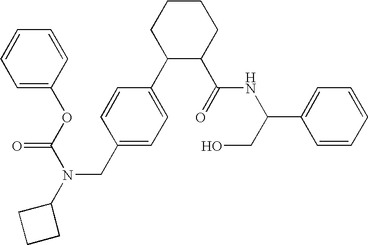 | 526.3 | Method 6 | 4.56 |
| 250 | 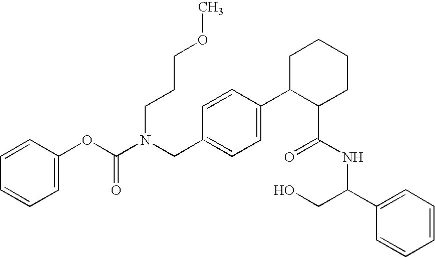 | 544.2 | Method 6 | 4.22 |
| 251 | 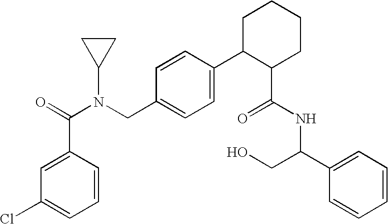 | 530.23 | Method 6 | 4.33 |
| 252 | 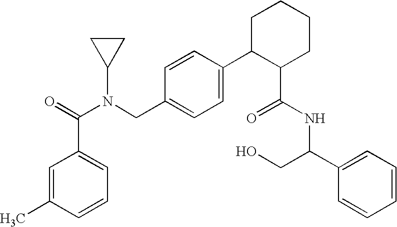 | 510.29 | Method 6 | 4.19 |
| 253 | 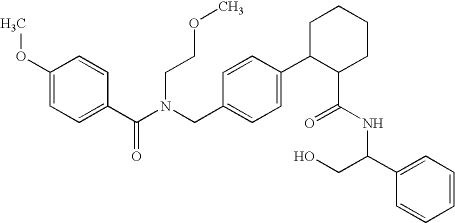 | 544.29 | Method 6 | 3.72 |
| 254 | 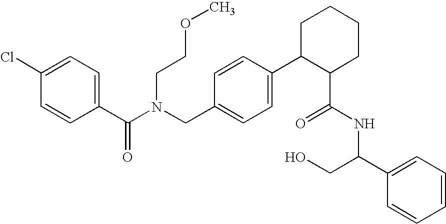 | 548.24 | Method 6 | 3.99 |
| 255 | 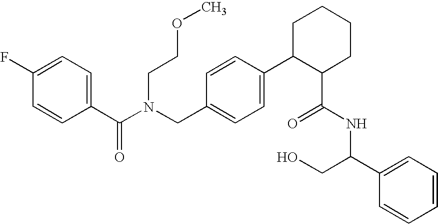 | 532.27 | Method 6 | 3.79 |
| 256 | 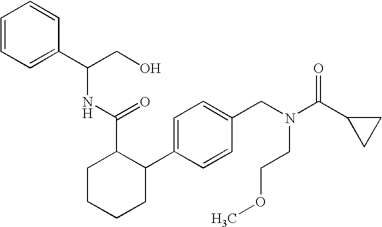 | 478.28 | Method 6 | 3.52 |
| 257 | 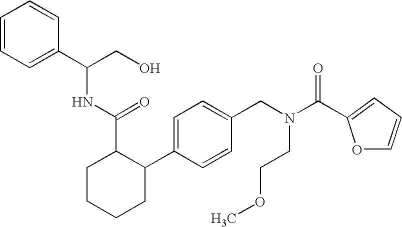 | 504.26 | Method 6 | 3.58 |
| 258 | 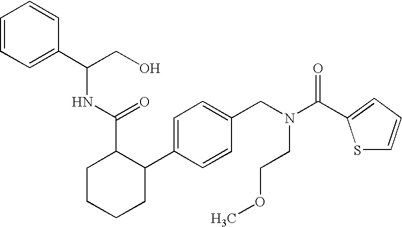 | 520.24 | Method 6 | 3.74 |
| 259 | 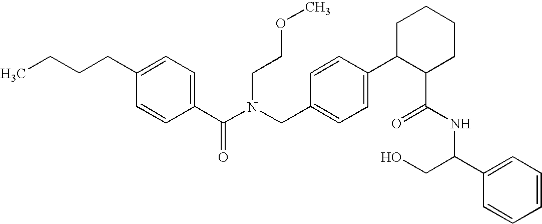 | 570.35 | Method 6 | 4.51 |
| 260 | 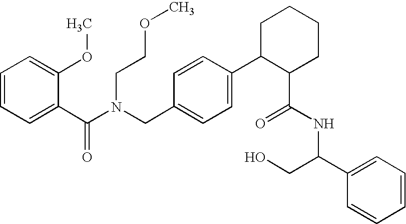 | 544.29 | Method 6 | 3.7 |
| 261 | 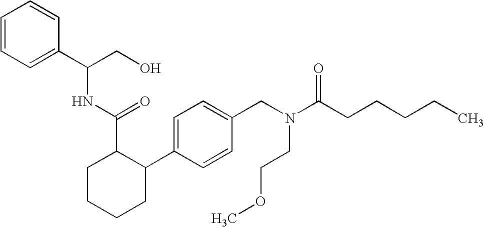 | 508.33 | Method 6 | 4.05 |
| 262 | 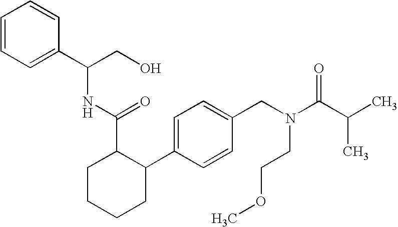 | 480.3 | Method 6 | 3.61 |
| 263 | 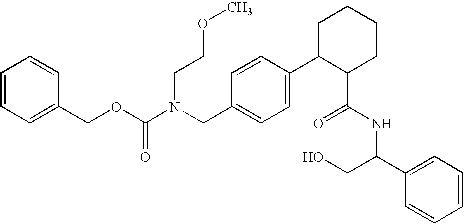 | 544.29 | Method 6 | 4.15 |
| 264 | 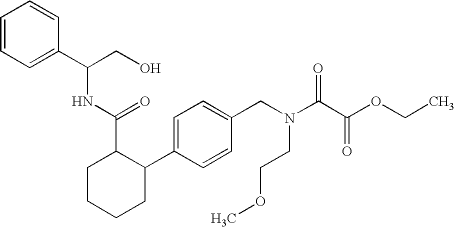 | 510.27 | Method 6 | 3.7 |
| 265 | 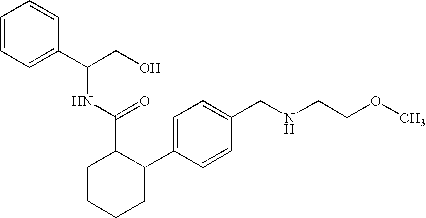 | 410.26 | Method 6 | 2.11 |
| 266 | 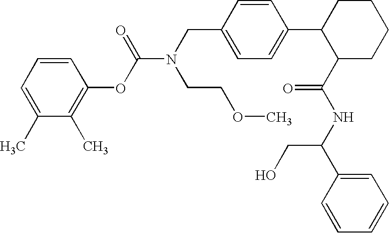 | 558.31 | Method 6 | 4.37 |
| 267 | 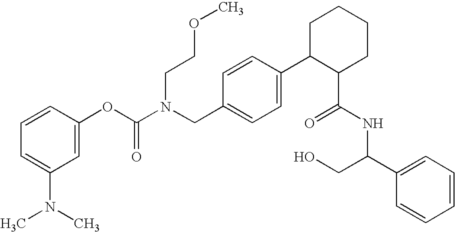 | 573.32 | Method 6 | 3.97 |
| 268 | 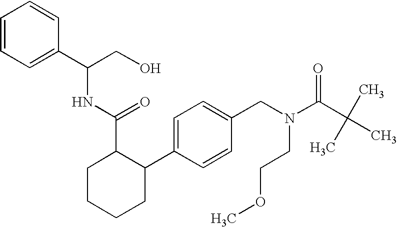 | 494.31 | Method 6 | 3.85 |
| 269 | 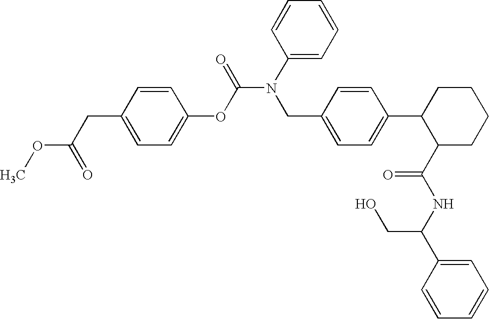 | 620.29 | Method 6 | 4.29 |
| 270 | 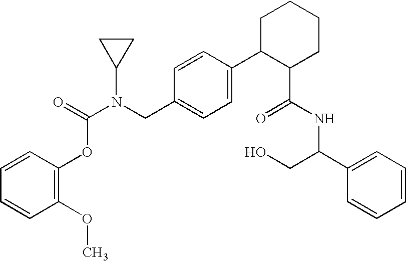 | 542.28 | Method 6 | 4.21 |
| 271 | 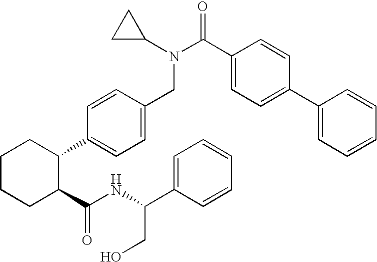 | 572.3 | Method 11 | 4.38 |
| 272 | 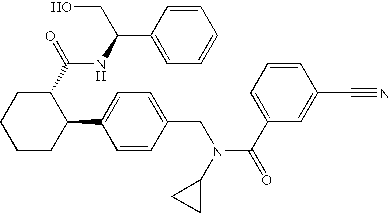 | 521.27 | Method 11 | 3.81 |
| 273 | 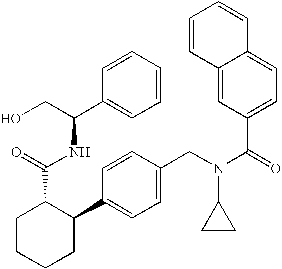 | 546.29 | Method 11 | 4.17 |
| 274 | 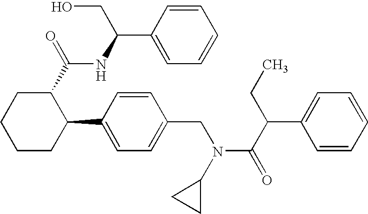 | 538.32 | Method 11 | 4.37 |
| 275 | 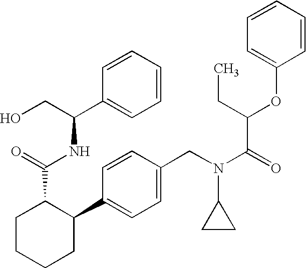 | 554.31 | Method 11 | 4.2 |
| 276 | 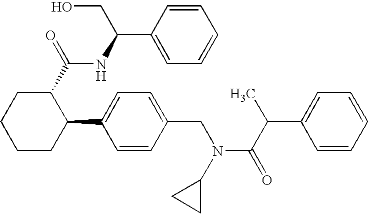 | 524.3 | Method 11 | 4.18 |
| 277 | 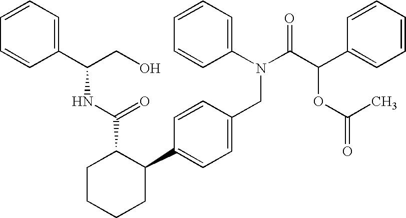 | 604.29 | Method 11 | 4.24 |
| 278 | 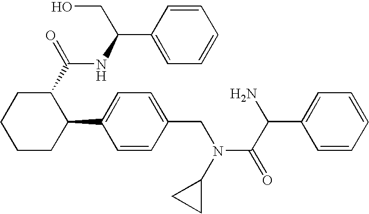 | 525.3 | Method 11 | 2.87 |
| 279 | 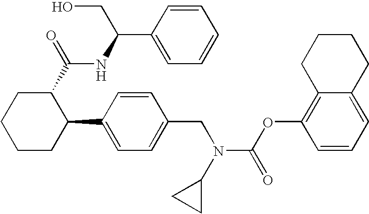 | 566.31 | Method 11 | 4.67 |
| 280 | 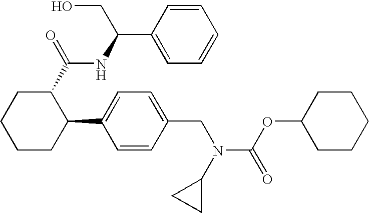 | 518.31 | Method 11 | 4.5 |
| 281 | 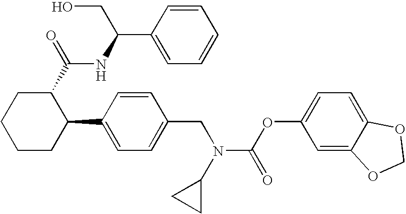 | 556.26 | Method 11 | 4.12 |
| 282 | 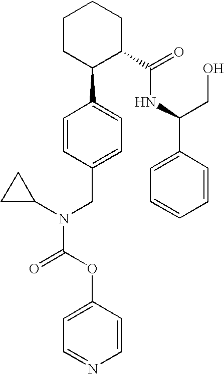 | 513.26 | Method 11 | 3.08 |
| 283 | 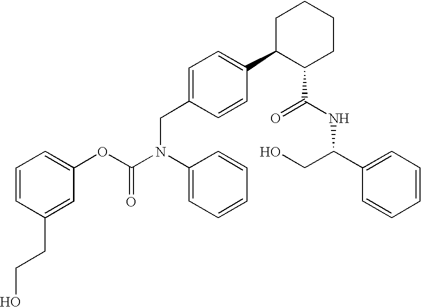 | 592.29 | Method 11 | 3.96 |
| 284 | 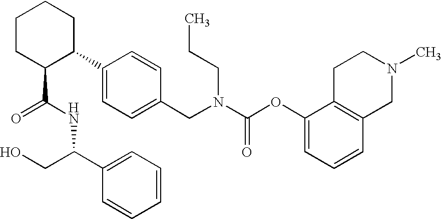 | 583.34 | Method 11 | 3 |
| 285 | 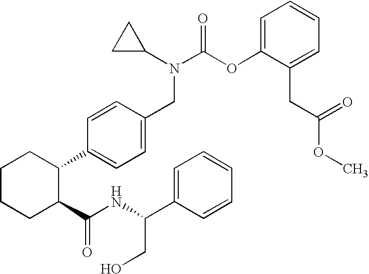 | 584.29 | Method 11 | 4.07 |
| 286 | 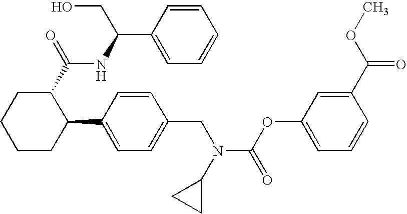 | 570.27 | Method 11 | 4.18 |
| 287 | 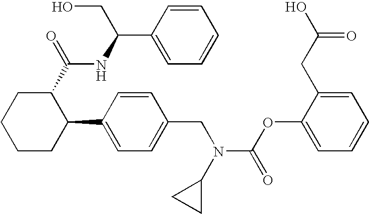 | 570.27 | Method 11 | 3.75 |
| 288 | 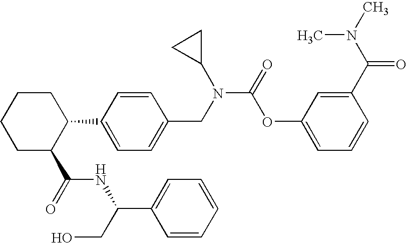 | 583.3 | Method 11 | 3.72 |

1. A compound of the formula (I): 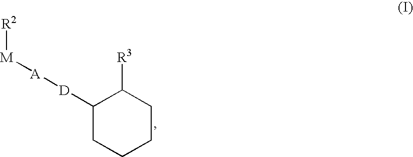
in which
M is a group —N(—R¹)— or an oxygen atom —O—,
A is a group —C(═O)— or —CH₂— or a chemical bond,
D is 5- or 6-membered heteroarylene with up to three heteroatoms chosen from N, O and S, or phenylene, each of which may be substituted up to three times, independently of one another, by halogen, hydroxyl, cyano, carboxyl, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,
R¹ is hydrogen, benzyl, (C₂-C₆)-alkenyl, (C₁-C₆)-alkyl, optionally benzo-fused (C₃-C₈)-cycloalkyl,
where alkyl and cycloalkyl in turn may be substituted up to three times, independently of one another, by hydroxyl, amino, (C₁-C₆)-alkoxy, phenyl, 5- or 6-membered heterocyclyl with up to three heteroatoms chosen from N, O and S, (C₃-C₈)-cycloalkyl or mono- or di-(C₁-C₆)-alkylamino,
(C₆-C₁₀)-aryl, or 5- to 10-membered heteroaryl with up to three heteroatoms chosen from N, O and S, or 5- or 6-membered heterocyclyl with up to three heteroatoms chosen from N, O and S,
where aryl, heteroaryl and heterocyclyl in turn may be substituted up to three times, independently of one another, by halogen, hydroxyl, oxo, cyano, nitro, trifluoromethyl, trifluoromethoxy, (C₁-C₆)-alkyl, (C₁-C₆)-alkoxy, (C₁-C₆)-alkoxycarbonyl, N-acetyl, N-methylamino or mono- or di-(C₁-C₆)-alkylamino,
R² is hydrogen, (C₁-C₆)-alkyl, (C₃-C₈)-cycloalkyl,
where alkyl and cycloalkyl in turn may be substituted up to three times, independently of one another, by hydroxyl, (C₁-C₆)-alkoxy, mono- or di-(C₁-C₆)-alkylamino, optionally halogen-, trifluoromethyl- or (C₁-C₆)-alkoxy-substituted phenyl, biphenyl, naphtyl, optionally halogen-substituted 5- or 6-membered heteroaryl with up to three heteroatoms chosen from N, O and S, or optionally hydroxyl-substituted 5- to 10-membered heterocyclyl with up to three heteroatoms chosen from N, O and S,
(C₆-C₁₀)-aryl, 5- to 10-membered heteroaryl with up to three heteroatoms chosen from N, O and S, or 5- or 6-membered heterocyclyl with up to three heteroatoms chosen from N, O and S,
where aryl, heteroaryl and heterocyclyl in turn may be substituted up to three times, independently of one another, by phenyl, benzyl, morpholinyl, halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino, or a radical of the formula —C(═O)—R⁴ or —SO₂—R⁴,
in which
R⁴ is hydrogen, (C₁-C₆)-alkyl,
which may in turn be substituted by hydroxyl, amino, phenyl, (C₆-C₁₀)-aryloxy, (C₁-C₆)-alkanoyloxy or (C₁-C₄)-alkoxy,
(C₆-C₁₀)-aryl, 5- to 10-membered heteroaryl with up to three heteroatoms chosen from N, O and S, 5- to 10-membered heterocyclyl with up to three heteroatoms chosen from N, O and S,
in which aryl, heteroaryl and heterocyclyl in turn may be substituted up to twice, independently of one another, by halogen, optionally hydroxyl-substituted (C₁-C₆)-alkyl, (C₁-C₆)-alkoxy, (C₁-C₆)-alkoxycarbonyl, phenyl or cyano,
(C₃-C₈)-cycloalkyl, (C₁-C₆)-alkoxycarbonyl or a radical of the formula —NR⁵R⁶ or —OR⁷,
in which
R⁵ and R⁶ are, independently of one another, hydrogen, (C₆-C₁₀)-aryl, adamantyl, (C₁-C₈)-alkyl,
whose chain may be interrupted by one or two oxygen atoms and which may be substituted up to three times by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms chosen from N, O and S, or by 5- to 10-membered heteroaryl with up to three heteroatoms chosen from N, O and S,
(C₃-C₈)-cycloalkyl,
which may be substituted up to three times by (C₁-C₄)-alkyl, hydroxyl or oxo,
or 5- or 6-membered heterocyclyl with up to two heteroatoms chosen from N, O and S, where N is substituted by hydrogen or (C₁-C₄)-alkyl,
or
R⁵ and R⁶ together with the nitrogen atom to which they are bonded form a 4- to 7-membered saturated heterocycle in which up to two ring carbon atoms are replaced by heteroatoms chosen from N, O and S, and which may be substituted by hydroxyl, oxo, aminocarbonyl, (C₁-C₆)-alkyl or (C₁-C₆)-alkoxy-(C₁-C₆)-alkyl,
and
R⁷ is 5- or 6-membered heteroaryl with up to three heteroatoms chosen from N, O and S,
which may be substituted up to twice, independently of one another, by (C₁-C₆)-alkyl, (C₁-C₆)-alkylthio or oxo, (C₆-C₁₀)-aryl,
which may be substituted up to twice, independently of one another, by optionally (C₁-C₆)-alkoxycarbonyl- or carboxyl-subsituted (C₁-C₆)-alkyl, (C₁-C₆)-alkoxy, di-(C₁-C₆)-alkylaminocarbonyl, mono- or di-(C₁-C₆)-alkylamino,
adamantyl, tetrahydronaphtyl, (C₁-C₈)-alkyl,
whose chain may be interrupted by one or two oxygen atoms and which may be substituted up to three times, independently of one another, by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms chosen from N, O and S or by 5- to 10-membered heteroaryl with up to three heteroatoms chosen from N, O and S, (C₃-C₈)-cycloalkyl,
which may be substituted up to three times, independently of one another, by (C₁-C₄)-alkyl, hydroxyl or oxo,
or 5- to 10-membered heterocyclyl with up to two heteroatoms chosen from N, O and S, where N is substituted by hydrogen or (C₁-C₄)-alkyl,
or
R¹ and R² together with the nitrogen atom to which they are bonded form a 5- to 10-membered saturated heterocycle with up to two further heteroatoms chosen from N, O and S, which is optionally substituted up to twice, independently of one another, by benzyl or (C₆-C₁₀)-aryl which in turn may be substituted by halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,
R³ is a group 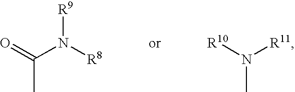
in which
R⁸ is a group of the formula 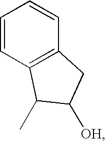
is (C₃-C₈)-cycloalkyl which may be substituted by (C₁-C₈)-alkyl, (C₆-C₁₀)-aryl, 5- to 10-membered heterocyclyl with up to three heteroatoms chosen from N, O and S or 5- to 10-membered heteroaryl with up to three heteroatoms chosen from N, O and S,
where aryl, heterocyclyl and heteroaryl in turn may be substituted up to three times, independently of one another, by halogen, trifluoromethyl, cyano, nitro, hydroxyl, (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl or carboxyl,
or
is a methyl group,
which may be substituted up to three times, independently of one another, by hydrogen, trifluoromethyl, (C₃-C₈)-cycloalkyl, (C₁-C₈)-alkyl, whose chain may be interrupted by a sulfur atom or an S(O) or SO₂ group and which may be substituted up to twice, independently of one another, by hydroxyl, (C₁-C₆)-alkoxy, (C₁-C₆)-alkoxycarbonyl, halogen, cyano, nitro, trifluoromethoxy, oxo, amino, mono- or di-(C₁-C₆)-alkylamino, 5- or 6-membered heterocyclyl with up to three heteroatoms chosen from N, O and S, or carboxamide,
(C₁-C₆)-alkoxycarbonyl, (C₆-C₁₀)-aryl, benzyl, 5- to 10-membered heterocyclyl with up to three heteroatoms chosen from N, O and S, or 5- to 10-membered heteroaryl with up to three heteroatoms chosen from N, O and S,
where aryl, benzyl, heterocyclyl and heteroaryl may be substituted up to three times, independently of one another, by halogen, trifluoromethyl, cyano, nitro, hydroxyl, optionally hydroxyl-substituted (C₁-C₆)-alkyl, (C₃-C₈)-cycloalkyl, (C₁-C₆)-alkoxy, amino, mono- or di-(C₁-C₆)-alkylamino, (C₁-C₆)-alkoxycarbonyl, carboxyl, (C₁-C₆)-alkylcarbonylamino, (C₁-C₆)-alkoxycarbonylamino, aminocarbonyl, mono- or di-(C₁-C₆)-alkylaminocarbonyl which in turn may be substituted by (C₁-C₆)-alkoxy, or amidosulfone, mono- or di-(C₁-C₆)-alkylamidosulfone which in turn may be substituted by (C₁-C₆)-alkoxy,
R⁹ is hydrogen, (C₁-C₆)-alkoxy, (C₁-C₆)-alkyl- and/or phenyl-substituted amino, (C₁-C₆)-alkyl, (C₃-C₈)-cycloalkyl,
where alkyl and cycloalkyl in turn may be substituted up to three times, independently of one another, by hydroxyl or mono- or di-(C₁-C₆)-alkylamino,
(C₆-C₁₀)-aryl, or 5- to 10-membered heteroaryl with up to three heteroatoms chosen from N, O and S, or 5- or 6-membered heterocyclyl with up to three heteroatoms chosen from N, O and S,
where aryl, heteroaryl and heterocyclyl in turn may be substituted up to three times, independently of one another, by halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,
or
R⁸ and R⁹ together with the nitrogen atom to which they are bonded form a 5- to 10-membered, optionally bicyclic heterocycle in which up to two ring carbon atoms are replaced by heteroatoms chosen from N, O and S and which may be substituted up to four times, independently of one another, by hydroxyl, (C₁-C₆)-alkyl, (C₁-C₆)-alkoxy, hydroxy-(C₁-C₆)-alkyl, (C₁-C₆)-alkoxy-(C₁-C₆)-alkyl, oxo, amino or mono- or di-(C₁-C₆)-alkylamino,
R¹⁰ is hydrogen, (C₁-C₆)-alkoxy, (C₁-C₆)-alkyl, (C₃-C₈)-cycloalkyl, where alkyl and cycloalkyl in turn may be substituted, independently of one another, up to three times by hydroxyl or mono- or di-(C₁-C₆)-alkylamino,
(C₆-C₁₀)-aryl, or 5- to 10-membered heteroaryl with up to three heteroatoms chosen from N, O and S or 5- or 6-membered heterocyclyl with up to three heteroatoms chosen from N, O and S,
where aryl, heteroaryl and heterocyclyl may in turn be substituted, independently of one another, up to three times by halogen, hydroxyl, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,
R¹¹ is a radical of the formula —C(═O)—R¹² or —SO₂—R¹²,
in which
R¹² is hydrogen, (C₁-C₆)-alkyl which may in turn be substituted by hydroxyl or (C₁-C₄)-alkoxy, or (C₆-C₁₀)-aryl, or 5- to 10-membered heteroaryl with up to three heteroatoms chosen from N, O and S, in which aryl and heteroaryl in turn may be substituted, independently of one another, by halogen, or (C₃-C₈)-cycloalkyl or a radical of the formula —NR¹³R¹⁴ or —OR¹⁵,
in which
R¹³ and R¹⁴ are, independently of one another, hydrogen, (C₆-C₁₀)-aryl, adamantyl, (C₁-C₈)-alkyl,
whose chain may be interrupted by one or two oxygen atoms and which may be substituted up to three times by hydroxyl, optionally halogen-, (C₁-C₆)-alkoxy- or amino-substituted phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms chosen from N, O and S or by 5- to 10-membered heteroaryl with up to three heteroatoms chosen from N, O and S,
(C₃-C₈)-cycloalkyl,
which may be substituted up to three times by (C₁-C₄)-alkyl, hydroxyl or oxo,
or 5- or 6-membered heterocyclyl with up to two heteroatoms chosen from N, O and S, where N is substituted by hydrogen or (C₁-C₄)-alkyl,
or
R¹³ and R¹⁴ together with the nitrogen atom to which they are bonded form a 4- to 7-membered saturated heterocycle which may contain up to two further heteroatoms chosen from N, O and S and is optionally substituted by hydroxyl, oxo, (C₁-C₆)-alkyl or (C₁-C₆)-alkoxy-(C₁-C₆)-alkyl,
R¹⁵ is (C₆-C₁₀)-aryl, adamantyl, (C₁-C₈)-alkyl,
whose chain may be interrupted by one or two oxygen atoms and which may be substituted up to three times, independently of one another, by hydroxyl, phenyl, trifluoromethyl, (C₃-C₈)-cycloalkyl, (C₁-C₆)-alkoxy, mono- or di-(C₁-C₆)-alkylamino, 5- or 6-membered heterocyclyl with up to three heteroatoms chosen from N, O and S, or by 5- to 10-membered heteroaryl with up to three heteroatoms chosen from N, O and S, (C₃-C₈)-cycloalkyl,
which may be substituted up to three times, independently of one another, by (C₁-C₄)-alkyl, hydroxyl or oxo,
or 5- or 6-membered heterocyclyl with up to two heteroatoms chosen from N, O and S, where N is substituted by hydrogen or (C₁-C₄)-alkyl,
or a salt, hydrate, hydrate of the salt or solvate thereof.

2. A compound of the formula (I) as defined in claim 1,
in which
M is a group —N(—R¹)— or an oxygen atom —O—,
A is a group —C(═O)— or —CH₂— or a chemical bond,
D is 5- or 6-membered heteroarylene with up to three heteroatoms chosen from N, O and S, or phenylene, each of which may be substituted up to twice, independently of one another, by halogen, hydroxyl, cyano, carboxyl, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,
R¹ is hydrogen, benzyl (C₂-C₆)-alkenyl, (C₁-C₆)-alkyl,
where alkyl in turn may be substituted by (C₁-C₄)-alkoxy, phenyl, (C₃-C₈)-cycloalkyl or mono- or di-(C₁-C₄)-alkylamino,
phenyl or 5- or 6-membered heteroaryl with up to three heteroatoms chosen from N, O and S,
where phenyl and heteroaryl in turn may be substituted up to twice, independently of one another, by halogen, trifluoromethyl, trifluoromethoxy, (C₁-C₄)-alkyl, (C₁-C₄)-alkoxy, (C₁-C₄)-alkoxycarbonyl, N-acetyl,N-methylamino or mono- or di-(C₁-C₄)-alkylamino,
R² is (C₁-C₆)-alkyl, (C₃-C₈)-cycloalkyl,
where alkyl and cycloalkyl in turn may be substituted up to twice, independently of one another, by (C₁-C₄)-alkoxy, mono- or di-(C₁-C₆)-alkylamino, optionally halogen-, trifluoromethyl- or (C₁-C₆)-alkoxy-substituted phenyl, biphenyl, naphtyl or optionally halogen-substituted 5- or 6-membered heteroaryl with up to three heteroatoms chosen from N, O and S,
phenyl or 5- or 6-membered heteroaryl with up to three heteroatoms chosen from N, O and S,
where aryl and heteroaryl in turn may be substituted up to twice, independently of one another, by phenyl, benzyl, morpholinyl, halogen, cyano, nitro, trifluoromethyl, trifluoromethoxy, (C₁-C₆)-alkyl, (C₁-C₆)-alkoxy, (C₁-C₆)-alkoxycarbonyl or mono- or di-(C₁-C₆)-alkylamino,
or a radical of the formula —C(═O)—R⁴ or —SO₂—R⁴,
in which
R⁴ is hydrogen, methyl, or ethyl,
each of which in turn may be substituted by hydroxyl, amino, phenyl, (C₆-C₁₀)-aryloxy, (C₁-C₆)-alkanoyloxy or (C₁-C₄)-alkoxy,
phenyl, 5- or 6-membered heteroaryl with up to two heteroatoms chosen from N, O and S, 5- or 6-membered heterocyclyl with up to two heteroatoms chosen from N, O and S,
in which phenyl, heteroaryl and heterocyclyl may in turn be substituted up to twice, independently of one another, by halogen, optionally hydroxyl-substituted (C₁-C₄)-alkyl, (C₁-C₄)-alkoxy, (C₁-C₄)-alkoxycarbonyl, phenyl or cyano,
(C₃-C₈)-cycloalkyl or a radical of the formula —NR⁵R or —OR⁷,
in which
R⁵ and R⁶ are, independently of one another, phenyl or (C₁-C₆)-alkyl, whose chain may be interrupted by an oxygen atom and which may be substituted up to twice by phenyl, trifluoromethyl, (C₃-C₆)-cycloalkyl or (C₁-C₆)-alkoxy,
or
R⁵ and R⁶ together with the nitrogen atom to which they are bonded form a 5- to 7-membered saturated heterocycle in which one ring carbon atom is replaced by a heteroatom chosen from N, O of and S and which may be substituted by hydroxyl, oxo, (C₁-C₆)-alkyl or (C₁-C₂)-alkoxy-(C₁-C₂)-alkyl,
and
R⁷ is 5- or 6-membered heteroaryl with up to three heteroatoms chosen from N, O and S,
which may be substituted up to twice, independently of one another, by (C₁-C₆)-alkyl, (C₁-C₆)-alkylthio or oxo, (C₆-C₁₀)-aryl,
which may be substituted up to twice, independently of one another, by optionally (C₁-C₆)-alkoxy, di-(C₁-C₆)-alkylaminocarbonyl, mono- or di-(C₁-C₆)-alkylamino, tetrahydronaphtyl, (C₁-C₄)-alkyl,
whose chain may be interrupted by an oxygen atom and which may be substituted up to twice, independently of one another, by phenyl, trifluoromethyl, (C₃-C₆)-cycloalkyl or (C₁-C₆)-alkoxy, (C₃-C₈)-cycloalkyl,
which may be substituted up to three times, independently of one another, by (C₁-C₄)-alkyl, hydroxyl or oxo,
or 5- or 6-membered heterocyclyl with up to two heteroatomrs chosen from N, O and S, where N is substituted by hydrogen or (C₁-C₄)-alkyl,
or
R¹ and R² together with the nitrogen atom to which they are bonded form a 5- or 6-membered saturated heterocycle with up to one further heteroatom chosen from N, O and S, which is optionally substituted up to twice, independently of one another, by benzyl or phenyl which in turn may be substituted by halogen, hydroxyl, cyano, nitro, trifluoromethyl, trifluoromethoxy, (C₁-C₄)-alkyl, (C₁-C₄)-alkoxy, (C₁-C₄)-alkoxycarbonyl or mono- or di-(C₁-C₆)-methylamino,
R³ is a group 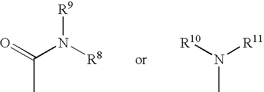
in which
R⁸ is a group of the formula 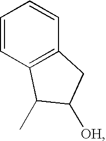
is (C₃-C₅)-cycloalkyl which may be substituted by phenyl or 5- or 6-membered heteroaryl with up to two heteroatoms chosen from N, O and S,
where phenyl and heteroaryl in turn may be substituted up to twice, independently of one another, by halogen, trifluoromethyl, cyano, nitro, hydroxyl, (C₁-C₆)-alkyl, (C₃-C₆)-cycloalkyl, (C₁-C₆)-alkoxy, amino, mono- or di-(C₁-C₂)-alkylamino,
or
is a methyl group,
which is substituted by hydrogen,
by trifluoromethyl, (C₃-C₆)-cycloalkyl or (C₁-C₄)-alkyl which may in turn be substituted by hydroxyl, (C₁-C₄)-alkoxy, halogen, cyano, trifluoromethoxy, amino, mono- or di-(C₁-C₄)-alkylamino, 5- or 6-membered heterocyclyl with up to two heteroatoms chosen from N, O and S, or carboxamide,
and by (C₆-C₁₀)-aryl or 5- to 10-membered heteroaryl with up to three heteroatoms chosen from N, O and S,
where aryl and heteroaryl in turn may be substituted up to twice, independently of one another, by halogen, trifluoromethyl, cyano, nitro, hydroxyl, optionally hydroxyl-substituted (C₁-C₄)-alkyl, (C₃-C₆)-cycloalkyl, (C₁-C₄)-alkoxy, amino, mono- or di-(C₁-C₄)-alkylamino, (C₁-C₄)-alkoxycarbonyl, carboxyl, (C₁-C₄)-alkylcarbonylamino, (C₁-C₄)-alkoxycarbonylamino, aminocarbonyl, mono- or di-(C₁-C₄)-alkylaminocarbonyl, amidosulfone, mono- or di-(C₁-C₄)-alkylamidosulfone,
R⁹ is hydrogen or (C₁-C₂)-alkyl- and phenyl-substituted amino,
R¹⁰ is hydrogen,
R¹¹ is a radical of the formula —C(═O)—R¹²,
in which
R¹² is a radical of the formula —NR¹³R¹⁴,
in which
R¹³ is hydrogen,
R¹⁴ is a methyl group which is substituted by hydrogen, methyl or ethyl and by phenyl which in turn may be substituted by halogen, (C₁-C₄)-alkoxy or amino,
or a salt, hydrate, hydrate of the salt or solvate thereof.

3. A compound of the formula (I) as claimed in claim 1,
in which
M is a group —N(—R¹)— or an oxygen atom —O—,
A is a group —CH₂— or a chemical bond,
D is 5- or 6-membered heteroarylene with up to two heteroatoms chosen from N, O and S, or phenylene, each of which may be substituted up to twice, independently of one another, by halogen, trifluoromethyl, trifluoromethoxy, (C₁-C₄)-alkyl or (C₁-C₄)-alkoxy,
R¹ is hydrogen, phenyl, (C₂-C₄)-alkenyl or (C₁-C₄)-alkyl,
where alkyl in turn may be substituted by methoxy, (C₃-C₆)-cycloalkyl or mono- or dimethylamino,
R² is (C₁-C₄)-alkyl, (C₃-C₆)-cycloalkyl,
where alkyl and cycloalkyl in turn may be substituted up to twice, independently of one another, by methoxy, mono- or dimethylamino, optionally halogen-, trifluoromethyl- or methoxy-substituted phenyl, biphenyl, naphtyl or optionally halogen-substituted 5- or 6-membered heteroaryl with up to two heteroatoms chosen from N, O and S,
phenyl or 5- or 6-membered heteroaryl with up to two heteroatoms chosen from N, O and S,
where aryl and heteroaryl in turn may be substituted up to twice, independently of one another, by halogen, cyano, nitro, trifluoromethyl, trifluoromethoxy, (C₁-C₄)-alkyl, (C₁-C₄)-alkoxy or mono- or dimethylamino,
or a radical of the formula —C(═O)—R⁴,
in which
R⁴ is methyl,
which may in turn be substituted by phenyl, phenyloxy or (C₁-C₂)-alkoxy, phenyl, 5- or 6-membered heteroaryl with up to two heteroatoms chosen from N, O and S, 5- or 6-membered heterocyclyl with up to two heteroatoms chosen from N, O and S,
in which phenyl, heteroaryl and heterocyclyl in turn may be substituted up to twice, independently of one another, by halogen, methoxy or (C₁-C₄)-alkoxycarbonyl,
(C₃-C₄)-cycloalkyl or a radical of the formula —OR⁷,
in which
R⁷ is 5- or 6-membered heteroaryl with up to two heteroatoms chosen from N, O and S,
which may be substituted up to twice, independently of one another, by (C₁-C₄)-alkyl or methylthio, phenyl,
which may be substituted up to twice, independently of one another, by optionally (C₁-C₄)-alkoxy, dimethylaminocarbonyl or mono- or dimethylamino, or tetrahydronaphtyl,
R³ is a group 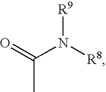
in which
R⁸ is (C₃-C₅)-cycloalkyl which may be substituted by phenyl or 5- or 6-membered heteroaryl with up to two heteroatoms chosen from N, O and S,
where phenyl and heteroaryl in turn may be substituted up to twice, independently of one another, by (C₁-C₄)-alkyl or (C₁-C₄)-alkoxy,
or
is a methyl group,
which is substituted by hydrogen,
by (C₁-C₃)-alkyl which may in turn be substituted by hydroxyl, (C₁-C₂)-alkoxy, amino or mono- or di-(C₁-C₄)-alkylamino,
and by phenyl or 5- to 10-membered heteroaryl with up to three heteroatoms chosen from N, O and S,
where phenyl and heteroaryl in turn may be substituted up to twice, independently of one another, by halogen, trifluoromethyl, cyano, nitro, hydroxyl, optionally hydroxyl-substituted (C₁-C₂)-alkyl, (C₃-C₆)-cycloalkyl, (C₁-C₄)-alkoxy, amino, mono- or di-(C₁-C₄)-alkylamino, (C₁-C₄)-alkoxycarbonyl, (C₁-C₄)-alkylcarbonylamino, (C₁-C₄)-alkoxycarbonylamino, aminocarbonyl, mono- or di-(C₁-C₄)-alkylaminocarbonyl, amidosulfone, mono- or di-(C₁-C₄)-alkylamidosulfone,
R⁹ is hydrogen,
or a salt, hydrate, hydrate of the salt or solvate thereof.

4. A process for preparing the compounds of the formula (I) as defined in claim 1, characterized in that either
[A] a compound of the formula (V) 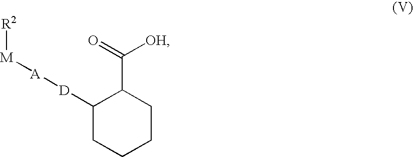
in which A, D, M and R² have the meanings stated in claim 1,
is reacted with a compound of the formula (VI) 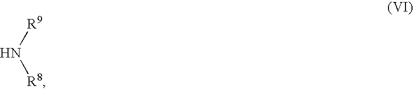
in which R⁸ and R⁹ have the meanings stated in claim 1,
in a solvent, where appropriate in the presence of a base and/or of a condensing agent,
or
[B] a compound of the formula (VII) 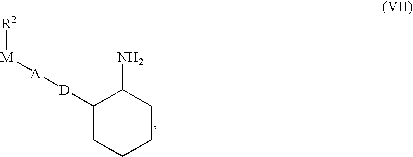
in which A, D, M and R² have the meanings stated in claim 1,
is reacted successively in any sequence with a compound of the formula (VIIIa) or (VIIIb)
R¹⁰—W (VIIIa)
R¹¹—W′ (VIIIb),
in which R¹⁰ and R¹¹ have the meanings stated in claim 1, and W and W′ are suitable leaving groups,
in a solvent, where appropriate in the presence of a base,
or
[C] a compound of the formula (Ia) 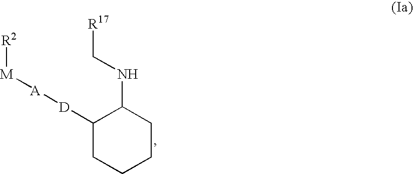
in which A, D, M, R² and R¹⁷ have the meanings stated in claim 1, is reacted in a solvent, where appropriate in the presence of a base, with a compound of the formula (VIIIb)
R¹¹—W′ (VIIIb),
in which R¹¹ and W′ have the meanings stated in claim 1,
or
[D] a compound of the formula (XII) 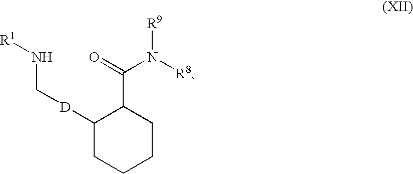
in which D, R¹, R⁸ and R⁹ have the meanings stated in claim 1,
is reacted either with a phosgene equivalent in a solvent and subsequently in a solvent, where appropriate in the presence of a base, with a compound of the formula (XIII)
HNR⁵R⁶ (XIII),
in which R⁵ and R⁶ have the meanings stated in claim 1,
or
in a solvent, where appropriate in the presence of a base, with a compound of the formula (XIV) 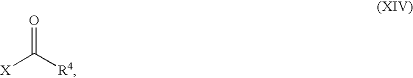
in which
X is a leaving group, and
R⁴ has the meaning stated in claim 1 with the exception of NR⁵R⁶,
or
in a solvent, where appropriate in the presence of a base, with a compound of the formula (XV) 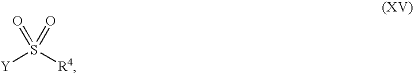
in which
Y is a leaving group, and
R⁴ has the abovementioned meaning,
or
[E] a compound of the formula (XX) 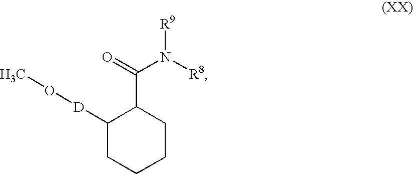
in which D, R⁸ and R⁹ have the meanings stated in claim 1,
is, after elimination of the methyl protective group, reacted with a compound of the formula (IIIb)
R²—V′ (IIIb)
in which R² and V′ have the meanings stated in claim 1.

5. (Canceled)

6. A pharmaceutical composition comprising at least one compound of the formula (I) as defined in claim 1, and at least one further pharmaceutically active substance.

7. A pharmaceutical composition comprising at least one compound of the formula (I) as defined in claim 1, and at least one further excipient.

8. A method for the prevention and/or treatment of cardiovascular diseases, comprising administering an effective amount of a compound of the formula (I) as defined in claim 1.

9. A method for the prevention and/or treatment of diseases of the urogenital tract, comprising administering an effective amount of a compound of the formula (I) as defined in claim 1.

10. A method for the prevention and/or treatment of cerebrovascular diseases, comprising administering an effective amount of a compound of the formula (I) as defined in claim 1.

11. The process of claim 4 wherein in the formulae (VIIIa) and (VIIIb), the groups W and W are halogen.

12. The process of claim 4 wherein in the formulae (VIIIa) and (VIIIb), the groups W and W are chlorine and bromine.

13. The process of claim 4 wherein the phosgene equivalent with which compound (XII) reacts is trichloromethyl chloroformate.

14. The process of claim 4 wherein in compound (XIV), X is the corresponding anhydride or a halogen.

15. The process of claim 4 wherein in compound (XIV), X is chlorine.

16. The process of claim 4 wherein in compound (XV), Y is a halogen.

17. The process of claim 4 wherein in compound (XV), Y is chlorine.
